(19) United States
(12) Patent Application Publication
(10) Pub. No.: US 2015/0139979 A1
(43) Pub. Date: May 21, 2015

(54) METHODS OF TREATING A DISEASE OR DISORDER ASSOCIATED WITH BRUTON'S TYROSINE KINASE

(71) Applicant: Celgene Avilomics Research, Inc., Bedford, MA (US)

(72) Inventors: Tom Daniel, LaJolla, CA (US); Kenichi Takeshita, New York, NY (US); Kenneth Foon, Summit, NJ (US); Jay Mei, North Wales, PA (US)

(73) Assignee: Celgene Avilomics Research, Inc., Bedford, MA (US)

(21) Appl. No.: 14/084,201

(22) Filed: Nov. 19, 2013

(51) Int. Cl. A61K 31/505; A61K 39/395; A61K 31/185; A61K 31/454

(52) U.S. Cl. CPC A61K 31/505; A61K 31/454; A61K 39/3955; A61K 31/185
USPC 424/133.1; 514/275

(57) ABSTRACT

The present invention provides methods of treating, stabilizing or lessening the severity or progression of a disease or disorder associated with BTK.

### FIELD OF THE INVENTION

The present invention provides methods of treating, stabilizing or lessening the severity or progression of a disease or disorder associated with Bruton's Tyrosine Kinase (“BTK”).

### BACKGROUND OF THE INVENTION

The search for new therapeutic agents has been greatly aided in recent years by a better understanding of the structure of enzymes and other biomolecules associated with diseases. One important class of enzymes that has been the subject of extensive study is protein kinases.

Protein kinases constitute a large family of structurally related enzymes that are responsible for the control of a variety of signal transduction processes within the cell. Protein kinases are thought to have evolved from a common ancestral gene due to the conservation of their structure and catalytic function. Almost all kinases contain a similar 250-300 amino acid catalytic domain. The kinases may be categorized into families by the substrates they phosphorylate (e.g., protein-tyrosine, protein-serine/threonine, lipids, etc.).

In general, protein kinases mediate intracellular signaling by effecting a phosphoryl transfer from a nucleoside triphosphate to a protein acceptor that is involved in a signaling pathway. These phosphorylation events act as molecular on/off switches that can modulate or regulate the target protein biological function. These phosphorylation events are ultimately triggered in response to a variety of extracellular and other stimuli. Examples of such stimuli include environmental and chemical stress signals (e.g., osmotic shock, heat shock, ultraviolet radiation, bacterial endotoxin, and H₂O₂), cytokines (e.g., interleukin-1 (IL-1) and tumor necrosis factor α (TNF-α)), and growth factors (e.g., granulocyte macrophage-colony-stimulating factor (GM-CSF), and fibroblast growth factor (FGF)). An extracellular stimulus may affect one or more cellular responses related to cell growth, migration, differentiation, secretion of hormones, activation of transcription factors, glucose metabolism, control of protein synthesis, and regulation of the cell cycle.

Many diseases are associated with abnormal cellular responses triggered by protein kinase-mediated events as described above. These diseases include, but are not limited to, autoimmune diseases, inflammatory diseases, bone diseases, metabolic diseases, neurological and neurodegenerative diseases, cancer, cardiovascular diseases, allergies and asthma, Alzheimer's disease, and hormone-related diseases. Accordingly, there remains a need to find protein kinase inhibitors useful as therapeutic agents.

### SUMMARY OF THE INVENTION

Non-Hodgkin lymphoma (NHL) is the fifth most common cancer in the West for both men and women. NHL is a heterogeneous group of lymphoproliferative malignancies with differing patterns of behavior and responses to treatment. The prognosis depends on the histological type, stage, and treatment. Most (approximately 80-90%) of the NHLs are of B-cell origin. B-cell non-Hodgkin lymphoma can be divided into 2 general prognostic groups: the indolent lymphomas and the aggressive lymphomas. Indolent lymphomas have a relatively good prognosis, with median survival time as long as 10 years, but they are not usually curable in advanced stages. Aggressive NHL constitutes about half of all cases of NHL in North America and Western Europe. The aggressive type of NHL has a shorter natural history; approximately 50-60% of these subjects can be cured with combination chemoimmunotherapy regimens.

Diffuse large cell lymphoma (DLBCL) and mantle cell lymphoma (MCL) are two histological subtypes under the category of aggressive B-cell non-Hodgkin lymphoma. Both histological types have seen advances in treatments in the last ten years. However, a substantial proportion of patients are not cured, and for such patients who relapse, better treatments are needed.

Bruton's tyrosine kinase (BTK) is a non-receptor tyrosine kinase with restricted cellular expression largely limited to B-lymphocytes, monocytes, and mast cells or basophils. BTK is a critical component of the B-cell receptor (BCR) signaling network and is crucial for B-cell development. Investigation has revealed that some B-cell malignancies, including B-cell non-Hodgkin lymphomas, depend on BCR signaling, suggesting that interruption of such signaling could be a promising therapeutic opportunity. Recently, clinical anti-tumor responses in various B-cell non-Hodgkin lymphoma (B-NHL) and CLL/SLL have been reported with agents that inhibit spleen tyrosine kinase (SYK) and BTK, both components of the BCR signaling pathway.

Recent preclinical research has shown that BTK is an important signaling protein in the pathway for lymphomagenesis, especially in certain types of DLBCL. Recent clinical research has further shown that both lenalidomide and certain BTK inhibitors exhibit activity in DLBCL and MCL, and lenalidomide has recently been approved in the United States for treatment of relapsed MCL.

United States published patent application number US 2010/0029610, published Feb. 4, 2010 (“the '610 publication,” the entirety of which is hereby incorporated herein by reference), describes certain 2,4-disubstituted pyrimidine compounds which covalently and irreversibly inhibit activity of one or more protein kinases, including BTK, a member of TEC-kinases. Such compounds include N-(3-(5-fluoro-2-(4-(2-methoxyethoxyl)phenylamino)pyrimidin-4-ylamino)phenyl)acrylamide, hereinafter referred to as Compound 1, which is designated as compound number I-182 in the '610 publication. The synthesis of Compound 1 is described in detail at Example 20 of the '610 publication Compound 1 is active in a variety of assays and therapeutic models demonstrating covalent, irreversible inhibition of BTK (in enzymatic and cellular assays). Notably, Compound 1 is a potent, selective, orally available, small molecule which was found to inhibit B-cell proliferation and activation. Compound 1 is therefore useful for treating one or more disorders associated with activity of BTK.

Accordingly, among other things, the present invention provides methods of treating, stabilizing or lessening the severity or progression of one or more diseases and conditions associated with BTK. In some aspects, the present invention provides methods of treating, stabilizing or lessening the severity or progression of one or more diseases and conditions associated with BTK comprising administering to a patient in need thereof a pharmaceutically acceptable composition comprising N-(3-(5-fluoro-2-(4-(2-methoxyethoxyl)phenylamino)pyrimidin-4-ylamino)phenyl)acrylamide (1):

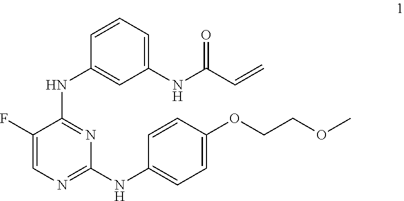 [SMILES: C=CC(=O)Nc1cccc(Nc2nc(Nc3ccc(OCCOC)cc3)ncc2F)c1]

or a pharmaceutically acceptable salt thereof, in combination with lenalidomide.

In some embodiments, the present invention provides a method of treating, stabilizing or lessening the severity or progression of a B-cell non-Hodgkin lymphoma, the method comprising administering to a patient in need thereof Compound 1 in combination with lenalidomide.

In some embodiments, the present invention provides a method of treating, stabilizing or lessening the severity or progression of a B-cell non-Hodgkin lymphoma, the method comprising administering to a patient in need thereof a composition comprising Compound 1 in combination with a composition comprising lenalidomide.

In some embodiments, the B-cell non-Hodgkin lymphoma is a B-cell non-Hodgkin lymphoma excluding SLL and related disorders such as CLL.

In some embodiments, provided methods comprise administering to a patient in need thereof Compound 1 in combination with lenalidomide, wherein Compound 1 is administered once a day (QD). In some embodiments, provided methods comprise administering to a patient in need thereof Compound 1 in combination with lenalidomide, wherein Compound 1 is administered twice a day (BID). In some such embodiments, lenalidomide is administered once a day (QD). Accordingly, in some embodiments, provided methods comprise administering to a patient in need thereof Compound 1 in combination with lenalidomide, wherein Compound 1 is administered twice a day (BID) and lenalidomide is administered once a day (QD).

In some embodiments, provided methods comprise administering to a patient in need thereof a composition comprising Compound 1 and lenalidomide.

In some embodiments, the present invention provides a method of treating, stabilizing or lessening the severity or progression of one or more diseases and conditions associated with BTK comprising administering to a patient in need thereof each of Compound 1, lenalidomide and an anti-CD20 antibody (e.g., rituximab). In some such embodiments, the disease or condition associated with BTK is a B-cell non-Hodgkin lymphoma.

In some embodiments, the B-cell non-Hodgkin lymphoma is indolent or aggressive. In some embodiments, the aggressive B-cell non-Hodgkin lymphoma is selected from diffuse large cell lymphoma (DLBCL) and mantle cell lymphoma (MCL).

In some embodiments, provided therapies comprise orally administering to a patient Compound 1 in combination with lenalidomide. In some embodiments, each of Compound 1 and lenalidomide is administered in the form of a pharmaceutical formulation. In some such embodiments, such formulations are capsule formulations.

In some embodiments, the present invention also provides dosing regimens and protocols for administering to patients in need thereof Compound 1 in combination with lenalidomide. Such methods, dosing regimens and protocols for the administration of said combination are described in further detail, below.

### BRIEF DESCRIPTION OF THE DRAWINGS

FIG. 1 presents a particular dose response curve for Compound 1 besylate in combination with lenalidomide (3000 nM) in the OCI-LY-10 cell line. =Compound 1 besylate; □=lenalidomide; Δ=calculated or expected activity of combination; ⋄=observed activity of the combination. Compound 1 besylate and lenalidomide appear to show an additive effect in the OCI-LY-10 cell line.

FIG. 2 presents a “volcano” plot of Compound 1 besylate (0.2 nM-1500 nM) in combination with lenalidomide (300 nM, 1000 nM and 3000 nM) in the OCI-LY-10 cell line. Compound 1 besylate and lenalidomide appear to show an additive effect in the OCI-LY-10 cell line.

FIG. 3 presents a particular dose response curve for Compound 1 besylate in combination with lenalidomide (3333 nM) in the WSU-DLCL2 cell line. =Compound 1 besylate; □=lenalidomide; Δ=calculated or expected activity of combination; ⋄=observed activity of the combination. Compound 1 besylate and lenalidomide appear to show an additive effect in the WSU-DLCL2 cell line.

FIG. 4 presents a “volcano” plot of Compound 1 besylate (0.5 nM-3333 nM) in combination with lenalidomide (123 nM, 3333 nM and 10000 nM) in the WSU-DLCL2 cell line. Compound 1 besylate and lenalidomide appear to show an additive effect in the WSU-DLCL2 cell line.

FIG. 5 presents a particular dose response curve for Compound 1 besylate in combination with lenalidomide (3333 nM) in the Riva cell line. =Compound 1 besylate; □=lenalidomide; Δ=calculated or expected activity of combination; ⋄=observed activity of the combination. Apparent synergistic effect is indicated by the arrow.

FIG. 6 presents a “volcano” plot of Compound 1 besylate (0.2 nM-1000 nM) in combination with lenalidomide (41 nM, 1111 nM and 3333 nM) in the Riva cell line. Apparent synergistic effect is indicated by the arrow.

FIG. 7 presents a particular dose response curve for Compound 1 besylate in combination with lenalidomide (333 nM) in the Riva cell line. =Compound 1 besylate; □=lenalidomide; Δ=calculated or expected activity of combination; ⋄=observed activity of the combination. Apparent synergy is indicated by the arrow.

FIG. 8 presents a “volcano” plot of Compound 1 besylate (0.2 nM-1000 nM) in combination with lenalidomide (41 nM, 123 nM and 370 nM) in the Riva cell line. Apparent synergy is indicated by the arrow.

FIG. 9 presents a particular dose response curve for Compound 1 besylate in combination with lenalidomide (333 nM) in the Riva cell line. =Compound 1 besylate; □=lenalidomide; Δ=calculated or expected activity of combination; ⋄=observed activity of the combination.

FIG. 10 presents a “volcano” plot of Compound 1 besylate (0.2 nM-1000 nM) in combination with lenalidomide (41 nM, 123 nM and 370 nM) in the Riva cell line. Compound 1 besylate and lenalidomide appear to show an additive effect in the Riva cell line.

FIG. 11 presents a particular dose response curve for Compound 1 besylate in combination with lenalidomide (3000 nM) in the TMD-8 cell line. =Compound 1 besylate; □=lenalidomide; Δ=calculated or expected activity of combination; ⋄=observed activity of the combination. Apparent synergy is indicated by the arrow.

FIG. 12 presents a “volcano” plot of Compound 1 besylate (0.2 nM-1500 nM) in combination with lenalidomide (300 nM, 1000 nM and 3000 nM) in the TMD-8 cell line. Apparent synergy is indicated by the arrow.

FIG. 13 presents the Response Assessment for patients enrolled in cohort 1.

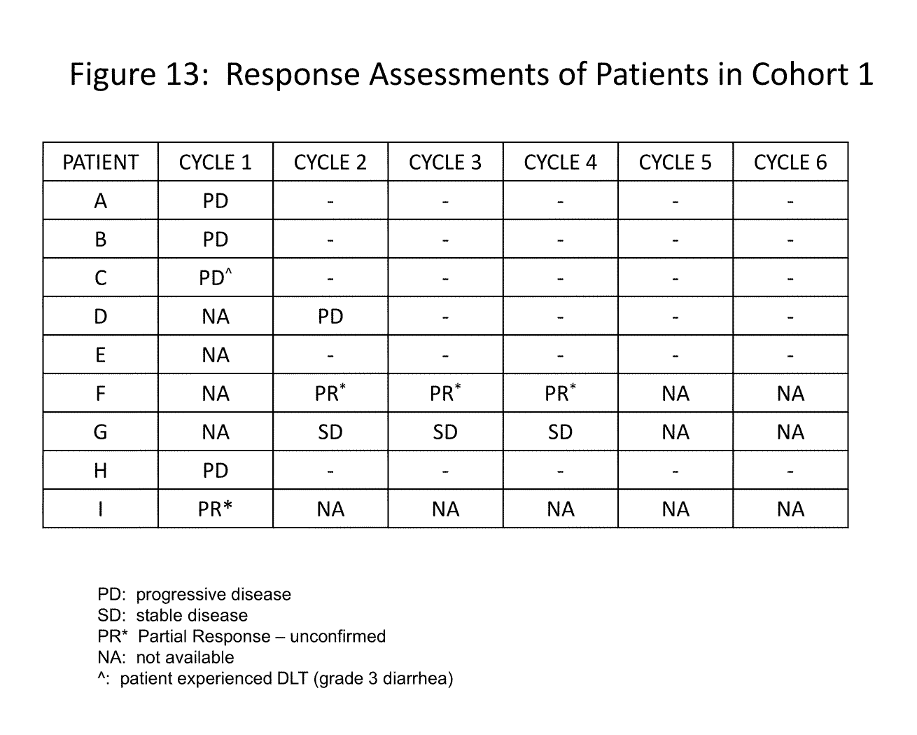

### DETAILED DESCRIPTION OF THE INVENTION

### Definitions

As used herein, the terms “combination”, “in combination with” or “combination therapy” refer to those situations in which two or more different pharmaceutical agents are administered in overlapping regimens so that the subject is simultaneously exposed to both agents. In some embodiments, such combinations refer to simultaneously administering to a subject separate dosage forms of Compound 1 and lenalidomide. In some embodiments, such combinations refer to contemporaneously administering to a subject separate dosage forms of Compound 1 and lenalidomide, wherein Compound 1 is administered before, during or after administration of lenalidomide. In some embodiments, simultaneous or contemporaneous exposure of Compound 1 and lenalidomide is effected via different dosage regimens appropriate for each therapeutic agent. For example, in some embodiments, Compound 1 is administered twice daily, or BID, whereas lenalidomide is administered once daily, or QD. Alternatively and/or additionally, Compound 1 may be administered once or twice daily for one or more 28-day cycles, whereas lenalidomide may be administered once daily for days 1 through 21 of one or more 28-day cycles. In some embodiments, Compound 1 is administered twice daily on days 1 through 28 of one or more 28-day cycles and lenalidomide is administered once daily on days 2 through 22 of one or more 28-day cycles. In some embodiments, Compound 1 is administered twice daily on days 1 through 28 of one or more 28-day cycles and lenalidomide is administered once daily on days 1 through 28 of one or more 28-day cycles.

The term “percent inhibition” as used herein refers to the percent decrease of target activity in the presence of a test compound (e.g., an irreversible BTK inhibitor) relative to control target activity. It will be appreciated that percent inhibition of a target (e.g., a kinase) can be determined in numerous ways, one of which is described in Example 3, infra. In some embodiments, percent inhibition is expressed as % inhibition (e.g., 50% inhibition). In some embodiments, the percent inhibition of a kinase is an average percent inhibition.

As used herein, the term “comparable”, refers to two or more agents, entities, situations, sets of conditions, etc. that may not be identical to one another but that are sufficiently similar to permit comparison therebetween so that conclusions may reasonably be drawn based on differences or similarities observed. Those of ordinary skill in the art will understand, in context, what degree of identity is required in any given circumstance for two or more such agents, entities, situations, sets of conditions, etc. to be considered comparable. As used herein, the terms “comparable percent inhibition” or “comparable average percent inhibition” refer to a percent inhibition or an average percent inhibition, respectively, of a kinase that is within 10% of that observed or determined for a reference kinase inhibitor. For example, if a reference kinase inhibitor has 50% inhibition of a kinase relative to a control, another inhibitor will be considered to show comparable inhibition if it has about 40% to about 60% inhibition of the same kinase relative to the control. In some embodiments, an irreversible BTK inhibitor has comparable percent inhibition to a reference kinase inhibitor wherein the percent inhibition of the irreversible BTK inhibitor is within 9%, 8%, 7%, 6%, 5%, 4%, 3%, 2% or 1% inhibition of that observed or determined for a reference kinase inhibitor.

As used herein, a “disease or disorder associated with BTK” or a “BTK-mediated disorder” means any disease or other deleterious condition in which BTK, or a mutant thereof, is known or suspected to play a role. Accordingly, another embodiment of the present invention relates to preventing, treating, stabilizing or lessening the severity or progression of one or more diseases in which BTK, or a mutant thereof, is known or suspected to play a role. Specifically, the present invention relates to a method of treating or lessening the severity of a proliferative disorder, wherein said method comprises administering to a patient in need thereof Compound 1 in combination with lenalidomide.

As used herein, the term “irreversible” or “irreversible inhibitor” refers to an inhibitor (i.e. a compound) that is able to be covalently bonded to a target protein kinase in a substantially non-reversible manner. That is, whereas a reversible inhibitor is able to bind to (but is generally unable to form a covalent bond to) the target protein kinase, and therefore can become dissociated from the target protein kinase, an irreversible inhibitor will remain substantially bound to the target protein kinase once covalent bond formation has occurred. Irreversible inhibitors usually display time dependency, whereby the degree of inhibition increases with the time with which the inhibitor is in contact with the enzyme. Methods for identifying if a compound is acting as an irreversible inhibitor are known to one of ordinary skill in the art. Such methods include, but are not limited to, enzyme kinetic analysis of the inhibition profile of the compound with the protein kinase target, the use of mass spectrometry of the protein drug target modified in the presence of the inhibitor compound, discontinuous exposure, also known as “washout,” experiments, and the use of labeling, such as radiolabelled inhibitor, to show covalent modification of the enzyme, as well as other methods known to one of skill in the art.

The term “refractory B-cell non-Hodgkin lymphoma” as used herein is defined as B-cell non-Hodgkin lymphoma which was treated with a rituximab-containing regimen (i) without achieving at least a partial response to therapy or (ii) which progressed within 6 months of treatment.

The term “relapsed B-cell non-Hodgkin lymphoma” as used herein is defined as B-cell non-Hodgkin lymphoma which progressed after ≧6 months post-treatment with a rituximab-containing regimen after achieving partial response or complete response to therapy.

As used herein, the term “antibody”, or grammatical variations thereof (i.e., antibodies), refers to polypeptide(s) capable of binding to an epitope. In some embodiments, an antibody is a full-length antibody. In some embodiments, an antibody is less than full length (i.e., an antibody fragment) but includes at least one binding site. In some such embodiments, the binding site comprises at least one, and preferably at least two sequences with structure of antibody variable regions. In some embodiments, the term “antibody” encompasses any protein having a binding domain which is homologous or largely homologous to an immunoglobulin-binding domain. In particular embodiments, the term “antibody” encompasses polypeptides having a binding domain that shows at least 99% identity with an immunoglobulin-binding domain. In some embodiments, the antibody is any protein having a binding domain that shows at least 70%, at least 80%, at least 85%, at least 90% or at least 95% identity with an immunoglobulin-binding domain. Antibody polypeptides in accordance with the present invention may be prepared by any available means, including, for example, isolation from a natural source or antibody library, recombinant production in or with a host system, chemical synthesis, etc., or combinations thereof. In some embodiments, an antibody is monoclonal or polyclonal. In some embodiments, an antibody may be a member of any immunoglobulin class, including any of the human classes IgG, IgM, IgA, IgD and IgE. In certain embodiments, an antibody is a member of the IgG immunoglobulin class. In some embodiments, the term “antibody” refers to any derivative of an antibody that possesses the ability to bind to an epitope of interest. In some embodiments, an antibody fragment comprises multiple chains that are linked together, for example, by disulfide linkages. In some embodiments, an antibody is a human antibody. In some embodiments, an antibody is a humanized antibody. In some embodiments, humanized antibodies include chimeric immunoglobulins, immunoglobulin chains or antibody fragments (Fv, Fab, Fab′, F(ab′)₂ or other antigen binding subsequences of antibodies) that contain minimal sequence derived from non-human immunoglobulin. In some embodiments, humanized antibodies are human immunoglobulin (recipient antibody) in which residues from a complementary-determining region (CDR) of the recipient are replaced by residues from a CDR of a non-human species (donor antibody) such as mouse, rat or rabbit having the desired specificity, affinity and capacity. In particular embodiments, antibodies for use in the present invention bind to particular epitopes of CD20. In some embodiments, epitopes of CD20 to which anti-CD20 antibodies bind include, for example, ¹⁷⁰ANPS¹⁷³ (Binder et al., Blood 2006, 108(6): 1975-1978), FMC7 (Deans et al., Blood 2008, 111(4): 2492), Rp5-L and Rp15-C (mimotopes of CD20) (Perosa et al., J. Immunol. 2009, 182:416-423), ¹⁸²YCYSI¹⁸⁵ (Binder et al., Blood 2006, 108(6): 1975-1978) and WEWTI (a mimic of ¹⁸²YCYSI¹⁸⁵) (Binder et al., Blood 2006, 108(6): 1975-1978). In some embodiments, an anti-CD20 antibody has a binding affinity (K_{d}) for an epitope of CD20 of less than 12 nM, less than 11 nM, less than 10 nM, less than 9 nM, less than 8 nM, less than 7 nM, less than 6 nM, less than 5 nM, less than 4 nM, less than 3 nM, less than 2 nM or less than 1 nM.

As used herein, the term “biosimilar” (for example, of an approved reference product/biological drug, such as a protein therapeutic, antibody, etc.) refers to a biologic product that is similar to the reference product based upon data derived from (a) analytical studies that demonstrate that the biological product is highly similar to the reference product notwithstanding minor differences in clinically inactive components; (b) animal studies (including the assessment of toxicity); and/or (c) a clinical study or studies (including the assessment of immunogenicity and pharmacokinetics or pharmacodynamics) that are sufficient to demonstrate safety, purity, and potency in one or more appropriate conditions of use for which the reference product is approved and intended to be used and for which approval is sought (e.g., that there are no clinically meaningful differences between the biological product and the reference product in terms of the safety, purity, and potency of the product).

In some embodiments, the biosimilar biological product and reference product utilizes the same mechanism or mechanisms of action for the condition or conditions of use prescribed, recommended, or suggested in the proposed labeling, but only to the extent the mechanism or mechanisms of action are known for the reference product. In some embodiments, the condition or conditions of use prescribed, recommended, or suggested in the labeling proposed for the biological product have been previously approved for the reference product. In some embodiments, the route of administration, the dosage form, and/or the strength of the biological product are the same as those of the reference product. In some embodiments, the facility in which the biological product is manufactured, processed, packed, or held meets standards designed to assure that the biological product continues to be safe, pure, and potent. The reference product may be approved in at least one of the U.S., Europe, or Japan. A biosimilar can be for example, a presently known antibody having the same primary amino acid sequence as a marketed antibody, but may be made in different cell types or by different production, purification or formulation methods.

The term “subject”, as used herein, means a mammal and includes human and animal subjects, such as domestic animals (e.g., horses, dogs, cats, etc.).

As used herein, a “therapeutically effective amount” means an amount of a substance (e.g., a therapeutic agent, composition, and/or formulation) that elicits a desired biological response. In some embodiments, a therapeutically effective amount of a substance is an amount that is sufficient, when administered as part of a dosing regimen to a subject suffering from or susceptible to a disease, disorder, and/or condition, to treat, diagnose, prevent, and/or delay the onset of the disease, disorder, and/or condition. As will be appreciated by those of ordinary skill in this art, the effective amount of a substance may vary depending on such factors as the desired biological endpoint, the substance to be delivered, the target cell or tissue, etc. For example, the effective amount of compound in a formulation to treat a disease, disorder, and/or condition is the amount that alleviates, ameliorates, relieves, inhibits, prevents, delays onset of, reduces severity of and/or reduces incidence of one or more symptoms or features of the disease, disorder, and/or condition. In some embodiments, a “therapeutically effective amount” is at least a minimal amount of a compound, or composition containing a compound, which is sufficient for treating one or more symptoms of a disorder or condition associated with Bruton's tyrosine kinase.

The terms “treat” or “treating,” as used herein, refers to partially or completely alleviating, inhibiting, delaying onset of, preventing, ameliorating and/or relieving a disorder or condition, or one or more symptoms of the disorder or condition. As used herein, the terms “treatment,” “treat,” and “treating” refer to partially or completely alleviating, inhibiting, delaying onset of, preventing, ameliorating and/or relieving a disorder or condition, or one or more symptoms of the disorder or condition, as described herein. In some embodiments, treatment may be administered after one or more symptoms have developed. In some embodiments, the term “treating” includes preventing or halting the progression of a disease or disorder. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence. Thus, in some embodiments, the term “treating” includes preventing relapse or recurrence of a disease or disorder.

The expression “unit dosage form” as used herein refers to a physically discrete unit of therapeutic formulation appropriate for the subject to be treated. It will be understood, however, that the total daily usage of the compositions of the present invention will be decided by the attending physician within the scope of sound medical judgment. The specific effective dose level for any particular subject or organism will depend upon a variety of factors including the disorder being treated and the severity of the disorder; activity of specific active agent employed; specific composition employed; age, body weight, general health, sex and diet of the subject; time of administration, and rate of excretion of the specific active agent employed; duration of the treatment; drugs and/or additional therapies used in combination or coincidental with specific compound(s) employed, and like factors well known in the medical arts.

### General Methods of Treating a BTK-Mediated Disease or Disorder

In some embodiments, the present invention provides a method of treating, stabilizing or lessening the severity or progression of one or more diseases and conditions associated with BTK comprising administering to a patient in need thereof an irreversible BTK inhibitor and lenalidomide.

It is understood that although the methods described herein refer to formulations, doses and dosing regimens/schedules of Compound 1 and salts thereof, such formulatiosn, doses and/or dosing regimens/schedules are equally applicable to any irreversible BTK inhibitor, such as those described below. Accordingly, in some embodiments, a dose or dosing regimen of an irreversible BTK inhibitor is selected from any of the doses or dosing regimens for Compound 1 as described herein. In some embodiments, provided methods comprise administering an irreversible BTK inhibitor in an amount selected from any of the doses for Compound 1 as described herein. In some such embodiments, a dose of an irreversible BTK inhibitor is administered according to a dosing schedule selected from any of the dosing schedules described herein for Compound 1. In some embodiments, a composition comprising an irreversible BTK inhibitor is any of the formulations as described herein.

In some embodiments, the present invention provides a method of treating, stabilizing or lessening the severity or progression of one or more diseases and conditions associated with BTK comprising administering to a patient in need thereof an irreversible BTK inhibitor, lenalidomide and an anti-CD20 antibody (e.g., rituximab).

In some embodiments, the irreversible BTK inhibitor covalently binds to Cys 481 of BTK.

In some embodiments, an irreversible BTK inhibitor has activity against one or more kinases selected from the kinases recited in Table 5, infra.

In some embodiments, an irreversible BTK inhibitor has a percent inhibition comparable to that of a reference kinase inhibitor with respect to a kinase selected from Table 5, or combinations thereof. In some such embodiments, the reference kinase inhibitor is Compound 2:

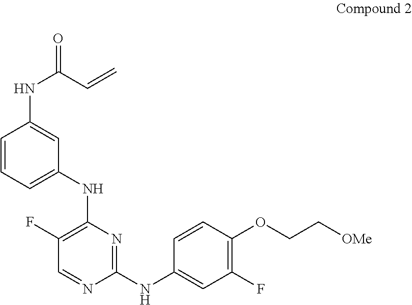 [SMILES: C=CC(=O)Nc1cccc(Nc2nc(Nc3ccc(OCCOC)c(F)c3)ncc2F)c1]

In some embodiments, the percent inhibition of the reference kinase inhibitor is that shown for Compound 2 in Example 3.

In some embodiments, an irreversible BTK inhibitor has a percent inhibition comparable to that of Compound 2 with respect to one or more kinases selected from Table 5, or combinations thereof, in that the irreversible kinase inhibitor has a percent inhibition within approximately 10% of that observed for Compound 2. In some embodiments, an irreversible BTK inhibitor has a percent inhibition comparable to that of Compound 2 with respect to one or more kinases selected from Table 5, or combinations thereof, in that the irreversible kinase inhibitor has a percent inhibition that is within about 9%, or about 8%, or about 7%, or about 6%, or about 5%, or about 4%, or about 3%, or about 2% or about 1% inhibition of that observed for Compound 2.

In some embodiments, an irreversible BTK inhibitor has a percent inhibition that is greater than that observed for Compound 2 with respect to one or more kinases selected from Table 5. In some embodiments, an irreversible BTK inhibitor has a percent inhibition that is less than that observed for Compound 2 with respect to one or more kinases selected from Table 5.

In some embodiments, the irreversible BTK inhibitor has a percent inhibition of one or more additional kinases, wherein the percent inhibition of the kinase or kinases is at least about 50%, at least about 55%, at least about 60%, at least about 65%, at least about 70%, at least about 75%, at least about 80%, at least about 85%, at least about 90% or at least about 95%.

In some embodiments, the irreversible BTK inhibitor has a percent inhibition comparable to that of a reference kinase inhibitor with respect to one or more kinases selected from the group consisting of TXK, BMX/ETK, FLT3, BLK, TEC, ERBB4/HER4, Aurora B, TRKC, RET, LOK/STK10, Aurora C, FLT4/VEGFR3, ROS/ROS1, ARK5/NUAK1, EGFR, DDR1, JAK3, LRRK2, ABL2/ARG, ITK, Aurora A, YESNES1, FGFR3, TNK1, BRK, FGFR2, PDGFRb, c-SRC, ACK1, FGFR1, STK16, ABL1, AXL, TYK2, ERBB2/HER2, FGR, CHK2, SIK1, MLK1/MAP3K9, MLK2/MAP3K10, MLK3/MAP3K11, PKCb2 and CLK2, or combinations thereof. In some embodiments, the reference kinase inhibitor is Compound 2. In some embodiments, the percent inhibition of the reference kinase inhibitor is that shown for Compound 2 in Example 3.

In some embodiments, the irreversible BTK inhibitor has a percent inhibition comparable to that of a reference kinase inhibitor with respect to the group of kinases consisting of TXK, BMX/ETK, FLT3, BLK, TEC, ERBB4/HER4, Aurora B, TRKC, RET, LOK/STK10, Aurora C, FLT4/VEGFR3, ROS/ROS1, ARK5/NUAK1, EGFR, DDR1, JAK3, LRRK2, ABL2/ARG, ITK, Aurora A, YES/YES1, FGFR3, TNK1, BRK, FGFR2, PDGFRb, c-SRC, ACK1, FGFR1, STK16, ABL1, AXL, TYK2, ERBB2/HER2, FGR, CHK2, SIK1, MLK1/MAP3K9, MLK2/MAP3K10, MLK3/MAP3K11, PKCb2 and CLK2, or combinations thereof. In some embodiments, the reference kinase inhibitor is Compound 2. In some embodiments, the percent inhibition of the reference kinase inhibitor is that shown for Compound 2 in Example 3.

In some embodiments, the irreversible BTK inhibitor inhibits a kinase selected from the group consisting of TXK, BMX/ETK, FLT3, BLK, TEC, ERBB4/HER4, Aurora B, TRKC, RET, LOK/STK10, Aurora C, FLT4/VEGFR3, ROS/ROS1, ARK5/NUAK1, EGFR, DDR1, JAK3, LRRK2, ABL2/ARG, ITK, Aurora A, YES/YES1, FGFR3, TNK1, BRK, FGFR2, PDGFRb, c-SRC, ACK1, FGFR1, STK16, ABL1, AXL, TYK2, ERBB2/HER2, FGR, CHK2, SIK1, MLK1/MAP3K9, MLK2/MAP3K10, MLK3/MAP3K11, PKCb2 and CLK2, or combinations thereof, wherein the inhibition of the kinase or kinases is at least the percent inhibition observed for a reference kinase inhibitor. In some embodiments, the reference kinase inhibitor is Compound 2. In some embodiments, the percent inhibition of the reference kinase inhibitor is that shown for Compound 2 in Example 3.

In some embodiments, the irreversible BTK inhibitor has at least about 50%, at least about 55%, at least about 60%, at least 65%, at least about 70%, at least about 75%, at least about 80%, at least about 85%, at least about 90% or at least about 95% inhibition of a kinase selected from TXK, BMX/ETK, FLT3, BLK, TEC, ERBB4/HER4, Aurora B, TRKC, RET, LOK/STK10, Aurora C, FLT4/VEGFR3, ROS/ROS1, ARK5/NUAK1, EGFR, DDR1, JAK3, LRRK2, ABL2/ARG, ITK, Aurora A, YES/YES1, FGFR3, TNK1, BRK, FGFR2, PDGFRb, c-SRC, ACK1, FGFR1, STK16, ABL1, AXL, TYK2, ERBB2/HER2, FGR, CHK2, SIK1, MLK1/MAP3K9, MLK2/MAP3K10, MLK3/MAP3K11, PKCb2 and CLK2, or combinations thereof.

In some embodiments, the irreversible BTK inhibitor has a percent inhibition comparable to that of a reference kinase inhibitor with respect to one or more kinases selected from the group consisting of Aurora A, Aurora B, Aurora C, TRKC, ROS/ROS1, ARK5/NUAK1, LRRK2, ABL2/ARG, TNK1, STK16, ABL1, AXL, TYK2, CHK2, SIK1, MLK1/MAP3K9, MLK2/MAP3K10, MLK3/MAP3K11, PKCb2 and CLK2, or combinations thereof. In some embodiments, the reference kinase inhibitor is Compound 2. In some embodiments, the percent inhibition of the reference kinase inhibitor is that shown for Compound 2 in Example 3.

In some embodiments, the irreversible BTK inhibitor has a percent inhibition comparable to that of a reference kinase inhibitor with respect to the group of kinases consisting of Aurora A, Aurora B, Aurora C, TRKC, ROS/ROS1, ARK5/NUAK1, LRRK2, ABL2/ARG, TNK1, STK16, ABL1, AXL, TYK2, CHK2, SIK1, MLK1/MAP3K9, MLK2/MAP3K10, MLK3/MAP3K11, PKCb2 and CLK2, or combinations thereof. In some embodiments, the reference kinase inhibitor is Compound 2. In some embodiments, the percent inhibition of the reference kinase inhibitor is that shown for Compound 2 in Example 3.

In some embodiments, the irreversible BTK inhibitor has a percent inhibition that is at least the percent inhibition observed for a reference kinase inhibitor with respect to one or more kinases selected from the group consisting of Aurora A, Aurora B, Aurora C, TRKC, ROS/ROS1, ARK5/NUAK1, LRRK2, ABL2/ARG, TNK1, STK16, ABL1, AXL, TYK2, CHK2, SIK1, MLK1/MAP3K9, MLK2/MAP3K10, MLK3/MAP3K11, PKCb2 and CLK2, or combinations thereof. In some embodiments, the reference kinase inhibitor is Compound 2. In some embodiments, the percent inhibition of the reference kinase inhibitor is that shown for Compound 2 in Example 3.

In some embodiments, the irreversible BTK inhibitor has at least about 50%, at least about 55%, at least about 60%, at least about 65%, at least about 70%, at least about 75%, at least about 80%, at least about 85%, at least about 90% or at least about 95% inhibition of a kinase selected from Aurora A, Aurora B, Aurora C, TRKC, ROS/ROS1, ARK5/NUAK1, LRRK2, ABL2/ARG, TNK1, STK16, ABL1, AXL, TYK2, CHK2, SIK1, MLK1/MAP3K9, MLK2/MAP3K10, MLK3/MAP3K11, PKCb2 and CLK2, or combinations thereof.

In some embodiments, the present invention provides a method of treating, stabilizing or lessening the severity or progression of CLL/SLL comprising administering to a patient in need thereof an irreversible BTK inhibitor in combination with lenalidomide, wherein the irreversible BTK inhibitor has not more than about 50% inhibition of a kinase selected from Aurora A, Aurora B, Aurora C, TRKC, ROS/ROS1, ARK5/NUAK1, LRRK2, ABL2/ARG, TNK1, STK16, ABL1, AXL, TYK2, CHK2, SIK1, MLK1/MAP3K9, MLK2/MAP3K10, MLK3/MAP3K11, PKCb2 and CLK2, or combinations thereof.

In some embodiments, the irreversible BTK inhibitor has at least about 50% inhibition of a kinase selected from Aurora A, Aurora B, Aurora C, TRKC, ROS/ROS1, ARK5/NUAK1, LRRK2, ABL2/ARG, TNK1, STK16, ABL1, AXL, TYK2, CHK2, SIK1, MLK1/MAP3K9, MLK2/MAP3K10, MLK3/MAP3K11 and PKCb2, or combinations thereof. In some embodiments, the irreversible BTK inhibitor has at least about 50% inhibition of the group of kinases consisting of Aurora A, Aurora B, Aurora C, TRKC, ROS/ROS1, ARK5/NUAK1, LRRK2, ABL2/ARG, TNK1, STK16, ABL1, AXL, TYK2, CHK2, SIK1, MLK1/MAP3K9, MLK2/MAP3K10, MLK3/MAP3K11, PKCb2 and CLK2.

In some embodiments, the irreversible BTK inhibitor has at least about 55% inhibition of a kinase selected from Aurora A, Aurora B, Aurora C, TRKC, ROS/ROS1, ARK5/NUAK1, LRRK2, ABL2/ARG, TNK1, STK16, ABL1, CHK2, MLK1/MAP3K9, MLK2/MAP3K10 and MLK3/MAP3K11, or combinations thereof. In some embodiments, the irreversible BTK inhibitor has at least about 55% inhibition of the group of kinases consisting of Aurora A, Aurora B, Aurora C, TRKC, ROS/ROS1, ARK5/NUAK1, LRRK2, ABL2/ARG, TNK1, STK16, ABL1, CHK2, MLK1/MAP3K9, MLK2/MAP3K10 and MLK3/MAP3K11.

In some embodiments, the irreversible BTK inhibitor has at least about 60% inhibition of a kinase selected from Aurora A, Aurora B, Aurora C, TRKC, ROS/ROS1, ARK5/NUAK1, LRRK2, ABL2/ARG, TNK1, STK16, CHK2, MLK1/MAP3K9 and MLK3/MAP3K11, or combinations thereof. In some embodiments, the irreversible BTK inhibitor has at least about 60% inhibition of the group of kinases consisting of Aurora A, Aurora B, Aurora C, TRKC, ROS/ROS1, ARK5/NUAK1, LRRK2, ABL2/ARG, TNK1, STK16, CHK2, MLK1/MAP3K9 and MLK3/MAP3K11.

In some embodiments, the irreversible BTK inhibitor has at least about 65% inhibition of a kinase selected from Aurora A, Aurora B, Aurora C, TRKC, ROS/ROS1, ARK5/NUAK1, LRRK2, TNK1, STK16, CHK2, MLK1/MAP3K9 and MLK3/MAP3K11, or combinations thereof. In some embodiments, the irreversible BTK inhibitor sh at least about 65% inhibition of the group of kinases consisting of Aurora A, Aurora B, Aurora C, TRKC, ROS/ROS1, ARK5/NUAK1, LRRK2, TNK1, STK16, CHK2, MLK1/MAP3K9 and MLK3/MAP3K11.

In some embodiments, the irreversible BTK inhibitor has at least about 70% inhibition of a kinase selected from Aurora A, Aurora B, Aurora C, ROS/ROS1, ARK5/NUAK1, TNK1, STK16, CHK2, MLK1/MAP3K9 and MLK3/MAP3K11, or combinations thereof. In some embodiments, the irreversible BTK inhibitor at least about 70% inhibition of the group of kinases consisting of Aurora A, Aurora B, Aurora C, ROS/ROS1, ARK5/NUAK1, TNK1, STK16, CHK2, MLK1/MAP3K9 and MLK3/MAP3K11.

In some embodiments, the irreversible BTK inhibitor has at least about 75% inhibition of a kinase selected from Aurora A, Aurora B, ROS/ROS1, ARK5/NUAK1, TNK1, STK16 and MLK1/MAP3K9, or combinations thereof. In some embodiments, the irreversible BTK inhibitor has at least about 75% inhibition of the group of kinases consisting of Aurora A, Aurora B, ROS/ROS1, ARK5/NUAK1, TNK1, STK16 and MLK1/MAP3K9.

In some embodiments, the irreversible BTK inhibitor has at least about 80% inhibition of a kinase selected from Aurora A, Aurora B, ROS/ROS1, ARK5/NUAK1, TNK1 and MLK1/MAP3K9, or combinations thereof. In some embodiments, the irreversible BTK inhibitor has at least about 80% inhibition of the group of kinases consisting of Aurora A, Aurora B, ROS/ROS1, ARK5/NUAK1, TNK1 and MLK1/MAP3K9.

In some embodiments, the irreversible BTK inhibitor has at least about 85% inhibition of a kinase selected from Aurora A, Aurora B, ROS/ROS1, ARK5/NUAK1 and MLK1/MAP3K9, or combinations thereof. In some embodiments, the irreversible BTK inhibitor has at least about 85% inhibition of the group of kinases consisting of Aurora A, Aurora B, ROS/ROS1, ARK5/NUAK1 and MLK1/MAP3K9.

In some embodiments, the irreversible BTK inhibitor has a percent inhibition comparable to that of a reference kinase inhibitor with respect to one or more kinases selected from the group consisting of TNK1, STK16, ABL1, AXL, TYK2, CHK2, MLK1/MAP3K9, MLK2/MAP3K10, MLK3/MAP3K11, SIK1, PKCb2 and CLK2, or combinations thereof. In some embodiments, the reference kinase inhibitor is Compound 2. In some embodiments, the percent inhibition of the reference kinase inhibitor is that shown for Compound 2 in Example 3.

In some embodiments, the irreversible BTK inhibitor has a percent inhibition comparable to that of a reference kinase inhibitor with respect to the group of kinases consisting of TNK1, STK16, ABL1, AXL, TYK2, CHK2, MLK1/MAP3K9, MLK2/MAP3K10, MLK3/MAP3K11, SIK1, PKCb2 and CLK2, or combinations thereof. In some embodiments, the reference kinase inhibitor is Compound 2. In some embodiments, the percent inhibition of the reference kinase inhibitor is that shown for Compound 2 in Example 3.

In some embodiments, the irreversible BTK inhibitor has a percent inhibition that is at least the percent inhibition observed for a reference kinase inhibitor with respect to one or more kinases selected from the group consisting of TNK1, STK16, ABL1, AXL, TYK2, CHK2, MLK1/MAP3K9, MLK2/MAP3K10, MLK3/MAP3K11, SIK1, PKCb2 and CLK2, or combinations thereof. In some embodiments, the reference kinase inhibitor is Compound 2. In some embodiments, the percent inhibition of the reference kinase inhibitor is that shown for Compound 2 in Example 3.

In some embodiments, the irreversible BTK inhibitor has at least about 50%, at least about 55%, at least about 60%, at least about 65%, at least about 70%, at least about 75%, at least about 80%, at least about 85%, at least about 90% or at least about 95% inhibition of a kinase selected from TNK1, STK16, ABL1, AXL, TYK2, CHK2, MLK1/MAP3K9, MLK2/MAP3K10, MLK3/MAP3K11, SIK1, PKCb2 and CLK2, or combinations thereof.

In some embodiments, the irreversible BTK inhibitor has at least about 50% inhibition of a kinase selected from TNK1, STK16, ABL1, AXL, TYK2, CHK2, MLK1/MAP3K9, MLK2/MAP3K10, MLK3/MAP3K11, SIK1, PKCb2 and CLK2, or combinations thereof. In some embodiments, the irreversible BTK inhibitor at least about 50% inhibition of the group of kinases consisting of TNK1, STK16, ABL1, AXL, TYK2, CHK2, MLK1/MAP3K9, MLK2/MAP3K10, MLK3/MAP3K11, SIK1, PKCb2 and CLK2.

In some embodiments, the irreversible BTK inhibitor has at least about 55% inhibition of a kinase selected from TNK1, STK16, ABL1, CHK2, MLK1/MAP3K9 and MLK3/MAP3K11, or combinations thereof. In some embodiments, the irreversible BTK inhibitor has at least about 55% inhibition of the group of kinases consisting of TNK1, STK16, ABL1, CHK2, MLK1/MAP3K9 and MLK3/MAP3K11, or combinations thereof.

In some embodiments, the irreversible BTK inhibitor has at least about 60%, at least about 65% or at least about 70% inhibition of a kinase selected from TNK1, STK16, CHK2, MLK1/MAP3K9 and MLK3/MAP3K11, or combinations thereof. In some embodiments, the irreversible BTK inhibitor has at least about 60%, at least about 65% or at least about 70% inhibition of the group of kinases consisting of CHK2, MLK1/MAP3K9 and MLK3/MAP3K11.

In some embodiments, the irreversible BTK inhibitor has at least about 75% inhibition of a kinase selected from TNK1, STK16 and MLK1/MAP3K9, or combinations thereof. In some embodiments, the irreversible BTK inhibitor has at least about 75% inhibition of the group of kinases consisting of TNK1, STK16 and MLK1/MAP3K9.

In some embodiments, an irreversible BTK inhibitor for use in the present invention has a percent inhibition comparable to that of a reference kinase inhibitor with respect to one or more kinases selected from the group consisting of c-Kit, PDGFRa, RIPK2, HCK, EPHA6, LYN, CSK, LCK, ZAK/MLTK, LYN B, FRK/PTK5, FYN, RIPK3, BRAF, ARAF and SRMS, or combinations thereof. In some embodiments, the reference kinase inhibitor is Compound 2. In some embodiments, the percent inhibition of the reference kinase inhibitor is that shown for Compound 2 in Example 3.

In some embodiments, the irreversible BTK inhibitor has a percent inhibition comparable to that of a reference kinase inhibitor with respect to the group of kinases consisting of c-Kit, PDGFRa, RIPK2, HCK, EPHA6, LYN, CSK, LCK, ZAK/MLTK, LYN B, FRK/PTK5, FYN, RIPK3, BRAF, ARAF and SRMS, or combinations thereof. In some embodiments, the reference kinase inhibitor is Compound 2. In some embodiments, the percent inhibition of the reference kinase inhibitor is that shown for Compound 2 in Example 3.

In some embodiments, the irreversible BTK inhibitor has a percent inhibition that is not more than the percent inhibition observed for a reference kinase inhibitor with respect to one or more kinases selected from the group consisting of c-Kit, PDGFRa, RIPK2, HCK, EPHA6, LYN, CSK, LCK, ZAK/MLTK, LYN B, FRK/PTK5, FYN, RIPK3, BRAF, ARAF and SRMS, or combinations thereof. In some embodiments, the reference kinase inhibitor is Compound 2. In some embodiments, the percent inhibition of the reference kinase inhibitor is that shown for Compound 2 in Example 3.

In some embodiments, the irreversible BTK inhibitor has not more than about 50%, not more than about 45%, not more than about 40%, not more than about 35%, not more than about 30%, not more than about 25%, not more than about 20%, not more than about 15%, not more than about 10% or not more than about 5% inhibition of a kinase selected from c-Kit, PDGFRa, RIPK2, HCK, EPHA6, LYN, CSK, LCK, ZAK/MLTK, LYN B, FRK/PTK5, FYN, RIPK3, BRAF, ARAF and SRMS, or combinations thereof.

In some embodiments, the irreversible BTK inhibitor has not more than about 50%, not more than about 45%, not more than about 40%, not more than about 35%, not more than about 30%, not more than about 25%, not more than about 20%, not more than about 15%, not more than about 10% or not more than about 5% inhibition of a kinase selected from RIPK2, HCK, LYN, CSK, LCK, LYN B and FYN, or combinations thereof.

In some embodiments, the irreversible BTK inhibitor has not more than about 50%, not more than about 45%, not more than about 40%, not more than about 35%, not more than about 30%, not more than about 25%, not more than about 20%, not more than about 15%, not more than about 10% or not more than about 5% inhibition of a kinase selected from EPHA6, LYN B, FRK/PTK5, RIPK3, BRAF, ARAF and SRMS, or combinations thereof.

In some embodiments, the present invention provides a method of treating, stabilizing or lessening the severity or progression of CLL/SLL comprising administering to a patient in need thereof an irreversible BTK inhibitor in combination with lenalidomide, wherein the irreversible BTK inhibitor has at least about 50% inhibition of a kinase selected from c-Kit, PDGFRa, RIPK2, HCK, EPHA6, LYN, CSK, LCK, ZAK/MLTK, LYN B, FRK/PTK5, FYN, BRAF, RIPK3, ARAF and SRMS, or combinations thereof.

In some embodiments, the irreversible BTK inhibitor has not more than about 50% inhibition of a kinase selected from c-Kit, PDGFRa, RIPK2, HCK, EPHA6, LYN, CSK, LCK, ZAK/MLTK, LYN B, FRK/PTK5, FYN, RIPK3, BRAF, ARAF and SRMS, or combinations thereof. In some embodiments, the irreversible BTK inhibitor has not more than about 50% inhibition of the group of kinases consisting of c-Kit, PDGFRa, RIPK2, HCK, EPHA6, LYN, CSK, LCK, ZAK/MLTK, LYN B, FRK/PTK5, FYN, RIPK3, BRAF, ARAF and SRMS.

In some embodiments, the irreversible BTK inhibitor has not more than about 45% inhibition of a kinase selected from c-Kit, PDGFRa, RIPK2, HCK, EPHA6, LYN, CSK, LCK, ZAK/MLTK, LYN B, FRK/PTK5, FYN, RIPK3, BRAF, ARAF and SRMS, or combinations thereof. In some embodiments, the irreversible BTK inhibitor has not more than about 45% inhibition of the group of kinases consisting of c-Kit, PDGFRa, RIPK2, HCK, EPHA6, LYN, CSK, LCK, ZAK/MLTK, LYN B, FRK/PTK5, FYN, RIPK3, BRAF, ARAF and SRMS.

In some embodiments, the irreversible BTK inhibitor has not more than about 40% inhibition of a kinase selected from c-Kit, PDGFRa, RIPK2, HCK, EPHA6, LYN, CSK, LCK, ZAK/MLTK, LYN B, FRK/PTK5, FYN, RIPK3, BRAF, ARAF and SRMS, or combinations thereof. In some embodiments, the irreversible BTK inhibitor has not more than about 40% inhibition of the group of kinases consisting of c-Kit, PDGFRa, RIPK2, HCK, EPHA6, LYN, CSK, LCK, ZAK/MLTK, LYN B, FRK/PTK5, FYN, RIPK3, BRAF, ARAF and SRMS.

In some embodiments, the irreversible BTK inhibitor has not more than about 35% inhibition of a kinase selected from c-Kit, RIPK2, HCK, EPHA6, LYN, CSK, ZAK/MLTK, LYN B, FRK/PTK5, FYN, RIPK3, BRAF, ARAF and SRMS, or combinations thereof. In some embodiments, the irreversible BTK inhibitor has not more than about 35% inhibition of the group of kinases consisting of c-Kit, RIPK2, HCK, EPHA6, LYN, CSK, ZAK/MLTK, LYN B, FRK/PTK5, FYN, RIPK3, BRAF, ARAF and SRMS.

In some embodiments, the irreversible BTK inhibitor has not more than about 30% inhibition of a kinase selected from c-Kit, RIPK2, HCK, EPHA6, LYN, CSK, ZAK/MLTK, LYN B, FRK/PTK5, FYN, RIPK3, BRAF, ARAF and SRMS, or combinations thereof. In some embodiments, the irreversible BTK inhibitor has not more than about 30% inhibition of the group of kinases consisting of c-Kit, RIPK2, HCK, EPHA6, LYN, CSK, ZAK/MLTK, LYN B, FRK/PTK5, FYN, RIPK3, BRAF, ARAF and SRMS.

In some embodiments, the irreversible BTK inhibitor has not more than about 25% inhibition of a kinase selected from c-Kit, RIPK2, EPHA6, CSK, ZAK/MLTK, LYN B, FRK/PTK5, FYN, RIPK3, BRAF, ARAF and SRMS, or combinations thereof. In some embodiments, the irreversible BTK inhibitor has not more than about 25% inhibition of the group of kinases consisting of c-Kit, IPK2, EPHA6, CSK, ZAK/MLTK, LYN B, FRK/PTK5, FYN, RIPK3, BRAF, ARAF and SRMS.

In some embodiments, the irreversible BTK inhibitor has not more than about 20% inhibition of a kinase selected from EPHA6, CSK, ZAK/MLTK, LYN B, FRK/PTK5, FYN, RIPK3, BRAF, ARAF and SRMS, or combinations thereof. In some embodiments, the irreversible BTK inhibitor has not more than about 20% inhibition of the group of kinases consisting of EPHA6, CSK, ZAK/MLTK, LYN B, FRK/PTK5, FYN, RIPK3, BRAF, ARAF and SRMS.

In some embodiments, the irreversible BTK inhibitor has not more than about 15% inhibition of a kinase selected from EPHA6, LYN B, FRK/PTK5, RIPK3, BRAF, ARAF and SRMS, or combinations thereof. In some embodiments, the irreversible BTK inhibitor has not more than about 15% inhibition of the group of kinases consisting of EPHA6, LYN B, FRK/PTK5, RIPK3, BRAF, ARAF and SRMS.

In some embodiments, the irreversible BTK inhibitor has not more than about 10% inhibition of a kinase selected from EPHA6, LYN B, FRK/PTK5, RIPK3, BRAF, ARAF and SRMS, or combinations thereof. In some embodiments, the irreversible BTK inhibitor has not more than about 10% inhibition of the group of kinases consisting of EPHA6, LYN B, FRK/PTK5, RIPK3, BRAF, ARAF and SRMS.

In some embodiments, the irreversible BTK inhibitor has not more than about 5% inhibition of a kinase selected from EPHA6, FRK/PTK5, RIPK3, BRAF, ARAF and SRMS, or combinations thereof. In some embodiments, the irreversible BTK inhibitor has not more than about 5% inhibition of the group of kinases consisting of EPHA6, FRK/PTK5, RIPK3, BRAF, ARAF and SRMS.

In some embodiments, the irreversible BTK inhibitor has a percent inhibition of LYN comparable to that of a reference kinase inhibitor. In some embodiments, the irreversible BTK inhibitor has a percent inhibition of LYN that is not more than that observed for a reference kinase inhibitor. In some embodiments, the irreversible BTK inhibitor has a percent inhibition of LYN that is about 20-30%. In some embodiments, the irreversible BTK inhibitor has a percent inhibition of LYN that is about 25-30%. In some embodiments, the irreversible BTK inhibitor has a percent inhibition of LYN that is about 25-28%. In some embodiments, the irreversible BTK inhibitor has a percent inhibition of LYN that is not more than about 25%, not more than about 26%, not more than about 27%, not more than about 28%, not more than about 29%, not more than about 30%, not more than about 31%, not more than about 32% or not more than about 33%. In some embodiments, the reference kinase inhibitor is Compound 2. In some embodiments, the percent inhibition of the reference kinase inhibitor is that shown for Compound 2 in Example 3.

In some embodiments, the irreversible BTK inhibitor has a percent inhibition of c-Kit comparable to that of a reference kinase inhibitor. In some embodiments, the irreversible BTK inhibitor has a percent inhibition of c-Kit that is not more than that observed for a reference kinase inhibitor. In some embodiments, the irreversible BTK inhibitor has a percent inhibition of c-Kit that is about 15-25%. In some embodiments, the irreversible BTK inhibitor has a percent inhibition of c-Kit that is about 20-25%. In some embodiments, the irreversible BTK inhibitor has a percent inhibition of c-Kit that is about 20-23%. In some embodiments, the irreversible BTK inhibitor has a percent inhibition of c-Kit that is not more than about 15%, not more than about 16%, not more than about 17%, not more than about 18%, not more than about 19%, not more than about 20%, not more than about 21%, not more than about 22%, not more than about 23%, not more than about 24% or not more than about 25%. In some embodiments, the reference kinase inhibitor is Compound 2. In some embodiments, the percent inhibition of the reference kinase inhibitor is that shown for Compound 2 in Example 3.

In some embodiments, the irreversible BTK inhibitor has a percent inhibition of PDGFRa comparable to that of a reference kinase inhibitor. In some embodiments, the irreversible BTK inhibitor has a percent inhibition of PDGFRa that is not more than that observed for a reference kinase inhibitor. In some embodiments, the irreversible BTK inhibitor has a percent inhibition of PDGFRa that is about 30-40%. In some embodiments, the irreversible BTK inhibitor has a percent inhibition of PDGFRa that is about 35-40%. In some embodiments, the irreversible BTK inhibitor has a percent inhibition of PDGFRa that is about 35-38%. In some embodiments, the irreversible BTK inhibitor has a percent inhibition of PDGFRa that is not more than about 30%, not more than about 31%, not more than about 32%, not more than about 33%, not more than about 34%, not more than about 35%, not more than about 36%, not more than about 37%, not more than about 38%, not more than about 39% or not more than about 40%. In some embodiments, the reference kinase inhibitor is Compound 2. In some embodiments, the percent inhibition of the reference kinase inhibitor is that shown for Compound 2 in Example 3.

In some embodiments, the irreversible BTK inhibitor has a percent inhibition of RIPK2 comparable to that of a reference kinase inhibitor. In some embodiments, the irreversible BTK inhibitor has a percent inhibition of RIPK2 that is not more than that observed for a reference kinase inhibitor. In some embodiments, the irreversible BTK inhibitor has a percent inhibition of RIPK2 that is about 20-30%. In some embodiments, the irreversible BTK inhibitor has a percent inhibition of RIPK2 that is about 20-25%. In some embodiments, the irreversible BTK inhibitor has a percent inhibition of RIPK2 that is about 22-25%. In some embodiments, the irreversible BTK inhibitor has a percent inhibition of RIPK2 that is not more than about 18%, not more than about 19%, not more than about 20%, not more than about 21%, not more than about 22%, not more than about 23%, not more than about 24%, not more than about 25%, not more than about 26% or not more than about 27%. In some embodiments, the reference kinase inhibitor is Compound 2. In some embodiments, the percent inhibition of the reference kinase inhibitor is that shown for Compound 2 in Example 3.

In some embodiments, the irreversible BTK inhibitor has a percent inhibition of HCK comparable to that of a reference kinase inhibitor. In some embodiments, the irreversible BTK inhibitor has a percent inhibition of HCK that is not more than that observed for a reference kinase inhibitor. In some embodiments, the irreversible BTK inhibitor has a percent inhibition of HCK that is about 25-35%. In some embodiments, the irreversible BTK inhibitor has a percent inhibition of HCK that is about 27-32%. In some embodiments, the irreversible BTK inhibitor has a percent inhibition of HCK that is about 28-31%. In some embodiments, the irreversible BTK inhibitor has a percent inhibition of HCK that is not more than about 26%, not more than about 27%, not more than about 28%, not more than about 29%, not more than about 30%, not more than about 31%, not more than about 32%, not more than about 33% or not more than about 34%. In some embodiments, the reference kinase inhibitor is Compound 2. In some embodiments, the percent inhibition of the reference kinase inhibitor is that shown for Compound 2 in Example 3.

In some embodiments, the irreversible BTK inhibitor has a percent inhibition of EPHA6 comparable to that of a reference kinase inhibitor. In some embodiments, the irreversible BTK inhibitor has a percent inhibition of EPHA6 that is not more than that observed for a reference kinase inhibitor. In some embodiments, the irreversible BTK inhibitor has a percent inhibition of EPHA6 that is about 0-10%. In some embodiments, the irreversible BTK inhibitor has a percent inhibition of EPHA6 that is about 0-5%. In some embodiments, the irreversible BTK inhibitor has a percent inhibition of EPHA6 that is about 0-3%. In some embodiments, the irreversible BTK inhibitor has a percent inhibition of EPHA6 that is not more than about 0.5%, not more than about 0.6%, not more than about 0.7%, not more than about 0.8%, not more than about 0.9%, not more than about 1%, not more than about 2%, not more than about 3% or not more than about 4%. In some embodiments, the reference kinase inhibitor is Compound 2. In some embodiments, the percent inhibition of the reference kinase inhibitor is that shown for Compound 2 in Example 3.

In some embodiments, the irreversible BTK inhibitor has a percent inhibition of CSK comparable to that of a reference kinase inhibitor. In some embodiments, the irreversible BTK inhibitor has a percent inhibition of CSK that is not more than that observed for a reference kinase inhibitor. In some embodiments, the irreversible BTK inhibitor has a percent inhibition of CSK that is about 10-20%. In some embodiments, the irreversible BTK inhibitor has a percent inhibition of CSK that is about 15-20%. In some embodiments, the irreversible BTK inhibitor has a percent inhibition of CSK that is about 16-19%. In some embodiments, the irreversible BTK inhibitor has a percent inhibition of CSK that is not more than about 15%, not more than about 16%, not more than about 17%, not more than about 18%, not more than about 19%, not more than about 20%, not more than about 21%, not more than about 22% or not more than about 23%. In some embodiments, the reference kinase inhibitor is Compound 2. In some embodiments, the percent inhibition of the reference kinase inhibitor is that shown for Compound 2 in Example 3.

In some embodiments, the irreversible BTK inhibitor has a percent inhibition of LCK comparable to that of a reference kinase inhibitor. In some embodiments, the irreversible BTK inhibitor has a percent inhibition of LCK that is not more than that observed for a reference kinase inhibitor. In some embodiments, the irreversible BTK inhibitor has a percent inhibition of LCK that is about 30-40%. In some embodiments, the irreversible BTK inhibitor has a percent inhibition of LCK that is about 32-37%. In some embodiments, the irreversible BTK inhibitor has a percent inhibition of LCK that is about 34-37%. In some embodiments, the irreversible BTK inhibitor has a percent inhibition of LCK that is not more than about 34%, not more than about 35%, not more than about 36%, not more than about 37%, not more than about 38%, not more than about 39%, not more than about 40%, not more than about 41% or not more than about 42%. In some embodiments, the reference kinase inhibitor is Compound 2. In some embodiments, the percent inhibition of the reference kinase inhibitor is that shown for Compound 2 in Example 3.

In some embodiments, the irreversible BTK inhibitor has a percent inhibition of ZAK/MLTK comparable to that of a reference kinase inhibitor. In some embodiments, the irreversible BTK inhibitor has a percent inhibition of ZAK/MLTK that is not more than that observed for a reference kinase inhibitor. In some embodiments, the irreversible BTK inhibitor has a percent inhibition of ZAK/MLTK that is about 10-20%. In some embodiments, the irreversible BTK inhibitor has a percent inhibition of ZAK/MLTK that is about 12-17%. In some embodiments, the irreversible BTK inhibitor has a percent inhibition of ZAK/MLTK that is about 14-17%. In some embodiments, the irreversible BTK inhibitor has a percent inhibition of ZAK/MLTK that is not more than about 12%, not more than about 13%, not more than about 14%, not more than about 15%, not more than about 16%, not more than about 17%, not more than about 18%, not more than about 19% or not more than about 20%. In some embodiments, the reference kinase inhibitor is Compound 2. In some embodiments, the percent inhibition of the reference kinase inhibitor is that shown for Compound 2 in Example 3.

In some embodiments, the irreversible BTK inhibitor has a percent inhibition of LYN B comparable to that of a reference kinase inhibitor. In some embodiments, the irreversible BTK inhibitor has a percent inhibition of LYN B that is not more than that observed for a reference kinase inhibitor. In some embodiments, the irreversible BTK inhibitor has a percent inhibition of LYN B that is about 0-10%. In some embodiments, the irreversible BTK inhibitor has a percent inhibition of LYN B that is about 3-8%. In some embodiments, the irreversible BTK inhibitor has a percent inhibition of LYN B that is about 4-7%. In some embodiments, the irreversible BTK inhibitor has a percent inhibition of LYN B that is not more than about 1%, not more than about 2%, not more than about 3%, not more than about 4%, not more than about 5%, not more than about 6%, not more than about 7%, not more than about 8%, not more than about 9% or not more than about 10%. In some embodiments, the reference kinase inhibitor is Compound 2. In some embodiments, the percent inhibition of the reference kinase inhibitor is that shown for Compound 2 in Example 3.

In some embodiments, the irreversible BTK inhibitor has a percent inhibition of FRK/PTK5 comparable to that of a reference kinase inhibitor. In some embodiments, the irreversible BTK inhibitor has a percent inhibition of FRK/PTK5 that is not more than that observed for a reference kinase inhibitor. In some embodiments, the irreversible BTK inhibitor has a percent inhibition of FRK/PTK5 that is about 0-10%. In some embodiments, the irreversible BTK inhibitor has a percent inhibition of FRK/PTK5 that is about 0-5%. In some embodiments, the irreversible BTK inhibitor has a percent inhibition of FRK/PTK5 that is about 0-3%. In some embodiments, the irreversible BTK inhibitor has a percent inhibition of FRK/PTK5 that is not more than about 0.5%, not more than about 0.6%, not more than about 0.7%, not more than about 0.8%, not more than about 0.9%, not more than about 1%, not more than about 1.5%, not more than about 2%, not more than about 3% or not more than about 4%. In some embodiments, the reference kinase inhibitor is Compound 2. In some embodiments, the percent inhibition of the reference kinase inhibitor is that shown for Compound 2 in Example 3.

In some embodiments, the irreversible BTK inhibitor has a percent inhibition of FYN comparable to that of a reference kinase inhibitor. In some embodiments, the irreversible BTK inhibitor has a percent inhibition of FYN that is not more than that observed for a reference kinase inhibitor. In some embodiments, the irreversible BTK inhibitor has a percent inhibition of FYN that is about 15-25%. In some embodiments, the irreversible BTK inhibitor has a percent inhibition of FYN that is about 15-20%. In some embodiments, the irreversible BTK inhibitor has a percent inhibition of FYN that is about 17-20%. In some embodiments, the irreversible BTK inhibitor has a percent inhibition of FYN that is not more than about 15%, not more than about 16%, not more than about 17%, not more than about 18%, not more than about 19%, not more than about 20%, not more than about 21%, not more than about 22% or not more than about 23%. In some embodiments, the reference kinase inhibitor is Compound 2. In some embodiments, the percent inhibition of the reference kinase inhibitor is that shown for Compound 2 in Example 3.

In some embodiments, the irreversible BTK inhibitor has a percent inhibition of BRAF comparable to that of a reference kinase inhibitor. In some embodiments, the irreversible BTK inhibitor has a percent inhibition of BRAF that is not more than that observed for a reference kinase inhibitor. In some embodiments, the irreversible BTK inhibitor has a percent inhibition of BRAF that is about 0-10%. In some embodiments, the irreversible BTK inhibitor has a percent inhibition of BRAF that is about 0.1-5%. In some embodiments, the irreversible BTK inhibitor has a percent inhibition of BRAF that is about 0.2-3%. In some embodiments, the irreversible BTK inhibitor has a percent inhibition of BRAF that is not more than about 0.1%, not more than about 0.2%, not more than about 0.3%, not more than about 0.4%, not more than about 0.5%, not more than about 0.6%, not more than about 0.7%, not more than about 0.8%, not more than about 0.9%, not more than about 1%, not more than about 2%, not more than about 3%, not more than about 4% or not more than about 5%. In some embodiments, the reference kinase inhibitor is Compound 2. In some embodiments, the percent inhibition of the reference kinase inhibitor is that shown for Compound 2 in Example 3.

Compound 1 is an irreversible BTK inhibitor

As described above, Bruton's tyrosine kinase (BTK) is a non-receptor tyrosine kinase with restricted cellular expression largely limited to B-lymphocytes, monocytes, and mast cells or basophils. BTK is a critical component of the B-cell receptor (BCR) signaling network and is crucial for B-cell development. Investigation has revealed that some B-cell malignancies, including non-Hodgkin lymphoma, depend on BCR signaling, suggesting that interruption of such signaling could be a promising therapeutic opportunity. Recently, clinical anti-tumor responses in various B-cell non-Hodgkin lymphoma (NHL) and CLL/SLL have been reported with agents that inhibit spleen tyrosine kinase (SYK) and BTK, both components of the BCR signaling pathway.

Compound 1 is active in a variety of assays and therapeutic models demonstrating covalent, irreversible inhibition of BTK (in enzymatic and cellular assays). Compound 1 inhibits BTK activity by binding with high affinity to the adenosine triphosphate (ATP) binding site of BTK and forming a targeted covalent bond with the BTK protein, providing rapid, complete, and prolonged inhibition of BTK activity, both in vitro and in vivo.

Phosphorylation of the auto-phosphorylation site on BTK (Tyr223) and the BTK responsive site (Tyr1217) on PLCγ2 in Ramos cells, a human Burkitt lymphoma cell line, was inhibited by Compound 1 with an effective concentration required for 50% inhibition (EC₅₀) of 1 nM to 10 nM. Compound 1 demonstrates a high degree of selectivity in cellular assay systems against related kinases.

In single dose studies in healthy subjects, Compound 1 evidenced adequate safety, predictable pharmacokinetics (PK), and, at doses greater than 0.5 mg/kg, 80% to 100% occupancy of the Btk receptor target in normal human peripheral blood B-cells. A phase I dose escalation study of a single agent of Compound 1 is currently being conducted in different B-cell malignancies.

Lenalidomide

Lenalidomide (REVLIMID®) belongs to the class of pharmaceutical compounds known as immunomodulatory drugs (IMiDsP®). The key to its therapeutic potential lies in the fact that it has multiple mechanisms of action, which act to produce both anti-inflammatory and antitumor effects. These effects depend on both the cell type and the triggering stimulus. To date, lenalidomide has been associated with tumor necrosis factor (TNF)-α inhibitory, T cell co-stimulatory, anti-proliferative and anti-angiogenic activities.

Lenalidomide has been approved by multiple global Health Authorities (including the United States Food and Drug Administration, but excluding the European Union) for the treatment of patients with transfusion dependent anemia due to Low- or Intermediate-1-risk myelodysplastic syndrome associated with a deletion 5q cytogenetic abnormality with or without other cytogenetic abnormalities. Lenalidomide has also been approved by multiple global Health Authorities (notably including the US and EU) in combination with dexamethasone, for patients with previously treated multiple myeloma (MM).

Lenalidomide is being investigated as treatment for various oncologic indications, including MM, NHL, and solid tumors. Studies have also been conducted in non-oncologic indications.

Anti-CD20 Antibodies

CD20, the first B-cell specific antigen defined by the monoclonal antibody tositumomab, plays a critical role in B-cell development. Human CD20 is a 297 amino acid (30- to 35-kDa) phosphoprotein with four transmembrane domains encoded by the gene MS4A1 located on chromosome 11q12.2. CD20 plays a critical role in B-cell development and is a biomarker for immunotherapies targeting B-cell derived diseases. CD20 is an integral membrane protein expressed by B lymphocytes in early stages of differentiation and by most B cell lymphomas, but not by differentiated plasma cells. CD20 remains on the membrane of B cells without dissociation or internalization upon antibody binding. CD20 functions though binding to the Src family of tyrosine kinases, such as Lyn, Fyn and Lck, and believed to be involved as a result in the phosphorylation cascade of intracellular proteins. Anti-CD20 antibodies are broadly classified into type I and type II antibodies. Both types of anti-CD 20 antibodies exhibit equal ability in activating Fc-FcγR interactions such as antibody-dependent cellular cytotoxicity (ADCC) and phagocytosis. Type I anti-CD20 antibodies redistribute CD20 into membrane lipid rafts and potently activate complement-dependent cytotoxicity (CDC). Type II anti-CD20 antibodies weakly activate CDC but more potently induce direct programmed cell death.

In some embodiments, the present invention encompasses the recognition that a BTK inhibitor, e.g., Compound 1, together with lenalidomide and an anti-CD20 antibody, is useful in treating BTK-mediated diseases or disorders. Accordingly, in some embodiments, the present invention comprises a method of treating a BTK-mediated disease or disorder, the method comprising administering to a patient in need thereof each of Compound 1, lenalidomide and an anti-CD20 antibody. A person of ordinary skill in the art can readily identify and select additional anti-CD20 antibodies that are useful in the present invention. For example, in some embodiments, such antibodies are described, for example, in U.S. Pat. Nos. 8,153,125, 8,147,832, 8,101,179, 8,084,582, 8,057,793 and 7,879,984, and U.S. Patent Publication Nos. 2011/0129412, 2012/0183545, 2012/0134990 and 2012/0034185.

In some embodiments, an anti-CD20 antibody for use in the present invention is a type I antibody. In some embodiments, an anti-CD20 for use in the present invention is a type II antibody.

In some embodiments, an anti-CD20 antibody is an antibody that binds to a CD20 epitope selected from ¹⁷⁰ANPS¹⁷³ and ¹⁸²YCYSI¹⁸⁵.

In some embodiments, an anti-CD20 antibody has a binding affinity (K_{d}) for an epitope of CD20 of less than 12 nM, less than 11 nM, less than 10 nM, less than 9 nM, less than 8 nM, less than 7 nM, less than 6 nM, less than 5 nM, less than 4 nM, less than 3 nM, less than 2 nM or less than 1 nM.

Rituximab is but one example of an anti-CD20 antibody. In some embodiments, an anti-CD20 antibody for use in the present invention includes, for example, rituximab (Rituxan® or MabThera®), Gazyva® (i.e., obinutuzumab) and Arzerra® (ofatumumab). For ease of reference, provided methods and regimens detailed herein refer to an exemplary anti-CD20 antibody (i.e., rituximab); however, such reference is not intended to limit the present invention to a single anti-CD20 antibody. Indeed, all references to rituximab, or a biosimilar thereof, are to be read by a person skilled in the art to encompass the class of anti-CD20 antibodies. For example, it will be appreciated that the anti-CD20 antibodies ofatumumab (Arzerra®) or obinutuzumab (Gazyva®) can instead be administered in each instance where reference is made to rituximab. Thus, in some embodiments, provided methods comprise administering Compound 1, lenalidomide and/or ofatumumab. In some such embodiments, ofatumumab is administered in 12 doses according to the following schedule: 300 mg initial dose, followed 1 week later by 2000 mg dose weekly for 7 doses, followed 4 weeks later by 2000 mg every 4 weeks for 4 doses. In some embodiments, provided methods comprise administering Compound 1, lenalidomide and/or and obinutuzumab. In some such embodiments, obinutuzumab is administered for six 28-day cycles as follows: 100 mg on day 1, cycle 1; 900 mg on day 2 cycle 1; 1000 mg on days 8 and 15 of cycle 1; and 1000 mg on day 1 of cycles 2-6. Accordingly, in some embodiments, the term “rituximab” encompasses all corresponding anti-CD20 antibodies that fulfill the requirements necessary for obtaining a marketing authorization as an identical or biosimilar product in a country or territory selected from the group of countries consisting of the USA, Europe and Japan.

In some embodiments, an anti-CD20 antibody has the same or similar activity as rituximab, or a biosimilar thereof. In some embodiments, an anti-CD20 antibody binds to the same or similar region or epitope as rituximab or a fragment thereof. In some embodiments, an anti-CD20 antibody competes with the binding of rituximab or a fragment thereof to CD20. In some embodiments, an anti-CD20 antibody is bioequivalent to rituximab or a fragment thereof. In some embodiments, an anti-CD20 antibody is a biosimilar of rituximab or a fragment thereof. In some embodiments, an anti-CD20 antibody is a variant or derivative of rituximab, including functional fragments, derivatives, or antibody conjugates.

Rituximab

Rituximab (Rituxan® or MabThera®) is a genetically engineered cytolytic, chimeric murine/human monoclonal IgG₁ kappa antibody directed against the CD20 cell-surface molecule present in normal B lymphocytes and B-cell CLL and in most forms of non-Hodgkin's B-cell lymphomas. Rituximab has a binding affinity for the CD20 antigen of approximately 8.0 nM. Rituximab can induce complement-dependent cellular cytotoxicity (CDC) and anti-body-dependent cellular cytotoxicity (ADCC), leading to its clinical activity against lymphoma cells. Rituximab can also lead to apoptosis of B cells upon binding to CD20, thereby leading to direct inhibition of cellular growth.

Rituximab is produced by mammalian cell (Chinese Hamster Ovary) suspension culture in a nutrient medium containing the antibiotic gentamicin. Gentamicin is not detectable in the final product. Rituximab is a sterile, clear, colorless, preservative-free liquid concentrate for intravenous administration. Rituximab is supplied at a concentration of 10 mg/mL in either 100 mg/10 mL or 500 mg/50 mL single-use vials. Rituximab is formulated in polysorbate 80 (0.7 mg/mL), sodium citrate dihydrate (7.35 mg/mL), sodium chloride (9 mg/mL) and water for injection. The pH of Rituxan® (or MabThera®) is 6.5

Rituximab is approved for treatment of non-Hodgkin's lymphoma, Wegener's Granulomatosis and Microscopic Polyangiitis. Further, studies have shown that rituximab and bendamustine have also been effective for treatment of relapsed or refractory CLL and NHL, for example, indolent lymphoma and mantle-cell lymphoma. It will be understood and appreciated that when rituximab is administered along with Compound 1 and lenalidomide, rituximab can be used as a single agent or together with bendamustine (“BR”). In some embodiments, provided methods comprise administering each of Compound 1, lenalidomide, rituximab and bendamustine.

### I. General Dosing Protocol

As described herein, provided methods comprise administering two or more therapeutic agents (i.e., Compound 1, lenalidomide and/or an anti-CD20 antibody). It will be appreciated that each of the therapeutic agents can be administered simultaneously or sequentially (e.g., Compound 1 can be administered before, during or after lenalidomide and/or an anti-CD20 antibody and vice versa) as part of a dosing regimen. For example, Compound 1 may be administered one or more hours, days or weeks before administration of either lenalidomide or an anti-CD20 antibody. In other embodiments, each of Compound 1 and lenalidomide may be administered one or more hours, days or weeks before administration of an anti-CD20 antibody. In still other embodiments, each of Compound 1 and an anti-CD20 antibody may be administered one or more hours, days or weeks before administration of lenalidomide.

In some embodiments, the present invention provides methods for treating, stabilizing or lessening the severity or progression of one or more diseases or conditions associated with BTK. In some embodiments, the disease or disorder associated with BTK is a B-cell non-Hodgkin lymphoma. In some such embodiments, the B-cell non-Hodgkin lymphoma is a B-cell non-Hodgkin lymphoma excluding SLL and related disorders such as CLL.

In some embodiments, a B-cell non-Hodgkin lymphoma is selected from diffuse large B-cell lymphoma (DLBCL), follicular lymphoma (FL), mantle cell lymphoma (MCL), mediastinal large B-cell lymphoma (MLBCL), marginal zone B-cell lymphoma (MZL), splenic marginal zone lymphoma (sMZL), Burkitt lymphoma, mucosa-associated lymphatic tissue lymphoma (MALT), intravascular large B-cell lymphoma, primary effusion lymphoma and lymphomatoid granulomatosis.

In some embodiments, the disease or disorder associated with BTK is refractory B-cell non-Hodgkin lymphoma. In some embodiments, the disease or disorder associated with BTK is relapsed B-cell non-Hodgkin lymphoma.

In some embodiments, the B-cell non-Hodgkin lymphoma is selected from aggressive B-cell non-Hodgkin lymphoma or indolent B-cell non-Hodgkin lymphoma. In some embodiments, the B-cell non-Hodgkin lymphoma is aggressive B-cell non-Hodgkin lymphoma. In some such embodiments, the aggressive B-cell non-Hodgkin lymphoma is selected from diffuse large B-cell lymphoma (DLBCL) and Burkitt lymphoma.

In some embodiments, the B-cell non-Hodgkin lymphoma is indolent B-cell non-Hodgkin lymphoma. In some such embodiments, the indolent B-cell non-Hodgkin lymphoma is selected from follicular lymphoma, marginal zone B-cell lymphoma (MZL), splenic marginal zone lymphoma (sMZL) and mucosa-associated lymphatic tissue lymphoma (MALT).

In some embodiments, provided methods comprise administering to a patient in need thereof Compound 1 in combination with lenalidomide. In some such embodiments, each of Compound 1 and lenalidomide is administered as a composition further comprising one or more pharmaceutically acceptable excipients. In some embodiments, provided methods further comprise administering an anti-CD20 antibody, e.g., rituximab. In some embodiments, provided methods comprise administering each of Compound 1, lenalidomide and an anti-CD20 antibody, e.g., rituximab.

In some embodiments, provided methods comprise administering to a patient in need thereof a therapeutically effective amount of Compound 1 in combination with a therapeutically effective amount of lenalidomide. Accordingly, in some embodiments, the present invention provides a method of treating, stabilizing or lessening the severity or progression of a B-cell non-Hodgkin lymphoma, the method comprising administering to a patient in need thereof a therapeutically effective amount of Compound 1 in combination with a therapeutically effective amount of lenalidomide. In some such embodiments, the B-cell non-Hodgkin lymphoma is a B-cell non-Hodgkin lymphoma excluding SLL and related disorders such as CLL. In some embodiments, provided methods further comprise administering an anti-CD20 antibody, e.g., rituximab. In some embodiments, provided methods comprise administering therapeutically effective amounts of each of Compound 1, lenalidomide and an anti-CD20 antibody, e.g., rituximab.

In some embodiments, provided methods comprise administering Compound 1 in combination with lenalidomide, wherein each of Compound 1 and lenalidomide is administered once daily (“QD”). In some embodiments, provided methods comprise administering Compound 1 in combination with lenalidomide, wherein Compound 1 is administered twice daily (“BID”). In some embodiments, provided methods further comprise administering an anti-CD20 antibody, e.g., rituximab. In some embodiments, provided methods comprise administering each of Compound 1, lenalidomide and an anti-CD20 antibody, e.g., rituximab, wherein Compound 1 is administered BID. In some such embodiments, lenalidomide is administered QD. For purposes of clarity, administration of a 375 mg dose of Compound 1 “BID” means that the patient is administered two separate doses of 375 mg in one day.

In some embodiments, provided methods comprise administering Compound 1 in combination with lenalidomide, wherein lenalidomide is administered once daily. In some embodiments, provided methods comprise administering Compound 1 in combination with lenalidomide, wherein Compound 1 is administered twice daily and lenalidomide is administered once daily. In some embodiments, provided methods further comprise administering an anti-CD20 antibody, e.g., rituximab, wherein the anti-CD20 antibody is administered once during a 28-day cycle. In some such embodiments, an anti-CD20 antibody is administered on cycle 1 day 2. In some such embodiments, the anti-CD20 antibody is administered on day 1 of a 28-day cycle. In some embodiments, the anti-CD20 antibody is administered on day 1 of cycles 2-6. In some embodiments, provided methods comprise administering each of Compound 1, lenalidomide and an anti-CD20 antibody, wherein the anti-CD20 antibody is administered once during a 28-day cycle. In some such embodiments, the anti-CD20 antibody is administered on day 1 or day 2 of a 28-day cycle. It is understood that although the methods described herein refer to administering Compound 1, such methods are equally applicable to methods of administering a salt form of Compound 1, e.g., a besylate salt of Compound 1.

In some embodiments, provided methods comprise administering Compound 1 in combination with lenalidomide, wherein lenalidomide is administered once daily on days 1-21 of a 28-day cycle. In some embodiments, provided methods comprise administering Compound 1 in combination with lenalidomide, wherein Compound 1 is administered twice daily on days 1-28 of a 28-day cycle and lenalidomide is administered once daily on days 1-21 of the 28-day cycle. In some embodiments, provided methods comprise administering Compound 1 in combination with lenalidomide, wherein Compound 1 is administered twice daily on days 1-28 of a 28-day cycle and lenalidomide is administered once daily on days 2-22 of the 28-day cycle.

In some embodiments, each of Compound 1 and lenalidomide is administered as pharmaceutically acceptable compositions. In some such embodiments, each pharmaceutically acceptable composition is formulated as an oral dosage form. In some embodiments, such oral dosage forms are capsules. In some embodiments, provided methods further comprise administering a pharmaceutically acceptable composition of an anti-CD20 antibody, e.g., rituximab. In some embodiments, provided methods comprise administering pharmaceutically acceptable compositions of each of Compound 1, lenalidomide and an anti-CD20 antibody, e.g., rituximab. In some such embodiments, the pharmaceutically acceptable composition of an anti-CD20 antibody is formulated as an intravenous composition.

In some embodiments, a pharmaceutically acceptable composition comprising Compound 1 comprises from about 5% to about 60% of Compound 1, or a pharmaceutically acceptable salt thereof, based upon total weight of the composition. In some embodiments, a pharmaceutically acceptable composition comprising Compound 1 comprises from about 5% to about 15% or about 7% to about 15% or about 7% to about 10% or about 9% to about 12% of Compound 1, based upon total weight of the composition. In some embodiments, provided methods comprise administering to a patient in need thereof a pharmaceutically acceptable composition comprising from about 25% to about 75% or about 30% to about 60% or about 40% to about 50% or about 40% to about 45% of Compound 1, based upon total weight of the formulation. In certain embodiments, provided regimens comprise administering to a patient in need thereof a pharmaceutically acceptable composition comprising from about 6%, about 7%, about 8%, about 9%, about 10%, about 11%, about 12%, about 13%, about 20%, about 30%, about 40%, about 41%, about 42%, about 43%, about 44%, about 45%, about 50%, about 60%, about 70%, or about 75% of Compound 1, based upon total weight of given composition or formulation.

In some embodiments, a pharmaceutically acceptable composition comprising lenalidomide comprises from about 2.5% to about 6.25% of lenalidomide, based on the total weight of the composition. In some embodiments, a pharmaceutically acceptable composition comprises from about 2.5%, about 3.75% or about 6.25% of lenalidomide, or a pharmaceutically acceptable salt thereof, based upon total weight of the composition. In some embodiments, the composition of lenalidomide is the composition that is commercially available from Celgene Corporation. In some embodiments, a pharmaceutically acceptable composition of lenalidomide is the composition described in Table 2 or Table 3.

Rituximab is commercially available as a 10 mg/mL solution comprising sodium citrate, polysorbate 80, sodium chloride, sodium hydroxide, hydrochloric acid and water. Commercially available vials comprise either 100 mg/10 mL or 500 mg/50 mL.

In some embodiments, a pharmaceutically acceptable composition comprises from about 1 mg/mL to about 4 mg/mL rituximab. In some embodiments, a pharmaceutically acceptable composition comprises from about 1 mg/mL, about 2 mg/mL, about 3 mg/mL or about 4 mg/mL rituximab. In some embodiments, a pharmaceutically acceptable composition comprises 10 mg/mL.

In some embodiments, provided methods comprise administering Compound 1 in combination with lenalidomide daily for a period of 1, 2, 3, 4, 5, 6, 7, 8, 9, 10, 11, 12, 13, 14, 15, 16, 17, 18, 19, 20, 21, 22, 23, 24, 25, 26, 27 or 28 days. In some embodiments, a treatment regimen comprises at least one 28-day cycle. As used herein, the term “28-day cycle” means that the combination of Compound 1 and lenalidomide is administered to a patient in need thereof for 28 consecutive days. In some embodiments, the combination of Compound 1 and lenalidomide is administered for at least one 28-day cycle. In some embodiments, the combination of Compound 1 and lenalidomide is administered for at least two, at least three, at least four, at least five or at least six 28-day cycles. In some embodiments, the combination of Compound 1 and lenalidomide is administered for at least seven, at least eight, at least nine, at least ten, at least eleven or at least twelve 28-day cycles. In some embodiments, the combination of Compound 1 and lenalidomide is administered for at least thirteen, at least fourteen, at least fifteen, at least sixteen, at least seventeen or at least eighteen 28-day cycles.

In some embodiments, the combination of Compound 1 and lenalidomide is administered for at least eighteen 28-day cycles, and Compound 1 is further administered for at least one additional 28-day cycle. In some embodiments, the combination of Compound 1 and lenalidomide is administered for at least eighteen 28-day cycles, and Compound 1 is further administered for at least two, at least three, at least four, at least five, at least six, at least seven, at least eight, at least nine, at least ten, at least eleven or at least twelve additional 28-day cycles. In some embodiments, the combination of Compound 1 and lenalidomide is administered for at least eighteen 28-day cycles, and Compound 1 is further administered for at least thirteen, at least fourteen, at least fifteen, at least sixteen, at least seventeen, at least eighteen, at least nineteen, at least twenty, at least twenty-one, at least twenty-two, at least twenty-three or at least twenty-four additional 28-day cycles. In some embodiments, the combination of Compound 1 and lenalidomide is administered to a patient for the duration of the patient's life. In some embodiments, the combination of Compound 1 and lenalidomide is administered for at least eighteen 28-day cycles, and Compound 1 is further administered for the duration of the patient's life. In some embodiments, Compound 1 is administered on days 1 through 28 (for example, one dose each day or two doses each day) of each 28-day cycle and lenalidomide is administered on days 1 through 21 (for example, one dose each day) of one or more 28-day cycles. In some embodiments, Compound 1 is administered on days 1 through 28 of one or more 28-day cycles and lenalidomide is administered on days 2 through 22 of one or more 28-day cycles.

In some embodiments, each of Compound 1, lenalidomide and rituximab is administered for at least one 28-day cycle. In some embodiments, each of Compound 1, lenalidomide and rituximab is administered for at least two, at least three, at least four, at least five, or at least six 28-day cycles. In some embodiments, each of Compound 1, lenalidomide and rituximab is administered for at least seven, at least eight, at least nine, at least ten, at least eleven, at least twelve, at least thirteen or at least fourteen 28-day cycles. In some embodiments, each of Compound 1, lenalidomide and rituximab is administered for the duration of the patient's life. In some embodiments, Compound 1 is administered on days 1 through 28 (for example, one dose each day or two doses each day), lenalidomide is administered on days 1 through 21 of one or more 28-day cycles and rituximab is administered on day 1 of a 28-day cycle.

In some embodiments, each of Compound 1 and rituximab is administered for at least one 28-day cycle before administration of lenalidomide. Thus, in some embodiments, each of Compound 1 and rituximab is administered during cycle 1, and each of Compound 1, lenalidomide and rituximab is administered during cycles 2-6. In some such embodiments, each of Compound 1 and lenalidomide is administered for one or more subsequent cycles.

In some embodiments, two adjacent 28-day cycles may be separated by a rest period. Such a rest period may be one, two, three, four, five, six, seven or more days during which the patient is not administered either or both Compound 1 and lenalidomide. In a preferred embodiment, two adjacent 28-day cycles are continuous.

In some embodiments, provided methods comprise administering to a patient in need thereof Compound 1 in combination with lenalidomide, wherein the patient has failed at least one prior therapy. In some embodiments, provided methods comprise administering to a patient in need thereof each of Compound 1, lenalidomide and rituximab, wherein the patient has failed at least one prior therapy.

Unit Dosage Forms

Pharmaceutical compositions for use in the present invention may be prepared as a unit dosage form. A person of ordinary skill will appreciate that the unit dosage forms described herein refer to an amount of a component in its free base form. A person skilled in the art will further appreciate that, when a pharmaceutical composition comprises a salt form of one component, for example, a besylate salt form of Compound 1, the amount of the salt form present in the composition is an amount that is equivalent to a unit dose of the free base of the component (i.e., of Compound 1). For example, a pharmaceutical composition comprising a besylate salt of Compound 1 would contain 34.97 mg of the besylate salt form necessary to deliver an equivalent 25 mg unit dose of the free base of Compound 1.

In some embodiments, provided methods comprise administering to a patient in need thereof a therapeutically effective amount of Compound 1, wherein the therapeutically effective amount of Compound 1 is about 250 mg to about 1250 mg. In some embodiments, the therapeutically effective amount of Compound 1 is administered as one or more discreet doses. For example, in some embodiments, a therapeutically effective amount of Compound 1 is 250 mg, wherein the therapeutically effective amount is administered as 125 mg twice daily (BID). In some embodiments, a therapeutically effective amount of Compound 1 is 500 mg, wherein the therapeutically effective amount is administered as 250 mg twice daily (BID). In some embodiments, a therapeutically effective amount of Compound 1 is 750 mg, wherein the therapeutically effective amount is administered as 375 mg twice daily (BID). In some embodiments, a therapeutically effective amount of Compound 1 is 1000 mg, wherein the therapeutically effective amount is administered as 500 mg twice daily (BID).

In some embodiments, provided methods comprise administering to a patient in need thereof a therapeutically effective amount of Compound 1, wherein the therapeutically effective amount of Compound 1 is about 125 mg to about 1250 mg, or about 125 mg to about 1125 mg, or about 125 mg to about 1000 mg, or about 125 mg to about 875 mg, or about 125 mg to about 750 mg, or about 125 mg to about 625 mg, or about 125 mg to about 500 mg, or about 125 mg to about 375 mg, or about 125 mg to about 250 mg, or about 250 mg to about 1250 mg, or about 250 mg to about 1125 mg, or about 250 mg to about 1000 mg, or about 250 mg to about 875 mg, or about 250 mg to about 750 mg, or about 250 mg to about 625 mg, or about 250 mg to about 500 mg, or about 250 mg to about 375 mg, or about 375 mg to about 1250 mg, or about 375 mg to about 1125 mg, or about 375 mg to about 1000 mg, or about 375 mg to about 875 mg, or about 375 mg to about 750 mg, or about 375 mg to about 625 mg, or about 375 mg to about 500 mg, or about 500 mg to about 1250 mg, or about 500 mg to about 1125 mg, or about 500 mg to about 1000 mg, or about 500 mg to about 875 mg, or about 500 mg to about 750 mg, or about 500 mg to about 625 mg, or about 625 mg to about 1250 mg, or about 625 mg to about 1125 mg, or about 625 mg to about 1000 mg, or about 625 mg to about 875 mg, or about 625 mg to about 750 mg, or about 750 mg to about 1250 mg, or about 750 mg to about 1125 mg, or about 750 mg to about 1000 mg, or about 875 mg to about 1250 mg, or about 875 mg to about 1125 mg, or about 875 mg to about 1000 mg.

In some embodiments, provided methods comprise administering to a patient in need thereof a therapeutically effective amount of Compound 1, wherein the therapeutically effective amount of Compound 1 is about 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, 200 mg, 205 mg, 210 mg, 215 mg, 220 mg, 225 mg, 230 mg, 235 mg, 240 mg, 245 mg, 250 mg, 255 mg, 260 mg, 265 mg, 270 mg, 275 mg, 280 mg, 285 mg, 290 mg, 295 mg, 300 mg, 305 mg, 310 mg, 315 mg, 320 mg, 325 mg, 330 mg, 335 mg, 340 mg, 345 mg, 350 mg, 355 mg, 360 mg, 365 mg, 370 mg, 375 mg, 380 mg, 385 mg, 390 mg, 395 mg, 400 mg, 405 mg, 410 mg, 415 mg, 420 mg, 425 mg, 430 mg, 435 mg, 440 mg, 445 mg, 450 mg, 455 mg, 460 mg, 465 mg, 470 mg, 475 mg, 480 mg, 485 mg, 490 mg, 495 mg, 500 mg, 505 mg, 510 mg, 515 mg, 520 mg, 525 mg, 530 mg, 535 mg, 540 mg, 545 mg, 550 mg, 555 mg, 560 mg, 565 mg, 570 mg, 575 mg, 580 mg, 585 mg, 590 mg, 595 mg, 600 mg, 605 mg, 610 mg, 615 mg, 620 mg, 625 mg, 630 mg, 635 mg, 640 mg, 645 mg, 650 mg, 655 mg, 660 mg, 665 mg, 670 mg, 675 mg, 680 mg, 685 mg, 690 mg, 695 mg, 700 mg, 705 mg, 710 mg, 715 mg, 720 mg, 725 mg, 730 mg, 735 mg, 740 mg, 745 mg, 750 mg, 755 mg, 760 mg, 765 mg, 770 mg, 775 mg, 780 mg, 785 mg, 790 mg, 795 mg, 800 mg, 805 mg, 810 mg, 815 mg, 820 mg, 825 mg, 830 mg, 835 mg, 840 mg, 845 mg, 850 mg, 855 mg, 860 mg, 865 mg, 870 mg, 875 mg, 880 mg, 885 mg, 890 mg, 895 mg, 900 mg, 905 mg, 910 mg, 915 mg, 920 mg, 925 mg, 930 mg, 935 mg, 940 mg, 945 mg, 950 mg, 955 mg, 960 mg, 965 mg, 970 mg, 975 mg, 980 mg, 985 mg, 990 mg, 995 mg, 1000 mg, 1005 mg, 1010 mg, 1015 mg, 1020 mg, 1025 mg, 1030 mg, 1035 mg, 1040 mg, 1045 mg, 1050 mg, 1055 mg, 1060 mg, 1065 mg, 1070 mg, 1075 mg, 1080 mg, 1085 mg, 1090 mg, 1095 mg, 1100 mg, 1105 mg, 1110 mg, 1115 mg, 1120 mg, 1125 mg, 1130 mg, 1135 mg, 1140 mg, 1145 mg, 1150 mg, 1155 mg, 1160 mg, 1165 mg, 1170 mg, 1175 mg, 1180 mg, 1185 mg, 1190 mg, 1195 mg, 1200 mg, 1205 mg, 1210 mg, 1215 mg, 1220 mg, 1225 mg, 1230 mg, 1235 mg, 1240 mg, 1245 mg or 1250 mg.

In some embodiments, provided methods comprise administering to a patient in need thereof a therapeutically effective amount of lenalidomide, wherein the therapeutically effective amount of lenalidomide is about 2.5 mg to about 25 mg.

In some embodiments, provided methods comprise administering to a patient in need thereof a therapeutically effective amount of lenalidomide, wherein the therapeutically effective amount of lenalidomide is about 2.5 mg to about 25 mg, or about 2.5 mg to about 20 mg, or about 2.5 mg to about 15 mg, or about 2.5 mg to about 10 mg, or about 2.5 mg to about 5 mg, or about 5 mg to about 25 mg, or about 5 mg to about 20 mg, or about 5 mg to about 15 mg, or about 5 mg to about 10 mg, or about 10 mg to about 25 mg, or about 10 mg to about 20 mg, or about 10 mg to about 15 mg, or about 15 mg to about 25 mg, or about 15 mg to about 20 mg, or about 20 mg to about 25 mg.

In some embodiments, provided methods comprise administering to a patient in need thereof about 2.5 mg, 5 mg, 10 mg, 15 mg, 20 mg, 25 mg of lenalidomide.

In some embodiments, provided methods comprise administering to a patient in need thereof a pharmaceutical composition comprising a unit dose of Compound 1 in combination with a unit dose of lenalidomide. In some such embodiments, the unit dose of Compound 1 is about 25 mg, about 50 mg, about 75 mg, about 100 mg, about 125 mg, about 150 mg, about 175 mg, about 200 mg, about 225 mg or about 250 mg.

In some embodiments, the unit dose of lenalidomide is about 2.5 mg, about 5 mg, about 10 mg, about 15 mg or about 25 mg.

In some embodiments, provided methods comprise administering to a patient in need thereof a pharmaceutical composition comprising rituximab, wherein rituximab is administered as an infusion at a rate of 50 mg/hr. In some embodiments, the infusion rate of rituximab is increased by 50 mg/hr every 30 minutes, to a maximum of 400 mg/hr. In some embodiments, the infusion rate of rituximab is increased by 100 mg/hr every 30 minutes, to a maximum of 400 mg/hr. Accordingly, in some embodiments, the infusion rate of rituximab is 100 mg/hr. In some embodiments, the infusion rate of rituximab is 150 mg/hr. In some embodiments, the infusion rate of rituximab is 200 mg/hr. In some embodiments, the infusion rate of rituximab is 250 mg/hr. In some embodiments, the infusion rate of rituximab is 300 mg/hr. In some embodiments, the infusion rate of rituximab is 350 mg/hr. In some embodiments, the infusion rate of rituximab is 400 mg/hr.

### II. Uses of Compounds and Pharmaceutically Acceptable Compositions

Compound 1 and compositions described herein are generally useful for the inhibition of protein kinase activity of one or more enzymes. Examples of kinases that are inhibited by Compound 1 and compositions described herein and against which the methods described herein are useful include BTK and other TEC-kinases, including ITK, TEC, BMX and RLK, or a mutant thereof.

Bruton's tyrosine kinase (“BTK”), a member of TEC-kinases, is a key signaling enzyme expressed in B-lymphocytes, monocytes, and mast cells or basophils. BTK plays an essential role in the B-cell signaling pathway linking cell surface B-cell receptor (BCR) stimulation to downstream intracellular responses.

BTK is a key regulator of B-cell development, activation, signaling, and survival (Kurosaki, Curr. Op. Imm., 2000, 276-281; Schaeffer and Schwartzberg, Curr. Op. Imm. 2000, 282-288). In addition, BTK plays a role in a number of other hematopoietic cell signaling pathways, e.g., Toll like receptor (TLR) and cytokine receptor-mediated TNF-α production in macrophages, IgE receptor (Fc_epsilon_RI) signaling in mast cells, inhibition of Fas/APO-1 apoptotic signaling in B-lineage lymphoid cells, and collagen-stimulated platelet aggregation. See, e.g., C. A. Jeffries, et al., (2003), Journal of Biological Chemistry 278:26258-26264; N. J. Horwood, et al., (2003), The Journal of Experimental Medicine 197: 1603-1611; Iwaki et al. (2005), Journal of Biological Chemistry 280(48):40261-40270; Vassilev et al. (1999), Journal of Biological Chemistry 274(3): 1646-1656, and Quek et al. (1998), Current Biology 8(20): 1137-1140.

Patients with inherited inactivating mutations in BTK have a profound block in B-cell development, resulting in the almost complete absence of mature B lymphocytes and plasma cells, severely reduced Ig levels and a profound inhibition of humoral response to recall antigens (reviewed in Vihinen et al Frontiers in Bioscience 5: d917-928). Mice deficient in BTK also have a reduced number of peripheral B-cells and greatly decreased serum levels of IgM and IgG3. BTK deletion in mice has a profound effect on B-cell proliferation induced by anti-IgM, and inhibits immune responses to thymus-independent type II antigens (Ellmeier et al, J Exp Med 192: 1611-1623 (2000)). BTK also plays a crucial role in mast cell activation through the high-affinity IgE receptor (Fc_epsilon_RI). BTK deficient murine mast cells have reduced degranulation and decreased production of proinflammatory cytokines following Fc_epsilon_RI cross-linking (Kawakami et al. Journal of Leukocyte Biology 65: 286-290).

B-Cell Non-Hodgkin Lymphomas

The B-cell non-Hodgkin lymphomas (B-NHL) exhibit variable clinical behavior and are principally classified on the basis of morphologic criteria. Although many specific entities are recognized, the two most prevalent categories comprise diffuse large B-cell lymphomas (DLBCL), accounting for approximately 33% of non-Hodgkin lymphoma, and the follicular B-cell lymphomas, comprising 20-25% of non-Hodgkin lymphoma. Other clinically relevant categories include mantle cell, marginal zone (including the extranodal marginal zone lymphoma of mucosa-associated lymphoid tissue, [MALT]), primary mediastinal large B-cell, and Burkitt lymphomas.

Diffuse large B-cell lymphoma typically presents as an aggressive neoplasm with a median survival of less than 1 year if left untreated. For several decades, multi-agent chemotherapy with cyclophosphamide, doxorubicin, vincristine, and prednisone (CHOP) was the front-line standard treatment. This regimen produced 41% disease-free and 54% overall survival (OS) at 3 years, and was demonstrated to be as effective as more complicated and toxic chemotherapy regimens. Compared to CHOP alone, the current front-line treatment standard of the anti-CD20 monoclonal antibody rituximab administered concurrently with CHOP (R-CHOP) results in superior event-free survival (EFS) and OS in both poor risk older patients (60-80 years of age; 7 year follow-up: EFS 25% (CHOP) vs. 42% (R-CHOP); OS 35% vs. 53%), as well as in good risk younger patients (18-60 years of age; 3 year follow-up: EFS 59% (CHOP) vs. 79% (R-CHOP); OS 84% vs. 93%). A clinical scoring index, the International Prognostic Index (IPI), was developed to stratify patients by prognosis. More recently, gene expression profiling was utilized to distinguish 3 prognostic groups of patients with DLBCL: germinal center B-cell like, activated B-cell like, and a diffuse type 3 group. These 3 molecular subgroups were not strictly related to any specific histologic sub-type of DLBCL, and they predicted survival following anthracycline-based chemotherapy independently of the IPI. Five year OS following anthracycline-based chemotherapy for the germinal center B-cell-like group was 60% compared with 39% for the diffuse type 3 group, and 35% for the activated B-cell-like group. In some embodiments, the present invention provides a method of treating, stabilizing or lessening the severity or progression of DLBCL, wherein the DLBCL is activated B-cell (ABC) DLBCL.

Despite the success of R-CHOP in treating DLBCL, some patients relapse. Based largely on the PARMA study, the standard treatment approach to fit patients with relapsed disease is to employ salvage chemotherapy and consolidation with autologous stem cell transplant (ASCT). In the PARMA trial, the use of transplantation following salvage chemotherapy versus continued salvage chemotherapy alone resulted in improved 5 year EFS (46% vs. 12%) and OS (53% vs. 32%).

The follicular NHLs are characterized by a relatively indolent clinical course and high response rates to various chemotherapies, immunotherapies, radioimmunotherapies, and radiation therapy. Not all cases require immediate treatment and “watch and wait” remains an option for some. Nonetheless, most patients eventually require treatment for clinical complications of progressively bulky tumor and undergo multiple courses of treatment characterized by variable degrees of remission followed by successive progressions at shorter and shorter intervals. Median OS from diagnosis in the pre-rituximab era was 8-10 years, although various clinical prognostic and molecular classifications have identified subsets with distinctly poorer outcomes, including some with only 4-5 year median survival. Monotherapy with rituximab produced clinically meaningful responses in both front-line and relapsed follicular lymphoma; and in randomized studies comparing combinations of alkylator and/or anthracycline-based chemotherapies alone versus with rituximab, the addition of rituximab resulted in improved median progression-free and short-term (3-4 year follow-up) OS. However, despite the introduction of rituximab and other advances in the management of advanced stage follicular lymphomas, these diseases remain largely incurable for the majority of those afflicted.

Indolent NHL is a slow-growing or low-grade form of NHL. Indolent NHL types have a relatively good prognosis with a median survival as long as 10 years, but they usually are not curable in advanced clinical stages. Early stage indolent NHL (stages I and II) has traditionally been treated with radiation therapy. However, a continuous rate of relapse is usually seen in advanced stages of indolent NHL. Accordingly, there remains a need for improved therapies for the treatment of indolent NHL. The present invention provides a method of treating, stabilizing or lessening the severity or progression of indolent Non-Hodgkin lymphoma, wherein said method comprises administering to a patient in need thereof Compound 1 or a pharmaceutically acceptable composition thereof, in combination with lenalidomide.

A person of ordinary skill will appreciate that diseases characterized as “B-cell non-Hodgkin lymphoma” exist as a continuum of diseases or disorders. While the continuum of B-cell non-Hodgkin lymphomas is sometimes discussed in terms of “aggressive” B-cell non-Hodgkin lymphomas or “indolent” B-cell non-Hodgkin lymphomas, a person of ordinary skill will appreciate that a B-cell non-Hodgkin lymphoma characterized as indolent may progress and become an aggressive B-cell non-Hodgkin lymphoma. Conversely, an aggressive form of B-cell non-Hodgkin lymphoma may be downgraded to an indolent or stable form of B-cell non-Hodgkin lymphoma. Reference is made to indolent and aggressive B-cell non-Hodgkin lymphomas as generally understood by a person skilled in the art with the recognition that such characterizations are inherently dynamic and depend on the particular circumstances of the individual.

In some embodiments, a B-cell non-Hodgkin lymphoma is a B-cell non-Hodgkin lymphoma excluding SLL and related disorders such as CLL. In some embodiments, the B-cell non-Hodgkin lymphoma is selected from diffuse large B-cell lymphoma, Burkitt's lymphoma/leukemia, mantle cell lymphoma, mediastinal (thymic) large B-cell lymphoma, follicular lymphoma, marginal zone lymphoma (including extranodal marginal zone B-cell lymphoma and nodal marginal zone B-cell lymphoma) and lymphoplasmacytic lymphoma.

Aggressive B-cell non-Hodgkin lymphomas include diffuse large B-cell lymphoma (DLBCL), Burkitt's lymphoma/leukemia, mantle cell lymphoma and mediastinal lymphoma.

Indolent or slow-growing B-cell non-Hodgkin lymphomas include follicular lymphoma, marginal zone lymphoma and lymphoblastic lymphoma.

Rationale for Targeting Btk and Combinations with Lenalidomide in B-NHL

Significant progress has been achieved in the understanding of the biology of B-cell non-Hodgkin lymphoma and the development of targeted therapies against this disease. Recent preclinical research has shown that Bruton's tyrosine kinase (BTK) is an important signalling protein in lymphomagenesis in DLBCL, FL, marginal zone lymphoma Yang et al., Cancer Cell 2012, 21(6):723-737; Ruiz-Ballesteros et al., Blood 2006, 108(9):3135-3142; Pollan et al., Blood 2005, 106(5):1831-1838. These observations are supported by early reports of clinical trials involving the BTK inhibitor ibrutinib, as well as earlier reports of the activity of a SYK inhibitor, R788, in NHL and CLL subjects. The ABC and GCB types of DLBCL, are being studied. B-cell receptor has also been implicated in the pathogenesis of mantle cell lymphoma.

Thus, based on the critical importance of BCR signaling mediated through Btk for the survival and proliferation of various malignant B-cells; Btk's limited cellular expression in B-cells, macrophages, and monocytes; and demonstrated pre-clinical and early clinical proofs of concept that Btk inhibition produces salutary anti-lymphoma, CLL, and Waldenstrom's macroglobulinemia (WM) effects with acceptable clinical tolerability, targeting Btk with a selective Btk inhibitor is a promising and appropriate therapeutic strategy to investigate further in the clinic.

Compound 1 is an inhibitor of BTK and therefore useful for treating one or more disorders associated with activity of BTK. Compound 1, as its besylate salt, has been shown in recent studies to be safe and effective against B-cell non-Hodgkin lymphoma as a single agent therapeutic. As of Sep. 11, 2012, 9 out of 17 patients with B-cell non-Hodgkin lymphoma have experienced stable disease and continue to on treatment with N-(3-(5-fluoro-2-(4-(2-methoxyethoxyl)phenylamino)pyrimidin-4-ylamino)phenyl)acrylamide besylate monotherapy. Six of eleven evaluable B-NHL patients exhibited decreased lymph node size. See U.S. patent application Ser. No. 13/661,678 and International Patent Application No. PCT/US2012/062133, both filed on Oct. 26, 2012, each of which is hereby incorporated by reference in its entirety. Such data strongly support the use of a BTK inhibitor, and Compound 1 in particular, for treating B-NHL. Compound 1 is generally well tolerated as a single agent at up to 750 mg PO QD and the maximum tolerated dose (MTD) has not yet been reached. Studies are ongoing and additional dose levels currently being investigated include: 1000 mg QD, 1250 mg QD, 375 mg BID and 500 mg BID.

Substantial clinical data further indicate that lenalidomide has activity in relapsed/refractory aggressive B-cell non-Hodgkin lymphoma, possibly with higher activity in the ABC-subtype of DLBCL. Hernandez-Ilizaliturri et al., Cancer 2011, 117(22):5058-2066.

The combination of lenalidomide and a BTK inhibitor has been reported to be synergistic in cell lines and xenograft models. Based on these observations, the combination of Compound 1 and lenalidomide is being investigated in subjects with relapsed or refractory B-cell lymphoma.

In some embodiments, the present invention encompasses the recognition that lenalidomide and Btk inhibition are synergistic in diffuse large B-cell lymphoma (DLBCL) models, perhaps through concomitant inhibition of IkappaB kinase (IKK) and downstream nuclear factor kappa B (NFkB) signaling by both agents. Accordingly, in some embodiments, the present invention encompasses the recognition that a BTK inhibitor such as Compound 1 in combination with lenalidomide is useful in the treatment of B-NHL. In some embodiments, the present invention encompasses the recognition that a BTK inhibitor such as Compound 1, together with lenalidomide and rituximab, is useful in the treatment of B-NHL.

Thus, in some embodiments, the present invention provides a method of treating, stabilizing or lessening the severity or progression of a disorder-cell non-Hodgkin lymphoma, the method comprising the step of administering to a patient in need thereof Compound 1 in combination with lenalidomide. In some such embodiments, the B-cell non-Hodgkin lymphoma is a B-cell non-Hodgkin lymphoma excluding SLL and related disorders such as CLL. In some embodiments, provided methods further comprise administering to a patient in need thereof rituximab. In some embodiments, provided methods comprise administering to a patient in need thereof each of Compound 1, lenalidomide and rituximab. Such methods, dosing regimens and protocols for the administration of said combination are described in further detail, herein.

III. Methods of Treating Diseases or Disorders Associated with Btk

In some embodiments, the present invention provides a method of treating, stabilizing or lessening the severity or progression of a B-cell non-Hodgkin lymphoma, the method comprising administering to a patient in need thereof Compound 1 in combination with lenalidomide. In some such embodiments, the B-cell non-Hodgkin lymphoma is a B-cell non-Hodgkin lymphoma excluding SLL and related disorders such as CLL. In some embodiments, provided methods further comprise administering to a patient in need thereof rituximab. In some embodiments, provided methods comprise administering to a patient in need thereof each of Compound 1, lenalidomide and rituximab.

In some embodiments, the present invention provides a combination of Compound 1 and lenalidomide, wherein the combination demonstrates synergistic effects. In some embodiments, provided methods comprise administering to a patient in need thereof Compound 1 in combination with lenalidomide, wherein the amount(s) of Compound 1 and/or lenalidomide administered are less than the amount administered for one or both compounds when dosed in separate therapeutic regimens. In some embodiments, provided methods further comprise administering to a patient in need thereof rituximab. In some embodiments, provided methods comprise administering to a patient in need thereof each of the therapeutic agents Compound 1, lenalidomide and rituximab wherein such therapeutic agents demonstrate synergistic effects.

In some embodiments, the present invention provides a method of treating, stabilizing or lessening the severity or progression of a B-cell non-Hodgkin lymphoma selected from diffuse large B-cell lymphoma (DLBCL), follicular lymphoma (FL), mantle cell lymphoma (MCL), mediastinal large B-cell lymphoma (MLBCL), marginal zone B-cell lymphoma (MZL), splenic marginal zone lymphoma (sMZL), Burkitt lymphoma, mucosa-associated lymphatic tissue lymphoma (MALT), intravascular large B-cell lymphoma, primary effusion lymphoma and lymphomatoid granulomatosis, the method comprising administering to a patient in need thereof Compound 1 in combination with lenalidomide. In some embodiments, provided methods further comprise administering to a patient in need thereof a composition comprising rituximab. In some embodiments, the present invention provides a method of treating, stabilizing or lessening the severity or progression of a B-cell non-Hodgkin lymphoma selected from diffuse large B-cell lymphoma (DLBCL), follicular lymphoma (FL), mantle cell lymphoma (MCL), mediastinal large B-cell lymphoma (MLBCL), marginal zone B-cell lymphoma (MZL), splenic marginal zone lymphoma (sMZL), Burkitt lymphoma, mucosa-associated lymphatic tissue lymphoma (MALT), intravascular large B-cell lymphoma, primary effusion lymphoma and lymphomatoid granulomatosis, the method comprising administering to a patient in need thereof each of Compound 1, lenalidomide and rituximab.

In some embodiments, provided methods comprise administering to a patient in need thereof a composition comprising Compound 1 in combination with a composition comprising lenalidomide. In some embodiments, provided methods further comprise administering to a patient in need thereof a composition comprising rituximab. In some embodiments, provided methods comprise administering to a patient in need thereof compositions comprising each of Compound 1, lenalidomide and rituximab.

In some embodiments, the composition comprising Compound 1 further comprises one or more pharmaceutically acceptable excipients. In some such embodiments, the composition comprising Compound 1 is formulated as an oral dosage form. In some embodiments, the oral dosage form is a capsule.

In some embodiments, the composition comprising lenalidomide further comprises one or more pharmaceutically acceptable excipients. In some such embodiments, the composition comprising lenalidomide is formulated as an oral dosage form. In some embodiments, the oral dosage form is a capsule.

In some embodiments, the combination of Compound 1 and lenalidomide is administered as a single pharmaceutically acceptable composition.

In some embodiments, provided methods comprise administering to a patient in need thereof a unit dose of Compound 1 in combination with a unit dose of lenalidomide. In some embodiments, provided methods further comprise administering to a patient in need thereof a unit dose of rituximab. In some embodiments, provided methods comprise administering to a patient in need thereof a unit dose of Compound 1, a unit dose of lenalidomide and a unit dose of rituximab. In some embodiments, the unit dose of Compound 1 is about 25 mg, about 50 mg, about 75 mg, about 100 mg, about 125 mg, about 150 mg, about 175 mg, about 200 mg, about 225 mg or about 250 mg. In some embodiments, a unit dose of lenalidomide is about 2.5 mg, about 5 mg, about 10 mg, about 15 mg or about 25 mg.

In some embodiments, provided methods comprise administering to a patient in need thereof a pharmaceutical composition comprising rituximab, wherein rituximab is administered as an infusion at a rate of 50 mg/hr. In some embodiments, the infusion rate of rituximab is increased by 50 mg/hr every 30 minutes, to a maximum of 400 mg/hr. In some embodiments, the infusion rate of rituximab is increased by 100 mg/hr every 30 minutes, to a maximum of 400 mg/hr. Accordingly, in some embodiments, the infusion rate of rituximab is 100 mg/hr. In some embodiments, the infusion rate of rituximab is 150 mg/hr. In some embodiments, the infusion rate of rituximab is 200 mg/hr. In some embodiments, the infusion rate of rituximab is 250 mg/hr. In some embodiments, the infusion rate of rituximab is 300 mg/hr. In some embodiments, the infusion rate of rituximab is 350 mg/hr. In some embodiments, the infusion rate of rituximab is 400 mg/hr.

In some embodiments, the present invention provides a method of treating, stabilizing or lessening the severity or progression of a B-cell non-Hodgkin lymphoma, the method comprising administering to a patient in need thereof Compound 1 in combination with lenalidomide, wherein the patient has failed at least one prior therapy. In some embodiments, provided methods comprise administering to a patient in need thereof each of Compound 1, lenalidomide and rituximab, wherein the patient has failed at least one prior therapy. In some such embodiments, the B-cell non-Hodgkin lymphoma is a B-cell non-Hodgkin lymphoma excluding SLL and related disorders such as CLL.

In some embodiments, provided methods comprise administering to a patient in need thereof about 500 mg to about 1250 mg Compound 1 in combination with about 2.5 mg to about 25 mg lenalidomide. In some embodiments, provided methods comprise administering to a patient in need thereof about 500 mg to about 1250 mg Compound 1, about 2.5 mg to about 25 mg lenalidomide and about 375 mg/m² to about 500 mg/m² rituximab.

In some embodiments, provided methods comprise administering to a patient in need thereof about 750 mg to about 1000 mg Compound 1 and about 15 mg to about 25 mg lenalidomide. In some embodiments, provided methods comprise administering to a patient in need thereof about 750 mg to about 1000 mg Compound 1 and about 15 mg to about 20 mg lenalidomide. In some embodiments, provided methods comprise administering to a patient in need thereof about 750 mg to about 1000 mg Compound 1 and about 20 mg to about 25 mg lenalidomide. In some embodiments, provided methods further comprise administering to a patient in need thereof about 375 mg/m² to about 500 mg/m² rituximab.

In some embodiments, the present invention provides a method of treating, stabilizing or lessening the severity or progression of a B-cell non-Hodgkin lymphoma, the method comprising administering to a patient in need thereof about 125 mg BID to about 500 mg BID Compound 1 in combination with about 5 mg QD to about 25 mg QD lenalidomide. In some embodiments, provided methods comprise administering to a patient in need thereof 375 mg BID to about 500 mg BID Compound 1 in combination with about 15 mg QD to about 25 mg QD lenalidomide. In some embodiments, provided methods further comprise administering to a patient in need thereof about 375 mg/m² to about 500 mg/m² rituximab. In some embodiments, provided methods comprise administering to a patient in need thereof about 125 mg BID to about 500 mg BID Compound 1, about 5 mg QD to about 25 mg QD lenalidomide and about 375 mg/m² to about 500 mg/m² rituximab.

In some embodiments, provided methods comprise administering to a patient in need thereof about 125 mg BID Compound 1 and about 5 mg QD lenalidomide. In some embodiments, provided methods further comprise administering to a patient in need thereof about 375 mg/m² to about 500 mg/m² rituximab.

In some embodiments, provided methods comprise administering to a patient in need thereof about 125 mg BID Compound 1 and about 10 mg QD lenalidomide. In some embodiments, provided methods further comprise administering to a patient in need thereof about 375 mg/m² to about 500 mg/m² rituximab.

In some embodiments, provided methods comprise administering to a patient in need thereof about 125 mg BID Compound 1 and about 15 mg QD lenalidomide. In some embodiments, provided methods further comprise administering to a patient in need thereof about 375 mg/m² to about 500 mg/m² rituximab.

In some embodiments, provided methods comprise administering to a patient in need thereof about 125 mg BID Compound 1 and about 20 mg QD lenalidomide. In some embodiments, provided methods further comprise administering to a patient in need thereof about 375 mg/m² to about 500 mg/m² rituximab.

In some embodiments, provided methods comprise administering to a patient in need thereof about 125 mg BID Compound 1 and about 25 mg QD lenalidomide. In some embodiments, provided methods further comprise administering to a patient in need thereof about 375 mg/m² to about 500 mg/m² rituximab.

In some embodiments, provided methods comprise administering to a patient in need thereof about 250 mg BID Compound 1 and about 5 mg QD lenalidomide. In some embodiments, provided methods further comprise administering to a patient in need thereof about 375 mg/m² to about 500 mg/m² rituximab.

In some embodiments, provided methods comprise administering to a patient in need thereof about 250 mg BID Compound 1 and about 10 mg QD lenalidomide. In some embodiments, provided methods further comprise administering to a patient in need thereof about 375 mg/m² to about 500 mg/m² rituximab.

In some embodiments, provided methods comprise administering to a patient in need thereof about 250 mg BID Compound 1 and about 15 mg QD lenalidomide. In some embodiments, provided methods further comprise administering to a patient in need thereof about 375 mg/m² to about 500 mg/m² rituximab.

In some embodiments, provided methods comprise administering to a patient in need thereof about 250 mg BID Compound 1 and about 20 mg QD lenalidomide. In some embodiments, provided methods further comprise administering to a patient in need thereof about 375 mg/m² to about 500 mg/m² rituximab.

In some embodiments, provided methods comprise administering to a patient in need thereof about 250 mg BID Compound 1 and about 25 mg QD lenalidomide. In some embodiments, provided methods further comprise administering to a patient in need thereof about 375 mg/m² to about 500 mg/m² rituximab.

In some embodiments, provided methods comprise administering to a patient in need thereof about 375 mg BID Compound 1 and about 5 mg QD lenalidomide. In some embodiments, provided methods further comprise administering to a patient in need thereof about 375 mg/m² to about 500 mg/m² rituximab.

In some embodiments, provided methods comprise administering to a patient in need thereof about 375 mg BID Compound 1 and about 10 mg QD lenalidomide. In some embodiments, provided methods further comprise administering to a patient in need thereof about 375 mg/m² to about 500 mg/m² rituximab.

In some embodiments, provided methods comprise administering to a patient in need thereof about 375 mg BID Compound 1 and about 15 mg QD lenalidomide. In some embodiments, provided methods further comprise administering to a patient in need thereof about 375 mg/m² to about 500 mg/m² rituximab.

In some embodiments, provided methods comprise administering to a patient in need thereof about 375 mg BID Compound 1 and about 20 mg QD lenalidomide. In some embodiments, provided methods further comprise administering to a patient in need thereof about 375 mg/m² to about 500 mg/m² rituximab.

In some embodiments, provided methods comprise administering to a patient in need thereof about 375 mg BID Compound 1 and about 25 mg QD lenalidomide. In some embodiments, provided methods further comprise administering to a patient in need thereof about 375 mg/m² to about 500 mg/m² rituximab.

In some embodiments, provided methods comprise administering to a patient in need thereof about 500 mg BID Compound 1 and about 5 mg QD lenalidomide. In some embodiments, provided methods further comprise administering to a patient in need thereof about 375 mg/m² to about 500 mg/m² rituximab.

In some embodiments, provided methods comprise administering to a patient in need thereof about 500 mg BID Compound 1 and about 10 mg QD lenalidomide. In some embodiments, provided methods further comprise administering to a patient in need thereof about 375 mg/m² to about 500 mg/m² rituximab.

In some embodiments, provided methods comprise administering to a patient in need thereof about 500 mg BID Compound 1 and about 15 mg QD lenalidomide. In some embodiments, provided methods further comprise administering to a patient in need thereof about 375 mg/m² to about 500 mg/m² rituximab.

In some embodiments, provided methods comprise administering to a patient in need thereof about 500 mg BID Compound 1 and about 20 mg QD lenalidomide. In some embodiments, provided methods further comprise administering to a patient in need thereof about 375 mg/m² to about 500 mg/m² rituximab.

In some embodiments, provided methods comprise administering to a patient in need thereof about 500 mg BID Compound 1 and about 25 mg QD lenalidomide. In some embodiments, provided methods further comprise administering to a patient in need thereof about 375 mg/m² to about 500 mg/m² rituximab.

In some embodiments, the combination of Compound 1 and lenalidomide is administered over a period of 28 consecutive days (“a 28-day cycle”). In some embodiments, the combination of Compound 1 and lenalidomide is administered for two, three, four, five or six 28-day cycles. In some embodiments, the combination of Compound 1 and lenalidomide is administered for seven, eight, nine, ten, eleven, twelve or more 28-day cycles. In some embodiments, the combination of Compound 1 and lenalidomide is administered for thirteen, fourteen, fifteen, sixteen, seventeen, eighteen or more 28-day cycles. In some embodiments, the combination of Compound 1 and lenalidomide is administered for eighteen 28-day cycles, and Compound 1 is further administered for at least one, at least two, at least three, at least four, at least five, at least six, at least seven, at least eight, at least nine, at least ten, at least eleven, or at least twelve additional 28-day cycles. In some embodiments, the combination of Compound 1 and lenalidomide is administered for eighteen 28-day cycles, and Compound 1 is further administered for at least one, at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, at least twenty, at least twenty-one, at least twenty-two, at least twenty-three or at least twenty-four additional 28-day cycles. In some embodiments, the combination of Compound 1 and lenalidomide is administered for eighteen 28-day cycles, and Compound 1 is further administered to a patient for the duration of the patient's life. In some such embodiments, Compound 1 is administered twice a day on days 1-28 of a 28-day cycle and lenalidomide is administered once a day on days 1-21 of a 28-day cycle. In some embodiments, Compound 1 is administered twice a day on days 1-28 of a 28-day cycle and lenalidomide is administered once a day on days 2-22 of a 28-day cycle. In some embodiments, provided methods further comprise administering to a patient need thereof rituximab on day 1 or day 2 of cycle 1. In some embodiments, provided methods further comprise administering to a patient need thereof rituximab on day 1 of cycles 2, 3, 4, 5 and 6. In some embodiments, provided methods comprise administering to a patient need thereof each of Compound 1, lenalidomide and rituximab, wherein each of Compound 1 and lenalidomide is administered to a patient for one or more 28-day cycles and rituximab is administered on day 1 or 2 of cycles 1, 2, 3, 4, 5 and/or 6. In some embodiments, provided methods comprise administering to a patient need thereof each of Compound 1, lenalidomide and rituximab, wherein Compound 1 is administered twice a day on days 1-28 of a 28-day cycle, lenalidomide is administered once a day on days 1-21 of a 28-day cycle, and rituximab is administered on day 1 of a 28-day cycle.

In some embodiments, each of Compound 1 and rituximab is administered to a patient during one or more cycles, whereafter each of Compound 1, rituximab and lenalidomide is administered to a patient during subsequent cycles. In some embodiments, each of Compound 1 and rituximab is administered to a patient during cycle 1, whereafter each of Compound 1, rituximab and lenalidomide is administered to a patient during subsequent cycles. In some embodiments, provided methods comprise (i) administering each of Compound 1 and rituximab to a patient in need thereof during a first 28-day cycle; and (ii) administering each of Compound 1, rituximab and lenalidomide to the patient during subsequent cycles. In some such embodiments, Compound 1 is administered BID on days 1-28 of each cycle, rituximab is administered on day 1 of each cycle and lenalidomide is administered QD on days 1-21 of the second and subsequent cycles.

In some embodiments, the combination of Compound 1 and lenalidomide is administered to a patient for one or more 28-day cycles, and either of Compound 1 or lenalidomide is further administered to the patient for one or more additional 28-day cycles.

In some embodiments, two adjacent 28-day cycles may be separated by a rest period. Such a rest period may be one, two, three, four, five, six, seven or more days during which the patient is not administered either or both Compound 1 and lenalidomide. In a preferred embodiment, two adjacent 28-day cycles are continuous.

In some embodiments, provided methods comprise administering to a patient in need thereof Compound 1 in combination with lenalidomide, wherein the patient has failed at least one prior therapy.

In some embodiments, the present invention provides a system for treating, stabilizing or lessening the severity of one or more diseases or conditions associated with BTK, the system comprising Compound 1 and lenalidomide. In some embodiments, the system is a kit. In some such embodiments, the kit comprises a pharmaceutical composition comprising Compound 1 and a pharmaceutical composition comprising lenalidomide. In some embodiments, the kit comprises one or more unit doses of Compound 1 in combination with one or more unit doses of lenalidomide. In some embodiments, the kit comprises a pharmaceutical composition comprising Compound 1, a pharmaceutical composition comprising lenalidomide and a pharmaceutical composition comprising rituximab. In some embodiments, the kit comprises one or more unit doses of Compound 1, one or more unit doses of lenalidomide and one or more doses of rituximab. In some embodiments, the kit comprises two 375 mg doses of Compound 1 and one 15 mg dose of lenalidomide. In some embodiments, the kit comprises two 375 mg doses of Compound 1 and one 20 mg dose of lenalidomide. In some embodiments, the kit comprises two 500 mg doses of Compound 1 and one 20 mg dose of lenalidomide. In some embodiments, the kit comprises two 500 mg doses of Compound 1 and one 25 mg dose of lenalidomide.

In some embodiments, a kit comprises seven (7) daily doses of Compound 1 and lenalidomide. In some such embodiments, a kit comprises fourteen (14) 375 mg doses of Compound 1 and seven (7) 15 mg doses of lenalidomide. In some embodiments, a kit comprises fourteen (14) 375 mg doses of Compound 1 and seven (7) 20 mg doses of lenalidomide. In some embodiments, a kit comprises fourteen (14) 500 mg doses of Compound 1 and seven (7) 20 mg doses of lenalidomide. In some embodiments, a kit comprises fourteen (14) 500 mg doses of Compound 1 and seven (7) 25 mg doses of lenalidomide.

In some embodiments, a kit comprises doses of Compound 1 and lenalidomide sufficient for administration during a 28-day cycle. In some embodiments, a kit comprises fifty-six (56) 375 mg doses of Compound 1 and twenty-one (21) 15 mg doses of lenalidomide. In some embodiments, a kit comprises fifty-six (56) 375 mg doses of Compound 1 and twenty-one (21) 20 mg doses of lenalidomide. In some embodiments, a kit comprises fifty-six (56) 500 mg doses of Compound 1 and twenty-one (21) 20 mg doses of lenalidomide. In some embodiments, a kit comprises fifty-six (56) 500 mg doses of Compound 1 and twenty-one (21) 25 mg doses of lenalidomide. In some embodiments, the kit further comprises one 100 mg/10 mL vial of rituximab. In some embodiments, the kit further comprises one 500 mg/50 mL vial of rituximab.

### IV. Formulations Comprising Compound 1

As described above, provided methods comprise administering to a patient in need thereof a pharmaceutically acceptable composition comprising Compound 1, wherein the pharmaceutically acceptable composition is an oral dosage form. In some embodiments, the pharmaceutically acceptable composition is formulated as a capsule.

In certain embodiments, provided methods comprise administering to a patient in need thereof a pharmaceutically acceptable composition which comprises Compound 1, and one or more pharmaceutically acceptable excipients, such as, for example, binders, film coatings, diluents, disintegrants, surfactants (wetting agents), lubricants and glidants (adsorbents), or combinations thereof. One skilled in the art will readily appreciate that the category under which a particular component is listed is not intended to be limiting; in some cases a particular component might appropriately fit in more than one category. Also, as will be appreciated, the same component can sometimes perform different functions, or can perform more than one function, in the context of a particular formulation, for example depending upon the amount of the ingredient and/or the presence of other ingredients and/or active compound(s). In some embodiments, the pharmaceutically acceptable composition is a blended powder.

### i. Binders and Diluents

Pharmaceutical compositions for use in the present invention may comprise one or more binders. Binders are used in the formulation of solid oral dosage forms to hold the active pharmaceutical ingredient and inactive ingredients together in a cohesive mix. In some embodiments, pharmaceutical compositions of the present invention comprise about 5% to about 50% (w/w) of one or more binders and/or diluents. In some embodiments, pharmaceutical compositions of the present invention comprise about 20% (w/w) of one or more binders and/or diluents. Suitable binders and/or diluents (also referred to as “fillers”) are known in the art. Representative binders and/or diluents include, but are not limited to, starches such as celluloses (low molecular weight HPC (hydroxypropyl cellulose), microcrystalline cellulose (e.g., Avicel®), low molecular weight HPMC (hydroxypropyl methylcellulose), low molecular weight carboxymethyl cellulose, ethylcellulose), sugars such as lactose (i.e. lactose monohydrate), sucrose, dextrose, fructose, maltose, glucose, and polyols such as sorbitol, mannitol, lactitol, malitol and xylitol, or a combination thereof. In some embodiments, a provided composition comprises a binder of microcrystalline cellulose and/or lactose monohydrate.

### ii. Disintegrants

Pharmaceutical compositions for use in the present invention may further comprise one or more disintegrants. Suitable disintegrants are known in the art and include, but are not limited to, agar, calcium carbonate, sodium carbonate, sodium bicarbonate, cross-linked sodium carboxymethyl cellulose (croscarmellose sodium), sodium carboxymethyl starch (sodium starch glycolate), microcrystalline cellulose, or a combination thereof. In some embodiments, provided formulations comprise from about 1%, to about 25% disintegrant, based upon total weight of the formulation.

### iii. Surfactants

Surfactants, also referred to as bioavailability enhancers, are well known in the art and typically facilitate drug release and absorption by enhancing the solubility of poorly-soluble drugs. Representative surfactants include, but are not limited to, poloxamers, polyoxyethylene ethers, polyoxyethylene fatty acid esters, polyethylene glycol fatty acid esters, polyoxyethylene hydrogenated castor oil, polyoxyethylene alkyl ether, polysorbates, and combinations thereof. In certain embodiments, the surfactant is a poloxamer. In some such embodiments, the poloxamer is poloxamer 407. In some embodiments, compositions for use in the present invention comprise from about 1% to about 30% by weight of surfactant, based upon total weight of the blended powder.

### iv. Lubricants

Pharmaceutical compositions of the present invention may further comprise one or more lubricants. Lubricants are agents added in small quantities to formulations to improve certain processing characteristics. Lubricants prevent the formulation mixture from sticking to the compression machinery and enhance product flow by reducing interparticulate friction. Representative lubricants include, but are not limited to, magnesium stearate, glyceryl behenate, sodium stearyl fumarate and fatty acids (i.e. palmitic and stearic acids). In certain embodiments, a lubricant is magnesium stearate. In some embodiments, provided formulations comprise from about 0.2% to about 3% lubricant, based upon total weight of given formulation.

### v. Glidants

Pharmaceutical compositions of the present invention may further comprise one or more glidants. Representative glidants include, but are not limited to, silicas (i.e. fumed silica), microcrystalline celluloses, starches (i.e. corn starch) and carbonates (i.e. calcium carbonate and magnesium carbonate). In some embodiments, provided formulations comprise from about 0.2% to about 3% glidant, based upon total weight of given formulation.

### vi. N-(3-(5-fluoro-2-(4-(2-methoxyethoxyl)phenylamino)pyrimidin-4-ylamino)phenyl)acrylamide besylate

As described above, the present invention provides a method of treating a disease or disorder selected from chronic lymphocytic leukemia and small lymphocytic lymphoma, the method comprising administering to a patient in need thereof Compound 1 in combination with lenalidomide. The besylate salt of Compound 1, N-(3-(5-fluoro-2-(4-(2-methoxyethoxyl)phenylamino)pyrimidin-4-ylamino)phenyl)acrylamide benzenesulfonic acid salt, has recently been identified and is currently in clinical trials as monotherapy in subjects with relapsed or refractory B-cell non-Hodgkin lymphoma (B-NHL), chronic lymphocytic leukemia (CLL) and WM. Thus, in some embodiments, provided methods comprise administering to a patient in need thereof a besylate salt of Compound 1.

In some embodiments, provided methods comprise administering to a patient in need thereof a pharmaceutically acceptable composition comprising from about 5% to about 60% of the besylate salt of Compound 1, based upon total weight of the formulation. In some embodiments, provided methods comprise administering to a patient in need thereof a pharmaceutically acceptable composition comprising from about 5% to about 15% or about 7% to about 15% or about 7% to about 10% or about 9% to about 12% of the besylate salt of Compound 1, based upon total weight of the composition. In some embodiments, provided methods comprise administering to a patient in need thereof a pharmaceutically acceptable composition comprising from about 25% to about 75% or about 30% to about 60% or about 40% to about 50% or about 40% to about 45% of the besylate salt of Compound 1, based upon total weight of the formulation. In certain embodiments, provided methods comprise administering to a patient in need thereof a pharmaceutically acceptable composition comprising from about 8%, about 9%, about 10%, about 11%, about 12%, about 13%, about 20%, about 30%, about 40%, about 41%, about 42%, about 43%, about 44%, about 45%, about 50%, about 60%, about 70%, or about 75% of the besylate salt of Compound 1, based upon total weight of given composition or formulation.

In some such embodiments, provided methods comprise administering to a patient in need thereof a pharmaceutical composition comprising a unit dose of Compound 1, wherein Compound 1 is in the form of a besylate salt. In some such embodiments, the unit dose is an amount sufficient to provide about 25 mg, about 50 mg, about 75 mg, about 100 mg, about 125 mg, about 150 mg, about 175 mg, about 200 mg, about 225 mg or about 250 mg of the free base of Compound 1. In some embodiments, the pharmaceutical composition comprising the besylate salt of Compound 1 is a solid oral dosage form.

In some embodiments, the present invention provides a method of treating, stabilizing or lessening the severity or progression of a B-cell non-Hodgkin lymphoma, the method comprising administering to a patient in need thereof Compound 1 in combination with lenalidomide, wherein Compound 1 is administered as the besylate salt. In some such embodiments, the B-cell non-Hodgkin lymphoma is a B-cell non-Hodgkin lymphoma excluding SLL and related disorders such as CLL. In some embodiments, the besylate salt of Compound 1 is administered in the form of a composition comprising one or more pharmaceutically acceptable excipients selected from binders, film coatings, diluents, disintegrants, surfactants, lubricants and glidants. In some embodiments, the present invention provides a method of treating, stabilizing or lessening the severity or progression of a B-cell non-Hodgkin lymphoma, the method comprising administering to a patient in need thereof each of Compound 1, lenalidomide and rituximab, wherein Compound 1 is administered as the besylate salt. In some such embodiments, the besylate salt of Compound 1 is administered in the form of a composition comprising one or more pharmaceutically acceptable excipients selected from binders, film coatings, diluents, disintegrants, surfactants, lubricants and glidants.

In some embodiments, the present invention provides a method of treating, stabilizing or lessening the severity or progression of a B-cell non-Hodgkin lymphoma, the method comprising administering to a patient in need thereof a pharmaceutical composition comprising the besylate salt of Compound 1 in combination with lenalidomide, wherein the amount of besylate salt of Compound 1 is sufficient to deliver about 125 mg, about 250 mg, about 325 mg, about 375 mg, about 400 mg, about 500 mg, about 625 mg, about 750 mg, about 1000 mg or about 1250 mg of the free base of Compound 1. In some such embodiments, the B-cell non-Hodgkin lymphoma is a B-cell non-Hodgkin lymphoma excluding SLL and related disorders such as CLL. In some embodiments, the pharmaceutical composition further comprises one or more pharmaceutically acceptable excipients selected from binders, film coating, diluents, disintegrants, surfactants, lubricants and glidants. In some such embodiments, the pharmaceutical composition comprises one or more pharmaceutically acceptable excipients selected from microcrystalline cellulose, lactose monohydrate, sodium starch, poloxamer 407, fumed silica and magnesium stearate. In some embodiments, the present invention provides a method of treating, stabilizing or lessening the severity or progression of a B-cell non-Hodgkin lymphoma, the method comprising administering to a patient in need thereof a pharmaceutical compositions comprising each of the besylate salt of Compound 1 (i.e., Compound 1 besylate), lenalidomide and rituximab, wherein the amount of besylate salt of Compound 1 is sufficient to deliver about 125 mg, about 250 mg, about 325 mg, about 375 mg, about 400 mg, about 500 mg, about 625 mg, about 750 mg, about 1000 mg or about 1250 mg of the free base of Compound 1.

### V. Process for Preparing Pharmaceutical Compositions Comprising Compound 1

Dry Blend Process:

Milled N-(3-(5-fluoro-2-(4-(2-methoxyethoxyl)phenylamino)pyrimidin-4-ylamino)phenyl)acrylamide besylate, milled microcrystalline cellulose, milled sodium starch glycolate, milled lactose monohydrate, milled poloxamer 407, and sieved fumed silica are weighed and mechanically blended. An intragranular portion of sieved magnesium stearate (2.0%, per Table 1, below) is added to the blender and the formulation blended. This blended formulation is then roller compacted, milled, and then blended. The blended formulation is additionally roller compacted, milled and then blended. The remainder or extragranular portion of the magnesium stearate (0.5%, per Table 1, below) is added and the final formulation is blended. Capsules are either mechanically filled or manually filled via the flood fill method.

All features of each of the aspects of the invention apply to all other aspects mutatis mutandis. Each of the references referred to herein, including but not limited to patents, patent applications and journal articles, is incorporated by reference herein as though fully set forth in its entirety.

In order that the invention described herein may be more fully understood, the following examples are set forth. It should be understood that these examples are for illustrative purposes only and are not to be construed as limiting this invention in any manner.

### EXEMPLIFICATION

### Example 1

### Dose Escalation Study

N-(3-(5-fluoro-2-(4-(2-methoxyethoxyl)phenylamino)pyrimidin-4-ylamino)phenyl)acrylamide besylate is a chemically synthesized small molecule substituted pyrimidine developed as the benzenesulfonic acid salt and is a white to off-white crystalline powder. N-(3-(5-fluoro-2-(4-(2-methoxyethoxyl)phenylamino)pyrimidin-4-ylamino)phenyl)acrylamide besylate is an oral, potent (IC₅₀<0.5 nM) and selective small molecule inhibitor of Btk. N-(3-(5-fluoro-2-(4-(2-methoxyethoxyl)phenylamino)pyrimidin-4-ylamino)phenyl)acrylamide besylate exhibits solubility of approximately 0.16 mg/mL in water and a maximum aqueous solubility of 0.40 mg/mL at approximately pH 3.0. The solubility of N-(3-(5-fluoro-2-(4-(2-methoxyethoxyl)phenylamino)pyrimidin-4-ylamino)phenyl)acrylamide besylate in ethanol is approximately 10 mg/mL. N-(3-(5-fluoro-2-(4-(2-methoxyethoxyl)phenylamino)pyrimidin-4-ylamino)phenyl)acrylamide besylate exhibits no environmental instabilities (i.e. heat, acid, base) that require special handling.

N-(3-(5-fluoro-2-(4-(2-methoxyethoxyl)phenylamino)pyrimidin-4-ylamino)phenyl)acrylamide besylate was formulated into capsules containing the components and quantities listed in Table 1 to obtain the study drug. The capsules listed in Table 1 will be administered during the dose escalation and expansion cohort studies.

TABLE 1
Components of N-(3-(5-fluoro-2-(4-(2-
methoxyethoxy)phenylamino)pyrimidin-4-
ylamino)phenyl)acrylamide besylate capsules

| | Amount per 125 mg | |
|---|---|---|
| Component | Capsule | |
| Capsule shell | 1, size 0 white capsule | |
| N-(3-(5-fluoro-2-(4-(2- | 174.30 | mg |
| methoxyethoxy)phenylamino) | (125 | mg free base) |
| pyrimidin-4-ylamino)phenyl) | | |
| acrylamide besylate | | |
| Microcrystalline cellulose | 101.68 | mg |
| Lactose monohydrate | 41.50 | mg |
| Sodium starch glycolate | 41.50 | mg |
| Poloxamer 407 | 41.50 | mg |
| Fumed silica | 4.15 | mg |
| Magnesium stearate | 10.38 | mg^{‡} |

^{‡}2.0% (8.30 mg) intragranular; 0.5% (2.08 mg) extragranular.

Lenalidomide, also known as 3-(4-amino-1-oxo-1,3-dihydro-2H-isoindol-2-yl)piperidine-2,6-dione, has the following structure:

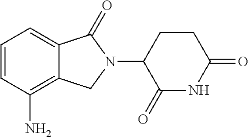 [SMILES: Nc1cccc2c1CN(C1CCC(=O)NC1=O)C2=O]

Lenalidomide is an off-white to pale yellow solid powder and is commercially available as 2.5 mg, 5 mg, 10 mg, 15 mg and 25 mg capsules from Celgene Corporation. Each capsule of lenalidomide contains lactose anhydrous, microcrystalline cellulose, croscarmellose sodium and magnesium stearate.

Lenalidomide will be formulated into capsules containing the components and quantities listed in Table 2 to obtain the study drug. Capsules listed in Table 2 will be administered during the dose escalation and expansion cohort studies according to the dose needed and represent the clinical supply image. As commercial image product may also be used, the components and quantities for commercial image product are listed in Table 3.

TABLE 2
Components of lenalidomide capsules - Clinical Supply Image

| | 2.5 mg | | | | |
|---|---|---|---|---|---|
| | (1%) | 5 mg | 10 mg | 15 mg | 25 mg |
| | Capsule | Capsule | Capsule | Capsule | Capsule |
| Ingredient | Theoretical Weight per Capsule (mg) | | | | |
| Lenalidomide^{a} | 2.5 | 5.0 | 10.0 | 15.0 | 25.0 |
| Lactose | 73.5 | 147.0 | 294.0 | 289.0 | 200.0 |
| Anhydrous^{b} | | | | | |
| Microcrystalline | 20.0 | 40.0 | 80.0 | 80.0 | 159.0 |
| Cellulose | | | | | |
| Croscarmellose | 3.0 | 6.0 | 12.0 | 12.0 | 12.0 |
| Sodium | | | | | |
| Magnesium | 1.0 | 2.0 | 4.0 | 4.0 | 4.0 |
| Stearate | | | | | |
| Total Fill Weight | 100.0 | 200.0 | 400.0 | 400.0 | 400.0 |
| White Capsule | 1 Capsule | — | — | — | — |
| Shells (Size 4) | | | | | |
| White Capsule | | 1 | — | — | — |
| Shells (Size 2) | | Capsule | | | |
| White Capsule | | — | 1 Capsule | 1 | 1 Capsule |
| Shells (Size 0) | | | | Capsule | |

^{a}The weight of lenalidomide is adjusted for assay and water content.
^{b}The weight of Lactose Anhydrous, NF/EP is adjusted to achieve the target total blend weight for the batch.

TABLE 3
Components of lenalidomide capsules-Commercial Supply Image

| | 2.5 mg | 5 mg | 10 mg | 15 mg | 20 mg | 25 mg |
|---|---|---|---|---|---|---|
| | Capsule | Capsule | Capsule | Capsule | Capsule | Capsule |
| Ingredient | Theoretical Weight per Capsule (mg) | | | | | |
| Lenalidomide^{a} | 2.5 | 5.0 | 10.0 | 15.0 | 20.0 | 244.5 |
| Lactose Anhydrous^{b} | 73.5 | 147.0 | 294.0 | 289.0 | 244.5 | 120.5 |
| Microcrystalline | 20.0 | 40.0 | 80.0 | 80.0 | 120.5 | 12.0 |
| Cellulose | | | | | | |
| Croscarmellose | 3.0 | 6.0 | 12.0 | 12.0 | 12.0 | 3.0 |
| Sodium | | | | | | |
| Magnesium Stearate | 1.0 | 2.0 | 4.0 | 4.0 | 3.0 | 400.0 |
| Total Fill Weight | 100.0 | 200.0 | 400.0 | 400.0 | 400.0 | — |
| White body/Blue | 1 Capsule | — | — | — | — | — |
| Green Cap Capsule | | | | | | |
| Shells (Size 4) | | | | | | |
| Imprinted with Black | | | | | | |
| Ink^{c} | | | | | | |
| White Capsule Shells | — | 1 Capsule | — | — | — | |
| (Size 2) Imprinted with | | | | | | |
| Black Ink^{c} | | | | | | |
| Pale Yellow | — | — | 1 Capsule | — | — | |
| Body/Blue Green Cap | | | | | | |
| Capsule Shells (Size 0) | | | | | | |
| Imprinted with Black | | | | | | |
| Ink^{c} | | | | | | |
| White Body/ Powder | — | — | — | 1 Capsule | — | |
| Blue Cap Capsule | | | | | | |
| Shells (Size 0) | | | | | | |
| Imprinted with Black | | | | | | |
| Ink^{c} | | | | | | |
| Blue Body/Blue Green | — | — | — | — | 1 Capsule | |
| Cap Capsule Shells | | | | | | |
| (Size 0) Imprinted with | | | | | | |
| Black Ink^{c} | | | | | | |
| White Capsule Shells | — | — | — | — | — | 1 Capsule |
| (Size 0) Imprinted with | | | | | | |
| Black Ink^{c} | | | | | | |

^{a}The weight of lenalidomide is adjusted for assay and water content.
^{b}The weight of Lactose Anhydrous, NF/EP is adjusted to achieve the target total blend weight for the batch.
^{c}The capsule shells are supplied by Capsugel.

Study Design

Subjects with relapsed or refractory B-cell non-Hodgkin lymphoma, excluding SLL, CLL and Waldenstrom Macroglobulinemia, who failed at least one prior treatment regimen were enrolled in a “3+3” dose escalation and expansion study to determine the Not Tolerated Dose (NTD), the Optimal Biologic Effect dose (OBE) and the Maximum Tolerated Dose (MTD) of the combination of Compound 1 twice daily (BID) and lenalidomide once daily (QD). Approximately 48-60 patients will be enrolled in the study.

Four (4) dose levels were defined and outlined in Table 4.

TABLE 4
Study Dosing Schema for Escalating Dose Portion of Study

| | N-(3-(5-fluoro-2-(4-(2- | |
|---|---|---|
| | methoxyethoxy)phenylamino)pyrimi- | |
| | din-4-ylamino)phenyl) | |
| COHORT | acrylamide besylate | Lenalidomide* |
| 1 | 375 mg BID | 15 mg QD |
| 2 | 375 mg BID | 20 mg QD |
| 3 | 500 mg BID | 15 mg QD |
| 4 | 500 mg BID | 20 mg QD |
| 5 | 500 mg BID | 25 mg QD |

*During cycle 1, lenalidomide will be administered on days 2-22. Lenalidomide will be administered on days 1-21 of each 28-day cycle thereafter.

Study treatment was administered in 28-day cycles at specified dose levels as scheduled until disease progression, unacceptable toxicity, or discontinuation for any other reason. Subjects will continue on the starting dose until the preliminary recommended Phase 2 dose (RP2D) is determined, at which point they can be switched to the preliminary RP2D.

Within each cohort, subjects were treated PO (oral) BID (twice daily) with N-(3-(5-fluoro-2-(4-(2-methoxyethoxyl)phenylamino)pyrimidin-4-ylamino)phenyl) acrylamide besylate in combination with lenalidomide during an initial 28-day treatment cycle and were assessed for safety, tolerability and DLT as well as pharmacokinetic (“PK”), pharmacodynamic (“PD”), and disease response.

In certain instances, the physician-investigator may elect to rest a patient during the study, during which time the patient does not receive treatment. For example, the physician-investigator may elect to rest a patient due to occurrence or recurrence of adverse events. For purposes of clarity, a patient who has been rested is still enrolled in the study until the physician-investigator determines that the patient should not continue treatment, at which time such patients are discontinued from further treatment. In this context, treatment duration refers to the time a patient is enrolled in the study, inclusive of all rest periods, until treatment is discontinued.

The dose level at which a patient is enrolled will be based on which cohort is open at the time of enrollment. For the first cycle only, lenalidomide will be administered on days 2 to and N-(3-(5-fluoro-2-(4-(2-methoxyethoxyl)phenylamino)pyrimidin-4-ylamino)phenyl) acrylamide besylate will be administered on days 1-28 of the 28-day cycle. Thereafter, lenalidomide will be administered on days 1-21 of a given 28-day cycle and N-(3-(5-fluoro-2-(4-(2-methoxyethoxyl)phenylamino)pyrimidin-4-ylamino)phenyl)acrylamide besylate will be administered each day of a given 28-day cycle according to Table 4.

A “complete response” (CR) is defined as the complete disappearance of all detectable clinical evidence of disease. A “partial response” (PR) is defined as the regression of measurable disease and no new sites. “Progressive diease” (PD) is defined as any new lesion or increase by ≧50% of previously involved sites from nadir. “Stable disease” is defined as a failure to attain CR/PR or PD. See Cheson et al., “Revised Response Criteria for Malignant Lymphoma,” Journal of Clinical Oncology 2007, 25(5), 579-586, incorporated by reference in its entirety.

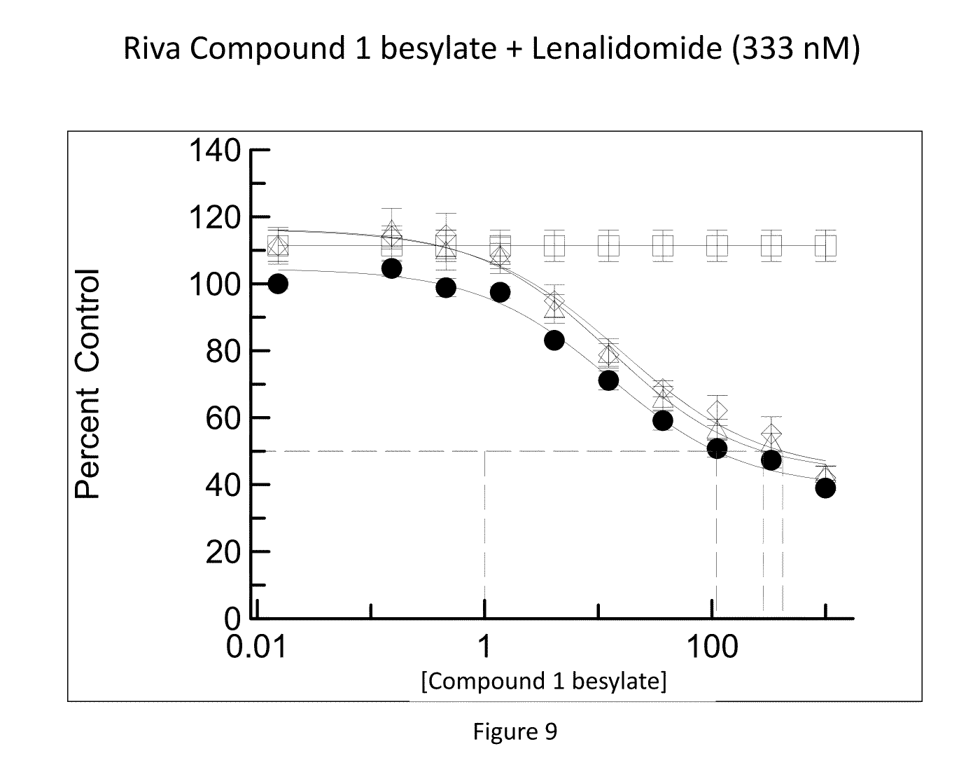

FIG. 9 summarizes the response assessments for patients enrolled in cohort 1. Nine patients were enrolled at dose level 1 and treated with N-(3-(5-fluoro-2-(4-(2-methoxyethoxyl)phenylamino)pyrimidin-4-ylamino)phenyl) acrylamide besylate BID in combination with lenalidomide QD. As of Nov. 12, 2013, one patient with DLBCL exhibited tumor lysis syndrome in the first week of treatment, and the treatment was discontinued after the first 28 day cycle due to progression of disease, without a DLT.

A second patient experienced progressive disease after 12 days, which patient was considered non-evaluable for DLT. A third patient experienced grade 3 diarrhea lasting 12 days and was considered to have experienced a DLT.

Because one of three patients evaluable for DLT experienced a DLT, additional patients were enrolled at dose level 1. A fourth patient did not experience any DLT but disease progression occurred in cycle 2. A fifth patient experienced disease progression during cycle 1 and was considered not evaluable. A sixth patient with DLBCL has completed 2 cycles of treatment without a DLT. A seventh patient with marginal zone lymphoma has completed 2 cycles of treatment without a DLT. An eighth patient had disease progression during cycle 1 and was considered not evaluable for DLT. A ninth patient with DLBCL is currently in cycle 1 of treatment without any DLT thus far.

Preliminary evaluation of patients for tumor response, either by 18-fluorodeoxyglucose positron emission tomography or by physical examination, indicate regression of some tumor lesions in patient 6, patient 7 and patient 9.

A dose level will be considered tolerable if zero (0) of three (3) subjects dosed experience a DLT. Dose escalation, via enrollment in the next higher dose, will be allowed only if none (0) of the first three (3) enrolled subjects experience dose limiting toxicity (DLT). If one (1) of the first three (3) subjects dosed experiences a DLT, three (3) more subjects will be enrolled in that dose cohort. A dose will be considered a NTD when two (2) of six (6) DLT-evaluable subjects in that cohort experience a DLT. A MTD will be declared when at least six (6) subjects have been enrolled and safely complete cycle 1 at that dose level. The MTD is defined as the last dose below the NTD with zero (0) or one (1) DLT-evaluable subjects experiencing DLT during the first 28-day cycle.

During the dose escalation phase, a decision to enroll the next higher dose cohort will be based on review of safety and DLT-evaluable patients.

Subjects will remain on study until the subject discontinues due to disease progression, unacceptable toxicity, withdrawal of consent or any other reason determined by the physician. Preliminary evidence of efficacy will be evaluated.

Patients enrolled in the dose escalation cohorts may escalate to the next higher dose level after six (6) cycles of therapy if at that time the MTD has not been reached with the three or six patients treated at that dose level.

Expansion Cohorts.

After completion of observation for DLTs in the dose escalation study, the accumulated safety, PK, and PD data will be evaluated to select a preliminary RP2D. The preliminary RP2D will be evaluated in three expansion cohorts for a more complete safety profile and further preliminary evaluation of efficacy.

The three (3) expanded cohorts of 12 subjects each are defined as follows:
Cohort A: patients with diffuse large B-cell non-Hodgkin lymphoma
Cohort B: patients with mantle cell lymphoma
Cohort C: patients with any other type of B-cell non-Hodgkin lymphomas except CLL, SLL and Waldenstrom Macroglobulinemia

In certain instances, the physician-investigator may elect to rest a patient during the study, during which time the patient does not receive treatment. For example, the physician-investigator may elect to rest a patient due to occurrence or recurrence of adverse events. For purposes of clarity, a patient who has been rested is still enrolled in the study until the physician-investigator determines that the patient should not continue treatment, at which time such patients are discontinued from further treatment. In this context, treatment duration refers to the time a patient is enrolled in the study, inclusive of all rest periods, until treatment is discontinued.

Adverse Events.

For all cohorts, dose limiting toxicities (DLTs) are defined as specified adverse events (AEs) that are observed within the first 28-day cycle and deemed to be related to treatment. Hematologic DLTs include Grade 3 neutropenia lasting for 7 days or longer or associated with a fever; Grade 4 neutropenia lasting for more than 3 days; Grade 3 thrombocytopenia (platelet count of less than 50,000 cells/mm³ for longer than 7 days); Grade 4 thrombocytopenia lasting more than 3 days; and Grade 4 anemia. Non-hematologic DLTs include Grade 4 or higher non-hematologic AEs of any duration during cycle 1; Grade 3 total bilirubin elevation, whether symptomatic or asymptomatic; and any Grade 3 non-hematologic toxicity except nausea, vomiting and diarrhea lasting less than 24 hours following medical therapy; tumor lysis syndrome which does not progress to Grade 4 and resolves in less than 7 days with medical management; and transient, and Grade 3 non-hematologic laboratory anomaly that is asymptomatic and rapidly reversible (returns to baseline or <Grade 1 within 7 days).

Subjects without disease progression and without DLT at the end of the first 28-day cycle of treatment will be eligible to continue receiving N-(3-(5-fluoro-2-(4-(2-methoxyethoxyl)phenylamino)pyrimidin-4-ylamino)phenyl)acrylamide besylate and lenalidomide for additional 28-day cycles until (i) the patient experiences unacceptable toxicity, (ii) the underlying malignancy progresses, (iii) the patient withdraws consent, or (iv) the treating physician-investigator otherwise determines that the patient should not continue treatment.

Btk Occupancy.

The covalent mechanism of action of N-(3-(5-fluoro-2-(4-(2-methoxyethoxyl)phenylamino)pyrimidin-4-ylamino)phenyl)acrylamide besylate allows for development of a covalent probe to detect free, uninhibited Btk in lysates derived from tissue culture, animal tissues, or clinical samples. PBMC lysates are isolated from whole blood samples 30 minutes before dosing, 4 hours or 24 hours post-dose and incubated with the biotinylated covalent probe. Uninhibited Btk is captured by the covalent probe and quantitated by ELISA. Normalization to untreated control sample allows for determination of the % Btk occupancy.

Btk Target Site Occupancy ELISA:

Cell lysates or spleen homogenates are incubated with 1 μM N¹-(3-(3-(4-(3-acrylamidophenylamino)-5-methylpyrimidin-2-ylamino)phenoxy)propyl)-N⁵-(15-oxo-19-((3 aS,4S,6aR)-2-oxohexahydro-1H-thieno[3,4-d]imidazol-4-yl)-4,7,10-trioxa-14-azanonadecyl)glutaramide (2) in a PBS, 0.05% Tween-20, 1% BSA solution for 1 h at room temperature. Compound 2 has the following structure:

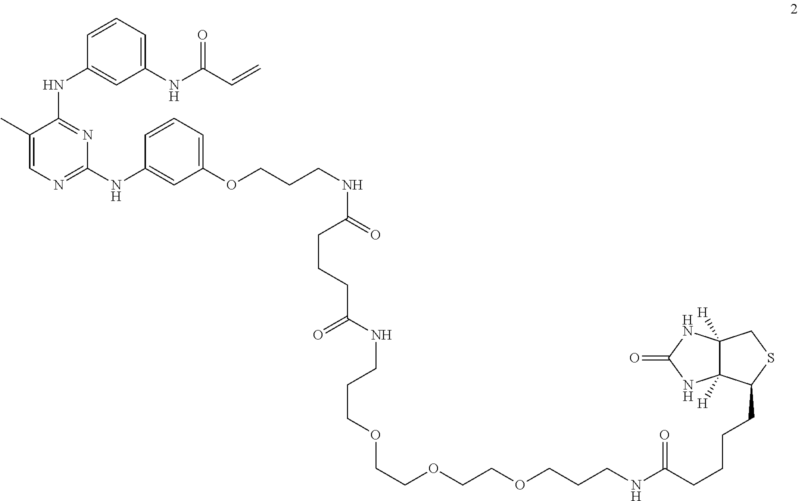 [SMILES: C=CC(=O)Nc1cccc(Nc2nc(Nc3cccc(OCCCNC(=O)CCCC(=O)NCCCOCCOCCOCCCNC(=O)CCCC[C@@H]4SC[C@@H]5NC(=O)N[C@@H]54)c3)ncc2C)c1]

Standards and samples are transferred to a streptavidin-coated 96-well ELISA plate and mixed while shaking for 1 h at room temperature. The α-Btk antibody (BD 611116, 1:1000 dilution in PBS+0.05% Tween-20+0.5% BSA) is then incubated for 1 h at room temperature. After wash, goat α-mouse-HRP (1:5000 dilution in PBS+0.05% Tween-20+0.5% BSA) is added and incubated for 1 h at room temperature. The ELISA is developed with addition of tetramethyl benzidine (TMB) followed by Stop Solution and read at OD 450 nm. The standard curve (11.7-3000 pg/μL) is generated with human full-length recombinant Btk protein and plotted using a 4 parameter curve fit in Gen5 software. Uninhibited Btk detected from samples is normalized to μg total protein as determined by BCA protein analysis (Pierce Cat. 23225).

### Example 2

### Cell Titer Glo Combination Assay

The combination of N-(3-(5-fluoro-2-(4-(2-methoxyethoxyl)phenylamino)pyrimidin-4-ylamino)phenyl)acrylamide besylate (Compound 1 besylate) and lenalidomide was assayed in four model B-cell cancer cell lines to ascertain whether the combination exhibited any synergistic effects.

DLBCL cell lines OCI-LY-10, WSU-DLCL2, Riva and TMD-8 were grown in RMPI+10% FBS medium and plated at 10000 cells/well in 96 well plates. Each cell line is plated in multiple 96 well plates at 90 μL/well.

Based on an initial compound viability assay for each cell line, the range of compound concentration used for each cell line was determined so that maximum and minimum cell viability were achieved with an even spread of cell viability. A stock solution of Compound 1 besylate in DMSO was prepared at 10 mM. The 10 mM stock solution was diluted 3×-10× to between 3333 and 1000 nM and further diluted 3-fold in a 10 point dilution series in DMSO. The dilution series was diluted 50× in RMPI+10% FBS media and 5 μL of Compound 1 besylate was added to 95 μL of cell culture, resulting in a 1:1000 dilution.

Lenalidomide was prepared as a 10 mM stock solution in DMSO and diluted in DMSO to 1000× of the desired final concentration. The dilution series was diluted 200× in RMPI+10% FBS media and 5 μL of lenalidomide was added to 95 μL of cell culture, resulting in a 1:1000 dilution. The final concentration of lenalidomide ranged from 41-10000 nM.

For the combination assays, one concentration of lenalidomide was added to a triplicate dilution series of Compound 1 besylate. At least three, and up to five, different concentrations of lenalidomide were assayed in combination with a triplicate dilution series of Compound 1 besylate. Compound 1 besylate and lenalidomide were applied concomitantly to the cells the day after plating.

72 hours after drug administration, the number of viable cells was determined using CellTiter-Glo according to manufacturer's instructions. Briefly, cells and CellTiter-Glo are equilibrated to room temperature. CellTiter-Glo is added to the cells 1:1 (v/v) and the assay was performed. Results were read on a Molecular Devices spectramax L luminescence detector.

Luminescence readings were normalized as a percent of the average control (DMSO treated) luminescence reading. The expected combination viability was calculated using the formula:

(Compound 1 besylate % survival fraction)×(lenalidomide % survival fraction)/100

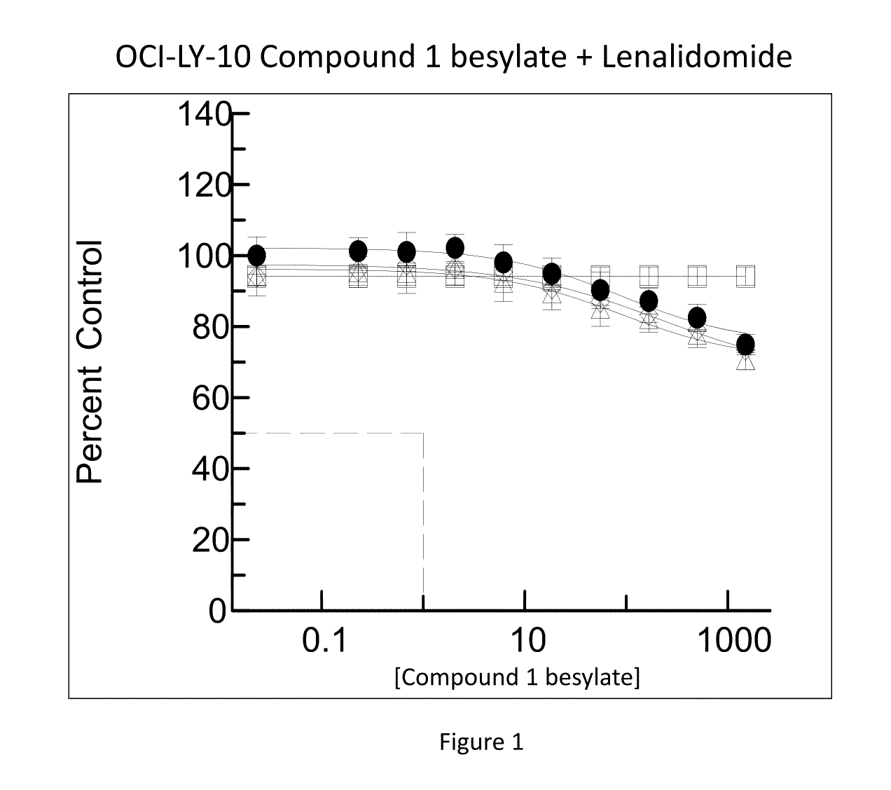

Microsoft Excel with the XLfit plugin was used to plot the viability curves. Synergy (“volcano”) plots were created by plotting the difference between observed and expected combination treatment values. Results are presented in FIGS. 1-12. The graphs depicted in the Figures are exemplary unless otherwise indicated.

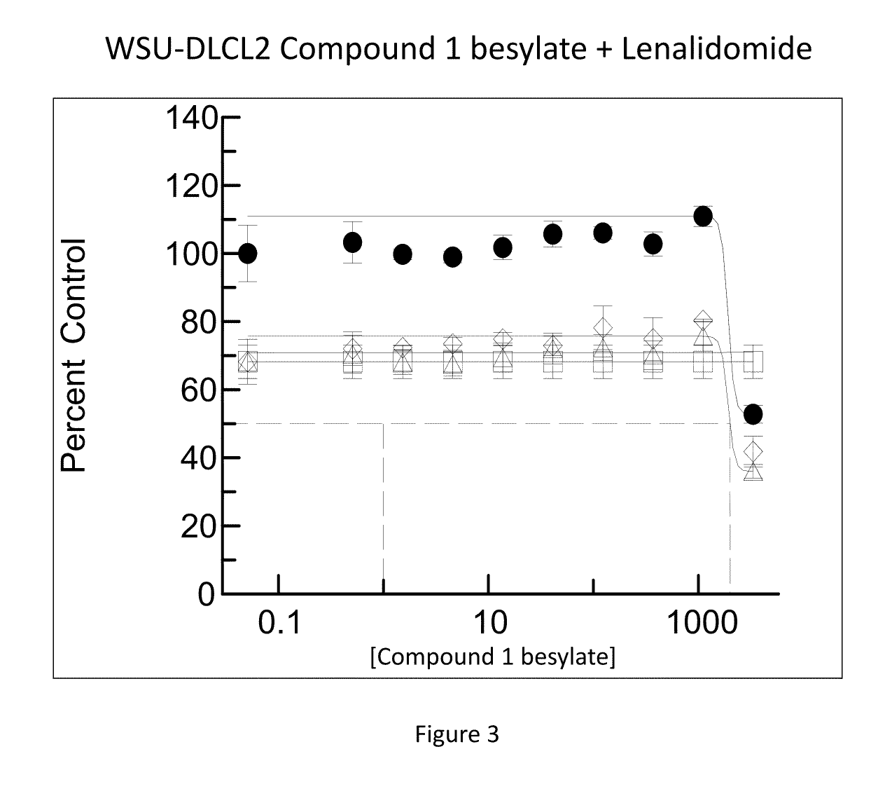

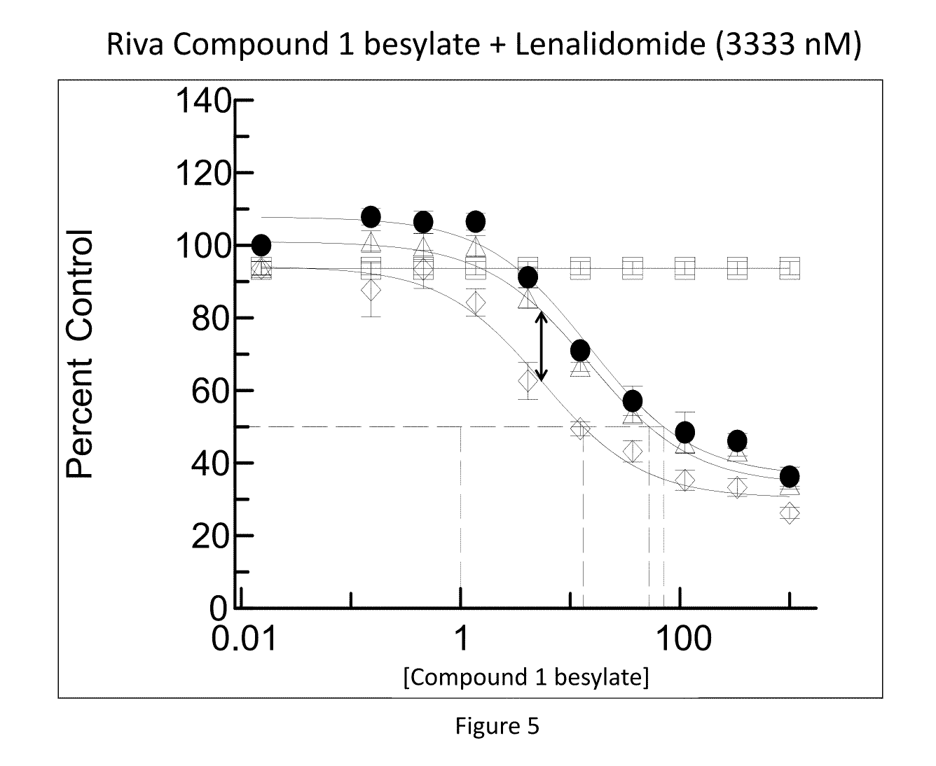

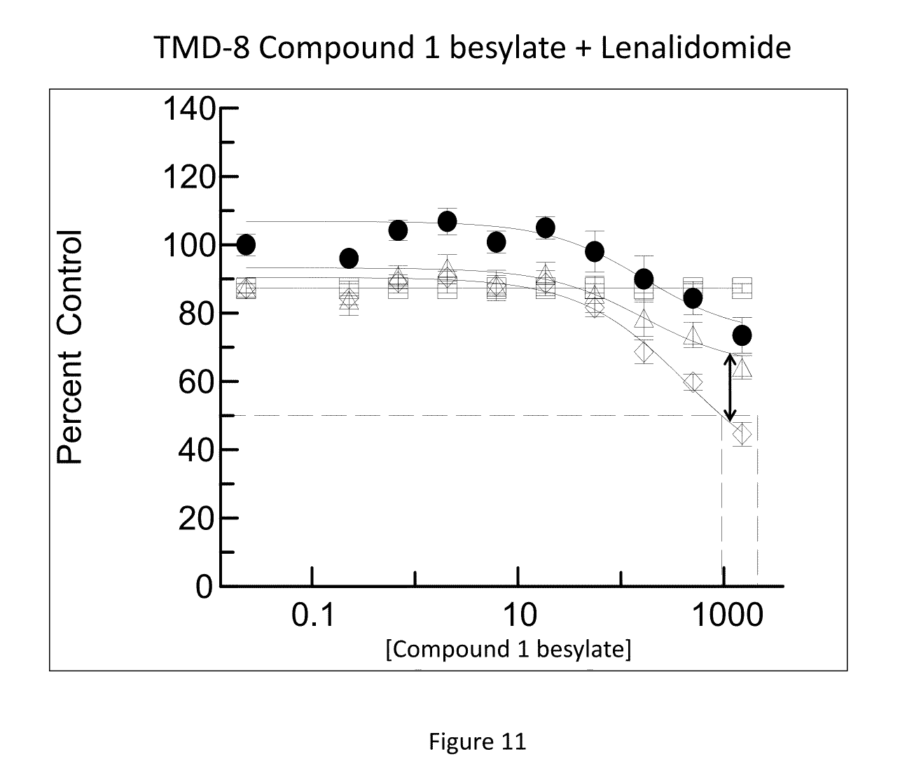

The dose response curves in FIGS. 1, 3, 5, 7, 9, and 11 depict the results of the combination of Compound 1 besylate and lenalidomide in cell lines OCI-LY-10 (FIG. 1), WSU-DLCL2 (FIG. 3), Riva (FIGS. 5, 7 and 9) and TMD-8 (FIG. 11). The response of cells to increasing concentrations of Compound 1 besylate is indicated by . The response of cells to a test concentration of lenalidomide is indicated by □. The expected (or calculated) activity of the combination is indicated by Δ. The observed activity of the combination is indicated by ⋄. The combination is additive when the expected or calculated activity of the combination is equal to the observed activity (i.e., when the lines represented by Δ and ⋄ overlap).

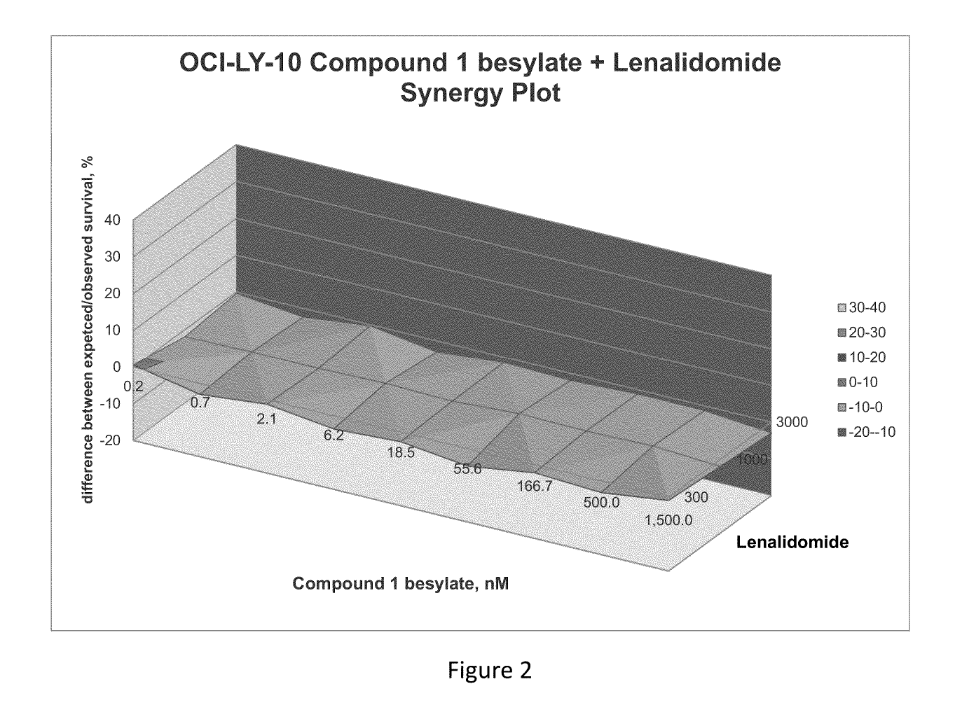

FIG. 1 presents a dose response curve that was observed with Compound 1 besylate and one particular concentration of lenalidomide (3000 nM) in the OCI-LY-10 cell line. The results presented in FIG. 1 appear to show an additive effect, and were confirmed by an additional experiment (data not shown). FIG. 1 therefore can be considered to be representative of data obtained for the combination of Compound 1 besylate and lenalidomide in the OCI-LY-10 cell line. FIG. 2 presents a “volcano” plot of Compound 1 besylate and lenalidomide in the OCI-LY-10 cell line. The volcano plot shown in FIG. 2 was generated using data from experiments run with different concentrations of lenalidomide and is representative of additional experiments in the OCI-LY-10 cell line. The results were confirmed by an additional experiment (data not shown). Compound 1 besylate and lenalidomide thus appear to be additive in the OCI-LY-10 cell line.

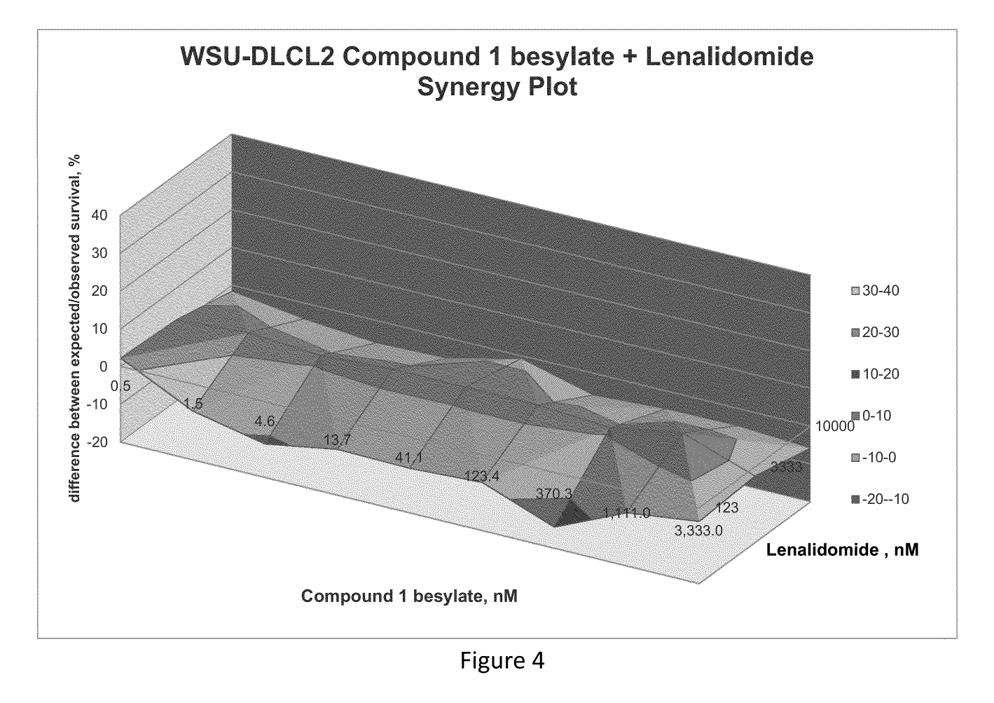

FIG. 3 depicts a particular dose response curve for Compound 1 besylate and one particular concentration of lenalidomide (3333 nM) in the WSU-DLCL2 cell line. The results depicted in FIG. 3 were confirmed by an additional experiment (data not shown). FIG. 3 can thus be considered to be representative of the data obtained for the combination of Compound 1 besylate and lenalidomide in the WSU-DLCL2 cell line. The results presented in FIG. 3 appear to show an additive effect. FIG. 4 presents a “volcano” plot of Compound 1 besylate and lenalidomide in the WSU-DLCL2 cell line. Lenalidomide was also tested in combination with Compound 1 besylate at 37 nM, 111 nM, 370 nM and 1111 nM. No synergy was apparent in this experiment at these concentrations (data not shown). Compound 1 besylate and lenalidomide thus appear to be additive in the WSU-DLCL2 cell line.

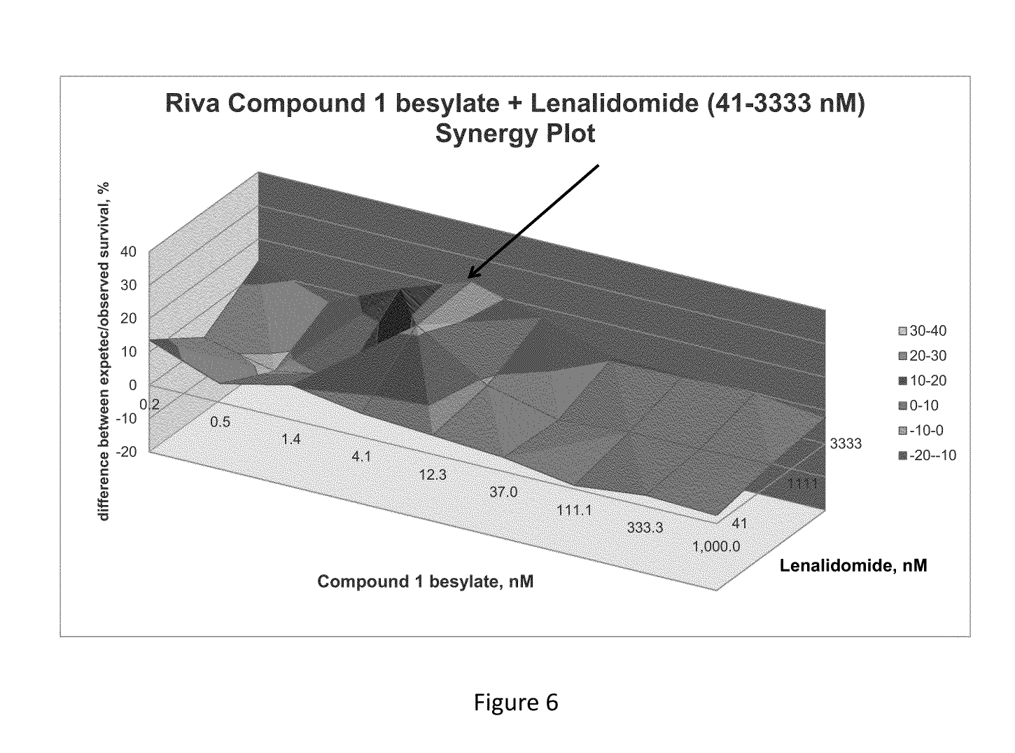

FIG. 5 depicts a particular dose response curve for Compound 1 besylate and lenalidomide (3333 nM) in the Riva cell line. At this concentration, Compound 1 besylate and lenalidomide appear synergistic (indicated by the arrow). FIG. 6 presents a “volcano” plot of Compound 1 besylate and lenalidomide in the Riva cell line at lenalidomide concentrations of 41 nM, 1111 nM and 3333 nM. At certain concentrations, Compound 1 besylate and lenalidomide appear synergistic (indicated by the arrow).

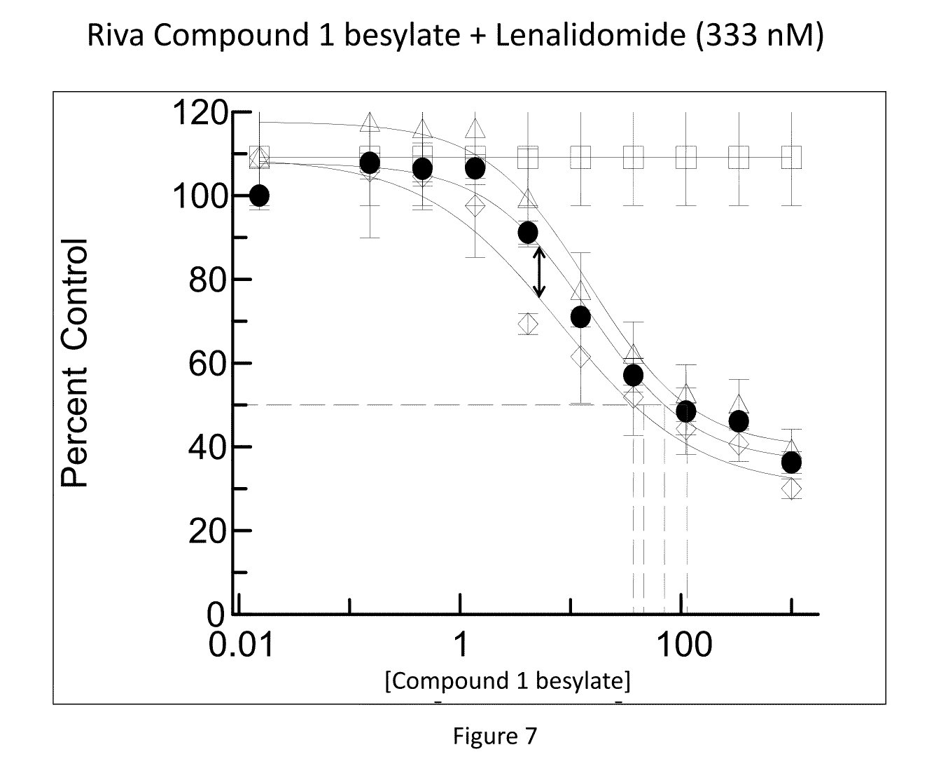

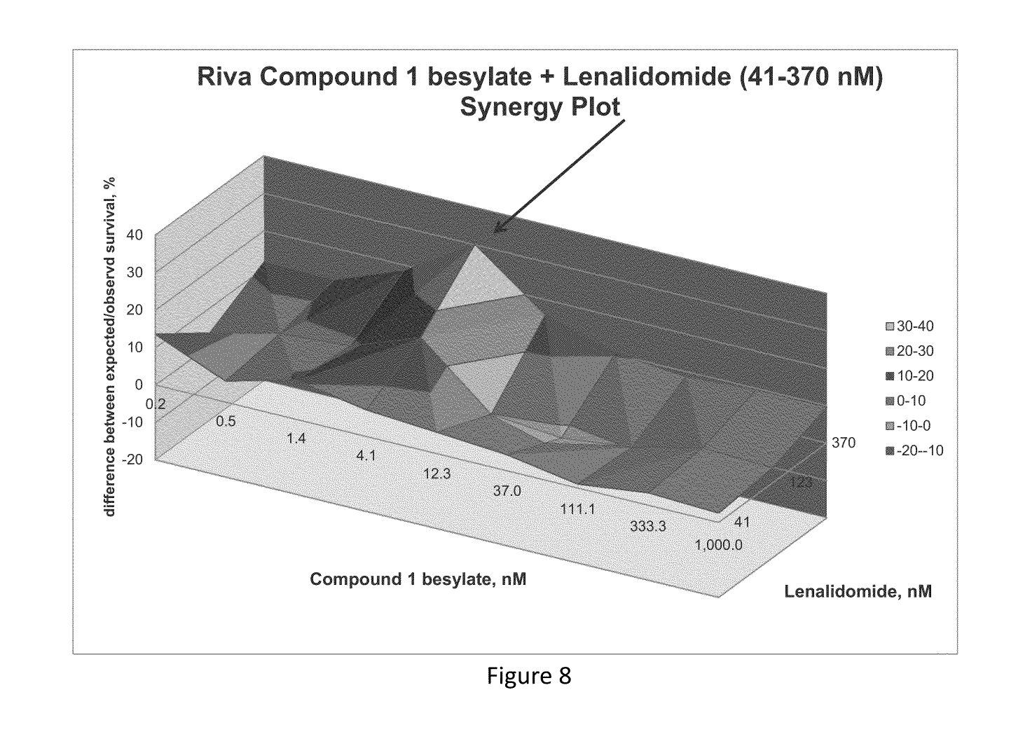

FIG. 7 presents a particular dose response curve for Compound 1 besylate and lenalidomide (333 nM) in the Riva cell line. Compound 1 besylate and lenalidomide in this Figure appear to be synergistic (indicated by the arrow). FIG. 8 presents a “volcano” plot of a particular experiment with Compound 1 besylate and lenalidomide in the Riva cell line at lenalidomide concentrations of 41 nM, 123 nM and 370 nM. Compound 1 besylate and lenalidomide in this Figure appear to be synergistic (indicated by the arrow).

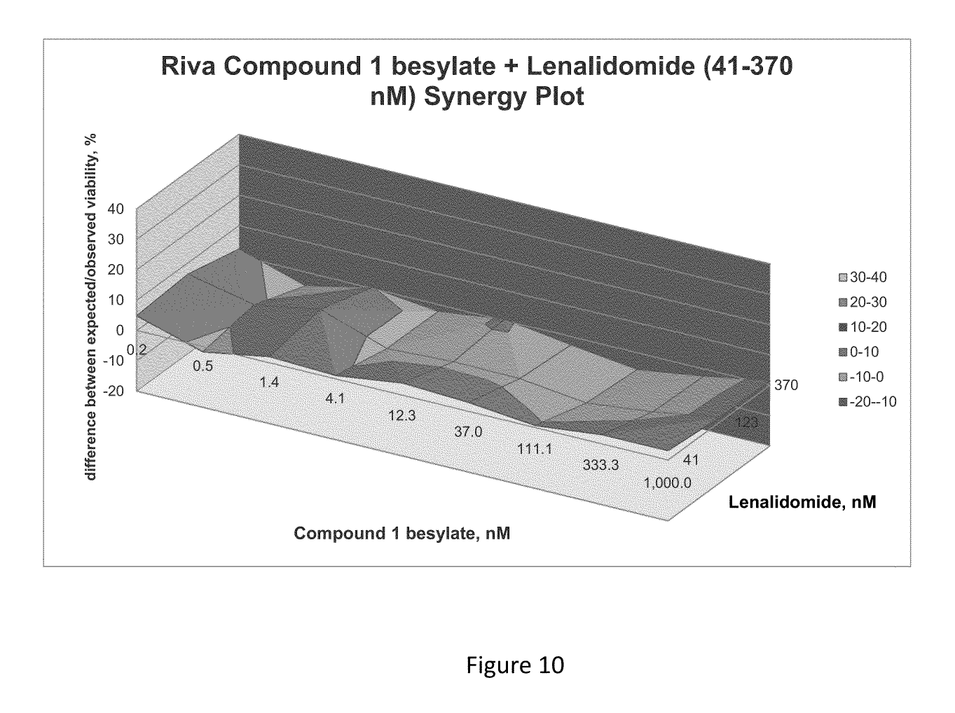

FIG. 9 presents another dose response curve for Compound 1 besylate and lenalidomide (333 nM) in the Riva cell line. Compound 1 besylate and lenalidomide in this Figure appear to be additive. FIG. 10 presents a “volcano” plot of another experiment of Compound 1 besylate and lenalidomide in the Riva cell line at lenalidomide concentrations of 41 nM, 123 nM and 370 nM. Compound 1 besylate and lenalidomide appear additive in FIG. 10. The data in FIGS. 7-10 appear to show that the combination of Compound 1 besylate and lenalidomide is not antagonistic, and may be synergistic.

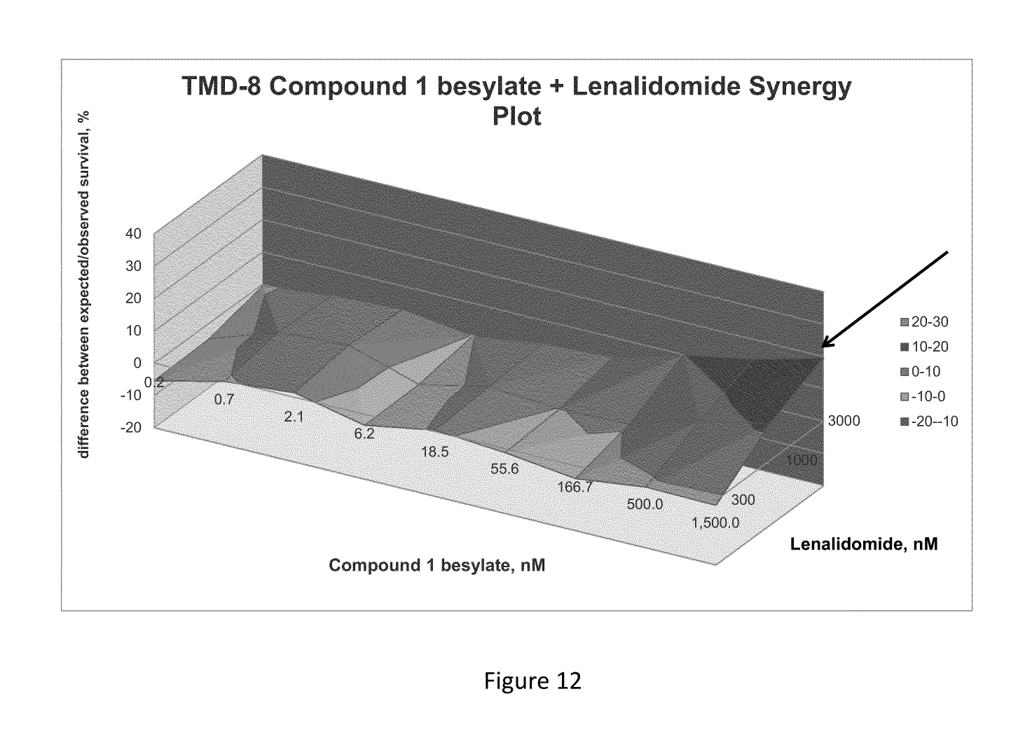

FIG. 11 presents a particular dose response curve for Compound 1 besylate and lenalidomide (3000 nM) in the TMD-8 cell line. Compound 1 besylate and lenalidomide appear synergistic (indicated by the arrow). FIG. 12 presents a “volcano” plot for a particular experiment with Compound 1 besylate and lenalidomide in the TMD-8 cell line at lenalidomide concentrations of 300 nM, 1000 nM and 3000 nM. Compound 1 besylate and lenalidomide in this Figure appear to be synergistic (indicated by the arrow). Lenalidomide was also tested in combination with Compound 1 besylate at concentrations of 100 nM, 300 nM and 1000 nM, and apparent synergy was confirmed (data not shown).

Compound 1 besylate and lenalidomide appear additive in the OCI-LY-10 (FIG. 1 and FIG. 2) and WSU-DLCL2 (FIG. 3 and FIG. 4) cell lines.

Compound 1 besylate and lenalidomide appear synergistic in the Riva cell line at a concentration of 3333 nM of lenalidomide in FIG. 5 and FIG. 6. Compound 1 besylate and lenalidomide in FIGS. 5 and 6 appear to be synergistic (indicated by the arrow). Compound 1 besylate and lenalidomide show some apparent synergy at a concentration of 333 nM of lenalidomide in FIGS. 7 and 8. Compound 1 besylate and lenalidomide appear to be additive at a concentration of 333 nM of lenalidomide in FIGS. 9 and 10. Compound 1 besylate and lenalidomide are confirmed to be synergistic in the TMD-8 cell line in FIG. 11 and FIG. 12. Accordingly, Compound 1 besylate and lenalidomide appear to show synergistic effects in at least the TMD-8 cell line. Some evidence of synergy appears also to be observed in the Riva cell line.

### Example 3

One particular irreversible BTK inhibitor, Compound 2, was screened against 342 kinases to ascertain kinase activity and/or selectivity:

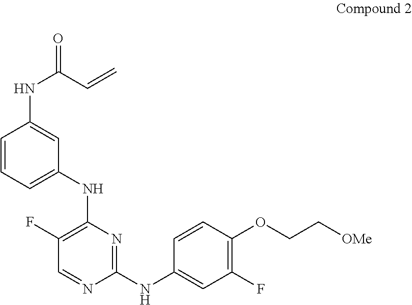 [SMILES: C=CC(=O)Nc1cccc(Nc2nc(Nc3ccc(OCCOC)c(F)c3)ncc2F)c1]

The binding assay system for profiling kinase activity were based upon HotSpot technology (Reaction Biology Corp.; Malvern, Pa., USA) and utilized radio-isotope-based P81 filtration. Compound 2 was dissolved in pure DMSO to make a 10 mM stock solution and serial dilutions were performed to a final 3 μM test concentration. Substrates for the various kinases tested against Compound 2 (substrate information available on the Reaction Biology Corp. website) were prepared fresh daily in Reaction Buffer. Any required cofactors were then added to the substrate solution. The identification and selection of the appropriate cofactor for each kinase is within the ability of a person skilled in the art. See, for example, Handbook of Assay Development in Drug Discovery, Ed. Lisa K. Minor, 2006: CRC Press, Boca Raton, Fla.; Gao et al., “A broad activity screen in support of a chemogenomic map for kinase signalling research and drug discovery,” Biochem J. 2013, 451(2): 313-28; and Eglen et al., “Drug discovery and the human kinome: Recent trends,” Pharmacology & Therapeutics 2011, 130(2): 144-156. The kinase was then added to the substrate solution and gently mixed. Compound 2 (5 nL) was then added to the kinase reaction mixture by acoustical droplet ejection and preincubated for 30 min at room temperature. ³³P-ATP (100 μM) was delivered into the reaction mixture to initiate the reaction. This was followed by incubation at room temperature for 2 h. The reaction was terminated and any unreacted phosphate was washed away using 0.1% phosphoric acid prior to detection utilizing a proprietary technology. The study was performed in duplicate and staurosporine, a non-selective, ATP-competitive kinase inhibitor, was used a the positive control in a 10-dose 1050 mode with 3-fold serial dilutions starting at 1 μM, 50 μM or 100 μM. DMSO was used as the negative control.

Determination of Percent Inhibition.

Percent inhibition of a kinase by a test compound, e.g., Compound 2, was determined according to the following formula: percent inhibition=[(kinase activity of negative control)−(kinase activity in presence of a test compound, e.g., Compound 2)/(kinase activity of negative control)]×100. Percent inhibition was expressed as an average where the assay was performed more than once, e.g., in duplicate.

Table 5 sets forth the average percent inhibition for Compound 2 against various kinases:

TABLE 5
Average Percent Inhibition of Kinases by Compound 2

| | % Enzyme Activity | | |
|---|---|---|---|
| | (relative to DMSO | | |
| | controls) | | Average % |
| Kinase | Run 1 | Run 2 | Inhibition |
| ABL1 | 45.37 | 45.23 | 54.70 |
| ABL2/ARG | 37.26 | 37.07 | 62.84 |
| ACK1 | 22.59 | 22.50 | 77.46 |
| AKT1 | 95.13 | 93.13 | 5.87 |
| AKT2 | 93.65 | 97.89 | 4.23 |
| AKT3 | 110.81 | 102.89 | −6.85 |
| ALK | 82.52 | 83.61 | 16.94 |
| ALK1/ACVRL1 | 124.78 | 123.91 | −24.35 |
| ALK2/ACVR1 | 385.44 | 385.47 | −285.46 |
| ALK3/BMPR1A | 104.30 | 104.51 | −4.41 |
| ALK4/ACVR1B | 140.52 | 143.80 | −42.16 |
| ALK5/TGFBR1 | 106.06 | 110.00 | −8.03 |
| ALK6/BMPR1B | 176.94 | 183.87 | −80.41 |
| ARAF | 102.92 | 100.02 | −1.47 |
| ARK5/NUAK1 | 13.26 | 13.31 | 86.72 |
| ASK1/MAP3K5 | 100.91 | 102.32 | −1.62 |
| Aurora A | 12.26 | 11.03 | 88.35 |
| Aurora B | 13.18 | 12.94 | 86.94 |
| Aurora C | 26.24 | 29.79 | 71.98 |
| AXL | 48.58 | 48.79 | 51.31 |
| BLK | 6.30 | 6.37 | 93.66 |
| BMPR2 | 83.77 | 83.85 | 16.19 |
| BMX/ETK | 0.93 | 1.23 | 98.92 |
| BRAF | 100.83 | 99.10 | 0.04 |
| BRK | 25.10 | 24.97 | 74.96 |
| BRSK1 | 87.10 | 89.40 | 11.75 |
| BRSK2 | 90.99 | 91.69 | 8.66 |
| BTK | 3.69 | 5.40 | 95.45 |
| CAMK1a | 109.10 | 111.65 | −10.38 |
| CAMK1b | 103.91 | 104.95 | −4.43 |
| CAMK1d | 103.60 | 103.13 | −3.36 |
| CAMK1g | 99.95 | 101.49 | −0.72 |
| CAMK2a | 109.14 | 110.06 | −9.60 |
| CAMK2b | 96.40 | 95.81 | 3.89 |
| CAMK2d | 123.75 | 122.81 | −23.28 |
| CAMK2g | 103.51 | 110.85 | −7.18 |
| CAMK4 | 102.16 | 102.35 | −2.26 |
| CAMKK1 | 93.48 | 87.89 | 9.31 |
| CAMKK2 | 74.65 | 71.82 | 26.77 |
| CDC7/DBF4 | 100.26 | 103.78 | −2.02 |
| CDK1/cyclin A | 111.93 | 124.07 | −18.00 |
| CDK1/cyclin B | 87.16 | 87.03 | 12.91 |
| CDK1/cyclin E | 100.38 | 102.23 | −1.30 |
| CDK16/cyclin Y | 104.10 | 103.36 | −3.73 |
| CDK2/cyclin A | 85.83 | 86.86 | 13.66 |
| CDK2/cyclin A1 | 78.25 | 77.74 | 22.00 |
| CDK2/cyclin E | 87.92 | 92.03 | 10.02 |
| CDK3/cyclin E | 78.15 | 76.95 | 22.45 |
| CDK4/cyclin D1 | 95.58 | 96.28 | 4.07 |
| CDK4/cyclin D3 | 95.54 | 94.15 | 5.15 |
| CDK5/p25 | 89.55 | 92.26 | 9.10 |
| CDK5/p35 | 94.97 | 96.06 | 4.49 |
| CDK6/cyclin D1 | 101.41 | 100.42 | −0.92 |
| CDK6/cyclin D3 | 99.55 | 100.13 | 0.16 |
| CDK7/cyclin H | 98.10 | 97.18 | 2.36 |
| CDK9/cyclin K | 81.70 | 82.16 | 18.07 |
| CDK9/cyclin T1 | 87.76 | 91.54 | 10.35 |
| CHK1 | 93.14 | 94.56 | 6.15 |
| CHK2 | 25.85 | 25.28 | 74.43 |
| CK1a1 | 107.76 | 105.57 | −6.67 |
| CK1d | 99.87 | 100.20 | −0.03 |
| CK1epsilon | 101.51 | 102.41 | −1.96 |
| CK1g1 | 88.77 | 90.27 | 10.48 |
| CK1g2 | 89.13 | 85.74 | 12.57 |
| CK1g3 | 84.15 | 85.84 | 15.01 |
| CK2a | 117.05 | 123.24 | −20.15 |
| CK2a2 | 98.25 | 105.38 | −1.81 |
| c−Kit | 77.47 | 79.45 | 21.54 |
| CLK1 | 74.39 | 77.44 | 24.08 |
| CLK2 | 50.21 | 50.65 | 49.57 |
| CLK3 | 90.36 | 95.83 | 6.90 |
| CLK4 | 57.40 | 53.92 | 44.34 |
| c−MER | 66.48 | 66.40 | 33.56 |
| c−MET | 102.62 | 100.36 | −1.49 |
| COT1/MAP3K8 | 101.72 | 100.94 | −1.33 |
| CSK | 81.71 | 81.81 | 18.24 |
| c−Src | 28.17 | 27.88 | 71.97 |
| CTK/MATK | 102.20 | 103.87 | −3.03 |
| DAPK1 | 102.79 | 93.48 | 1.86 |
| DAPK2 | 108.05 | 111.72 | −9.89 |
| DCAMKL1 | 98.43 | 97.52 | 2.02 |
| DCAMKL2 | 100.15 | 99.50 | 0.18 |
| DDR1 | 25.70 | 24.12 | 75.09 |
| DDR2 | 102.90 | 104.85 | −3.87 |
| DLK/MAP3K12 | 74.17 | 80.18 | 22.82 |
| DMPK | 104.46 | 102.05 | −3.25 |
| DMPK2 | 97.36 | 99.56 | 1.54 |
| DRAK1/STK17A | 82.93 | 80.57 | 18.25 |
| DYRK1/DYRK1A | 87.66 | 88.41 | 11.96 |
| DYRK1B | 78.60 | 80.92 | 20.24 |
| DYRK2 | 60.98 | 62.12 | 38.45 |
| DYRK3 | 85.99 | 85.89 | 14.06 |
| DYRK4 | 105.18 | 105.53 | −5.35 |
| EGFR | 19.23 | 20.17 | 80.30 |
| EPHA1 | 99.47 | 101.01 | −0.24 |
| EPHA2 | 84.23 | 84.12 | 15.82 |
| EPHA3 | 96.21 | 100.18 | 1.81 |
| EPHA4 | 92.86 | 88.96 | 9.09 |
| EPHA5 | 89.11 | 93.57 | 8.66 |
| EPHA6 | 95.52 | 102.01 | 1.24 |
| EPHA7 | 60.01 | 64.14 | 37.93 |
| EPHA8 | 94.25 | 93.79 | 5.98 |
| EPHB1 | 79.45 | 79.66 | 20.44 |
| EPHB2 | 104.27 | 106.64 | −5.45 |
| EPHB3 | 99.17 | 98.21 | 1.31 |
| EPHB4 | 81.25 | 81.45 | 18.65 |
| ERBB2/HER2 | 39.81 | 37.50 | 61.34 |
| ERBB4/HER4 | 9.15 | 8.32 | 91.27 |
| ERK1 | 97.44 | 99.93 | 1.32 |
| ERK2/MAPK1 | 105.72 | 102.79 | −4.25 |
| ERK5/MAPK7 | 100.83 | 99.91 | −0.37 |
| ERK7/MAPK15 | 63.77 | 66.05 | 35.09 |
| FAK/PTK2 | 62.84 | 62.86 | 37.15 |
| FER | 88.07 | 88.26 | 11.83 |
| FES/FPS | 63.95 | 65.95 | 35.05 |
| FGFR1 | 41.38 | 39.31 | 59.66 |
| FGFR2 | 37.28 | 35.97 | 63.37 |
| FGFR3 | 32.14 | 31.78 | 68.04 |
| FGFR4 | 66.32 | 63.44 | 35.12 |
| FGR | 39.73 | 40.47 | 59.90 |
| FLT1/VEGFR1 | 83.62 | 79.20 | 18.59 |
| FLT3 | 3.19 | 3.25 | 96.78 |
| FLT4/VEGFR3 | 43.82 | 44.24 | 55.97 |
| FMS | 64.50 | 67.34 | 34.08 |
| FRK/PTK5 | 100.54 | 97.96 | 0.75 |
| FYN | 81.08 | 81.84 | 18.54 |
| GCK/MAP4K2 | 100.27 | 100.02 | −0.14 |
| GLK/MAP4K3 | 102.03 | 108.13 | −5.08 |
| GRK1 | 103.55 | 103.40 | −3.47 |
| GRK2 | 104.15 | 103.91 | −4.03 |
| GRK3 | 99.45 | 100.95 | −0.20 |
| GRK4 | 107.15 | 106.07 | −6.61 |
| GRK5 | 103.03 | 102.15 | −2.59 |
| GRK6 | 102.34 | 103.73 | −3.03 |
| GRK7 | 89.14 | 90.93 | 9.96 |
| GSK3a | 75.97 | 74.98 | 24.53 |
| GSK3b | 121.82 | 122.56 | −22.19 |
| Haspin | 90.56 | 89.91 | 9.77 |
| HCK | 71.99 | 68.97 | 29.52 |
| HGK/MAP4K4 | 98.87 | 98.60 | 1.27 |
| HIPK1 | 87.63 | 91.73 | 10.32 |
| HIPK2 | 95.75 | 98.61 | 2.82 |
| HIPK3 | 112.11 | 118.15 | −15.13 |
| HIPK4 | 93.61 | 93.78 | 6.31 |
| HPK1/MAP4K1 | 85.77 | 88.42 | 12.90 |
| IGF1R | 82.83 | 85.99 | 15.59 |
| IKKa/CHUK | 91.43 | 91.50 | 8.54 |
| IKKb/IKBKB | 95.44 | 97.06 | 3.75 |
| IKKe/IKBKE | 78.26 | 78.18 | 21.78 |
| IR | 84.52 | 84.68 | 15.40 |
| IRAK1 | 76.49 | 74.41 | 24.55 |
| IRAK4 | 88.61 | 86.52 | 12.43 |
| IRR/INSRR | 88.90 | 90.97 | 10.07 |
| ITK | 7.97 | 7.95 | 92.04 |
| JAK1 | 59.70 | 59.03 | 40.63 |
| JAK2 | 108.64 | 114.63 | −11.63 |
| JAK3 | 2.53 | 2.73 | 97.37 |
| JNK1 | 88.14 | 87.66 | 12.10 |
| JNK2 | 92.48 | 95.08 | 6.22 |
| JNK3 | 110.08 | 115.56 | −12.82 |
| KDR/VEGFR2 | 83.35 | 81.24 | 17.70 |
| KHS/MAP4K5 | 94.89 | 90.82 | 7.15 |
| LATS1 | 89.52 | 89.80 | 10.34 |
| LATS2 | 88.40 | 91.16 | 10.22 |
| LCK | 64.67 | 63.36 | 35.99 |
| LCK2/ICK | 100.94 | 95.10 | 1.98 |
| LIMK1 | 60.98 | 61.36 | 38.83 |
| LIMK2 | 101.00 | 100.73 | −0.86 |
| LKB1 | 99.24 | 95.94 | 2.41 |
| LOK/STK10 | 43.10 | 43.01 | 56.94 |
| LRRK2 | 33.02 | 35.12 | 65.93 |
| LYN | 71.67 | 73.24 | 27.54 |
| LYN B | 92.90 | 95.69 | 5.70 |
| MAPKAPK2 | 109.81 | 105.91 | −7.86 |
| MAPKAPK3 | 102.28 | 102.63 | −2.45 |
| MAPKAPK5/PRAK | 109.84 | 113.33 | −11.58 |
| MARK1 | 90.93 | 97.35 | 5.86 |
| MARK2/PAR-1Ba | 99.58 | 97.83 | 1.30 |
| MARK3 | 101.48 | 100.37 | −0.92 |
| MARK4 | 82.17 | 80.87 | 18.48 |
| MEK1 | 109.05 | 111.60 | −10.33 |
| MEK2 | 106.36 | 104.95 | −5.66 |
| MEK3 | 117.30 | 113.34 | −15.32 |
| MEKK1 | 112.92 | 116.55 | −14.74 |
| MEKK2 | 108.13 | 113.65 | −10.89 |
| MEKK3 | 101.68 | 106.39 | 4.03 |
| MELK | 108.80 | 107.34 | −8.07 |
| MINK/MINK1 | 100.72 | 97.49 | 0.90 |
| MKK4 | 116.84 | 116.35 | −16.60 |
| MKK6 | 96.36 | 97.41 | 3.11 |
| MLCK/MYLK | 95.57 | 95.24 | 4.59 |
| MLCK2/MYLK2 | 71.62 | 68.13 | 30.13 |
| MLK1/MAP3K9 | 14.50 | 14.05 | 85.72 |
| MLK2/MAP3K10 | 45.39 | 45.33 | 54.64 |
| MLK3/MAP3K11 | 27.89 | 25.23 | 73.44 |
| MNK1 | 97.78 | 94.26 | 3.98 |
| MNK2 | 83.04 | 83.21 | 16.88 |
| MRCKa/CDC42BPA | 113.51 | 115.15 | −14.33 |
| MRCKb/CDC42BPB | 106.78 | 105.47 | −6.12 |
| MSK1/RPS6KA5 | 94.87 | 99.72 | 2.70 |
| MSK2/RPS6KA4 | 103.35 | 97.38 | −0.36 |
| MSSK1/STK23 | 108.42 | 104.81 | −6.62 |
| MST1/STK4 | 70.20 | 68.65 | 30.58 |
| MST2/STK3 | 91.08 | 88.64 | 10.14 |
| MST3/STK24 | 86.56 | 84.46 | 14.49 |
| MST4 | 97.85 | 104.33 | −1.09 |
| MUSK | 70.16 | 68.05 | 30.89 |
| MYLK3 | 113.48 | 116.49 | −14.98 |
| MYO3b | 103.52 | 101.67 | −2.59 |
| NEK1 | 52.74 | 53.95 | 46.65 |
| NEK11 | 92.53 | 92.84 | 7.32 |
| NEK2 | 102.22 | 106.65 | −4.44 |
| NEK3 | 76.65 | 75.11 | 24.12 |
| NEK4 | 84.64 | 88.33 | 13.52 |
| NEK5 | 60.25 | 60.87 | 39.44 |
| NEK6 | 105.95 | 105.43 | −5.69 |
| NEK7 | 98.29 | 100.34 | 0.68 |
| NEK9 | 83.83 | 84.30 | 15.94 |
| NLK | 99.61 | 102.97 | −1.29 |
| OSR1/OXSR1 | 116.94 | 121.56 | −19.25 |
| P38a/MAPK14 | 105.80 | 107.56 | −6.68 |
| P38b/MAPK11 | 101.64 | 101.17 | −1.41 |
| P38d/MAPK13 | 98.94 | 99.78 | 0.64 |
| P38g | 105.35 | 105.71 | −5.53 |
| P70S6K/RPS6KB1 | 85.89 | 81.02 | 16.54 |
| P70S6Kb/RPS6KB2 | 95.60 | 96.10 | 4.15 |
| PAK1 | 96.27 | 98.58 | 2.58 |
| PAK2 | 95.53 | 94.87 | 4.80 |
| PAK3 | 91.86 | 95.22 | 6.46 |
| PAK4 | 98.70 | 96.40 | 2.45 |
| PAK5 | 122.54 | 132.33 | −27.43 |
| PAK6 | 79.30 | 84.76 | 17.97 |
| PASK | 94.65 | 93.08 | 6.14 |
| PBK/TOPK | 98.58 | 95.27 | 3.07 |
| PDGFRa | 63.93 | 62.78 | 36.65 |
| PDGFRb | 47.74 | 47.41 | 52.42 |
| PDK1/PDPK1 | 106.60 | 105.33 | −5.96 |
| PHKg1 | 85.80 | 85.18 | 14.51 |
| PHKg2 | 117.59 | 111.88 | −14.73 |
| PIM1 | 103.69 | 103.94 | −3.82 |
| PIM2 | 135.59 | 129.41 | −32.50 |
| PIM3 | 103.69 | 99.97 | −1.83 |
| PKA | 85.81 | 85.13 | 14.53 |
| PKAcb | 49.70 | 51.07 | 49.62 |
| PKAcg | 127.92 | 127.73 | −27.82 |
| PKCa | 88.40 | 88.80 | 11.40 |
| PKCb1 | 72.71 | 72.06 | 27.62 |
| PKCb2 | 50.13 | 48.85 | 50.51 |
| PKCd | 100.41 | 96.02 | 1.78 |
| PKCepsilon | 93.22 | 94.21 | 6.29 |
| PKCeta | 108.82 | 116.79 | −12.81 |
| PKCg | 83.88 | 84.16 | 15.98 |
| PKCiota | 105.24 | 105.70 | −5.47 |
| PKCmu/PRKD1 | 74.85 | 75.85 | 24.65 |
| PKCnu/PRKD3 | 80.06 | 79.79 | 20.07 |
| PKCtheta | 83.65 | 84.12 | 16.12 |
| PKCzeta | 99.81 | 95.15 | 2.52 |
| PKD2/PRKD 2 | 86.37 | 86.22 | 13.70 |
| PKG1a | 87.99 | 94.55 | 8.73 |
| PKG1b | 85.36 | 87.08 | 13.78 |
| PKG2/PRKG2 | 87.22 | 84.45 | 14.16 |
| PKN1/PRK1 | 93.83 | 92.30 | 6.94 |
| PKN2/PRK2 | 93.30 | 91.42 | 7.64 |
| PKN3/PRK3 | 106.24 | 108.40 | −7.32 |
| PLK1 | 91.11 | 92.77 | 8.06 |
| PLK2 | 86.63 | 86.74 | 13.32 |
| PLK3 | 96.95 | 100.49 | 1.28 |
| PLK4/SAK | 54.75 | 55.16 | 45.04 |
| PRKX | 97.87 | 98.53 | 1.80 |
| PYK2 | 70.59 | 70.13 | 29.64 |
| RAF1 | 83.34 | 83.62 | 16.52 |
| RET | 13.17 | 13.63 | 86.60 |
| RIPK2 | 77.59 | 75.01 | 23.70 |
| RIPK3 | 116.80 | 120.69 | −18.75 |
| RIPK5 | 96.13 | 99.22 | 2.32 |
| ROCK1 | 107.64 | 105.14 | −6.39 |
| ROCK2 | 102.29 | 101.47 | −1.88 |
| RON/MST1R | 103.11 | 101.37 | −2.24 |
| ROS/ROS1 | 13.52 | 13.26 | 86.61 |
| RSK1 | 73.29 | 72.45 | 27.13 |
| RSK2 | 82.57 | 84.26 | 16.59 |
| RSK3 | 85.80 | 85.16 | 14.52 |
| RSK4 | 77.21 | 77.21 | 22.79 |
| SGK1 | 99.71 | 99.55 | 0.37 |
| SGK2 | 71.38 | 76.59 | 26.02 |
| SGK3/SGKL | 99.54 | 105.31 | −2.42 |
| SIK1 | 48.40 | 48.72 | 51.44 |
| SIK2 | 56.26 | 57.07 | 43.34 |
| SIK3 | 91.33 | 92.80 | 7.93 |
| SLK/STK2 | 74.89 | 75.27 | 24.92 |
| SNARK/NUAK2 | 83.70 | 84.92 | 15.69 |
| SRMS | 123.69 | 122.52 | −23.11 |
| SRPK1 | 98.81 | 96.73 | 2.23 |
| SRPK2 | 90.92 | 89.61 | 9.73 |
| SSTK/TSSK6 | 107.37 | 99.84 | −3.60 |
| STK16 | 22.93 | 21.04 | 78.01 |
| STK22D/TSSK1 | 88.04 | 89.72 | 11.12 |
| STK25/YSK1 | 94.47 | 94.10 | 5.72 |
| STK32B/YANK2 | 95.81 | 93.60 | 5.30 |
| STK32C/YANK3 | 104.94 | 107.08 | −6.01 |
| STK33 | 51.19 | 52.54 | 48.14 |
| STK38/NDR1 | 92.71 | 93.70 | 6.79 |
| STK38L/NDR2 | 106.68 | 95.97 | −1.33 |
| STK39/STLK3 | 91.63 | 92.89 | 7.74 |
| SYK | 78.04 | 77.10 | 22.43 |
| TAK1 | 73.39 | 71.98 | 27.32 |
| TAOK1 | 100.50 | 96.25 | 1.63 |
| TAOK2/TAO1 | 98.49 | 94.44 | 3.53 |
| TAOK3/JIK | 94.52 | 90.00 | 7.74 |
| TBK1 | 58.13 | 59.19 | 41.34 |
| TEC | 10.36 | 11.47 | 89.08 |
| TESK1 | 97.22 | 98.52 | 2.13 |
| TGFBR2 | 98.07 | 102.31 | −0.19 |
| TIE2/TEK | 102.88 | 107.55 | −5.21 |
| TLK1 | 102.43 | 103.40 | −2.91 |
| TLK2 | 107.74 | 104.89 | −6.32 |
| TNIK | 66.84 | 67.77 | 32.69 |
| TNK1 | 15.91 | 16.61 | 83.74 |
| TRKA | 117.40 | 117.94 | −17.67 |
| TRKB | 86.53 | 85.00 | 14.24 |
| TRKC | 35.08 | 32.34 | 66.29 |
| TSSK2 | 97.01 | 96.27 | 3.36 |
| TSSK3/STK22C | 132.55 | 132.59 | −32.57 |
| TTBK1 | 101.11 | 102.19 | −1.65 |
| TTBK2 | 102.05 | 99.67 | −0.86 |
| TXK | 0.42 | 0.03 | 99.77 |
| TYK1/LTK | 77.75 | 75.81 | 23.22 |
| TYK2 | 48.20 | 46.63 | 52.58 |
| TYRO3/SKY | 95.11 | 96.84 | 4.03 |
| ULK1 | 100.12 | 101.06 | −0.59 |
| ULK2 | 102.27 | 109.21 | −5.74 |
| ULK3 | 79.58 | 76.98 | 21.72 |
| VRK1 | 84.28 | 89.86 | 12.93 |
| VRK2 | 94.00 | 95.88 | 5.06 |
| WEE1 | 73.06 | 74.89 | 26.03 |
| WNK1 | 112.15 | 112.04 | −12.10 |
| WNK2 | 92.35 | 95.28 | 6.19 |
| WNK3 | 91.35 | 91.97 | 8.34 |
| YES/YES1 | 18.72 | 18.27 | 81.50 |
| ZAK/MLTK | 83.71 | 85.00 | 15.65 |
| ZAP70 | 110.35 | 108.07 | −9.21 |
| ZIPK/DAPK3 | 109.39 | 113.13 | −11.26 |

### Example 4

### Dose Escalation Study with Rituximab

Capsules comprising N-(3-(5-fluoro-2-(4-(2-methoxyethoxyl)phenylamino)pyrimidin-4-ylamino)phenyl)acrylamide besylate correspond to Table 1 in Example 1.

Lenalidomide capsules correspond to those listed in Tables 2 and 3 in Example 1.

Rituximab is provided to the physician/investigator in 10 mg/mL vials comprising 100 mg/10 mL or 500 mg/50 mL. Prior to administration, rituximab is diluted to a dose of 1 mg/mL, 2 mg/mL, 3 mg/mL or 4 mg/mL with either 5% dextrose in water or 0.9% sodium chloride. Rituximab is thereafter administered as a 1 mg/mL to 4 mg/mL infusion according to the dosages set forth in Table 6, below.

Study Design

Subjects with relapsed or refractory B-cell non-Hodgkin lymphoma, excluding SLL, CLL and Waldenstrom Macroglobulinemia, who have failed at least one prior treatment regimen will be enrolled in a “3+3” dose escalation and expansion study to determine the Not Tolerated Dose (NTD), the Optimal Biologic Effect dose (OBE) and the Maximum Tolerated Dose (MTD) of Compound 1, lenalidomide and rituximab. Approximately 30-42 patients are expected to be enrolled in the study.

Study treatment will be administered in 28-day cycles at specified dose levels as scheduled until disease progression, unacceptable toxicity, or discontinuation for any other reason. Subjects will continue on the starting dose until the preliminary recommended Phase 2 dose (RP2D) is determined, at which point they can be switched to the preliminary RP2D.

N-(3-(5-fluoro-2-(4-(2-methoxyethoxyl)phenylamino)pyrimidin-4-ylamino)phenyl)acrylamide besylate (Compound 1 besylate), lenalidomide and rituximab will be administered according to the cohort listed in Table 6:

TABLE 6
Study Dosing Schema for Study

| | Compound 1 | | |
|---|---|---|---|
| COHORT | besylate | Lenalidomide | Rituximab |
| 1 | 375 mg BID or | 15 mg QD or 20 | 375 mg/m² on day 1 of |
| | 500 mg BID on | mg QD on days | each cycle |
| | days 1-28 | 1-21 | |

Subjects will be treated PO (oral) BID (twice daily) with Compound 1 besylate and lenalidomide during an initial 28-day treatment cycle. Rituximab will be administered as a single intravenous (IV) infusion of 375 mg/m² on day 1 of each 28-day cycle. Subjects will continue on treatment until disease progression, unacceptable toxicity, or discontinuation for any other reason and will be assessed for safety, tolerability and DLT as well as pharmacokinetic (“PK”), pharmacodynamic (“PD”), and disease response.

We claim:

1. A method of treating, stabilizing or lessening the severity or progression of a B-cell non-Hodgkin lymphoma comprising administering to a patient in need thereof an irreversible BTK inhibitor and lenalidomide, wherein the irreversible BTK inhibitor has not more than about 50% inhibition of a kinase selected from c-Kit, PDGFRa, RIPK2, HCK, EPHA6, LYN, CSK, LCK, ZAK/MLTK, LYN B, FRK/PTK5, FYN, BRAF, RIPK3, ARAF and SRMS, or combinations thereof.

2. The method according to claim 1, wherein the irreversible BTK inhibitor has not more than about 30% inhibition of a kinase selected from c-Kit, RIPK2, HCK, EPHA6, LYN, CSK, ZAK/MLTK, LYN B, FRK/PTK5, FYN, BRAF, RIPK3, ARAF and SRMS, or combinations thereof.

3. The method according to claim 1, wherein the irreversible BTK inhibitor has not more than about 10% inhibition of a kinase selected from EPHA6, LYN B, FRK/PTK5, BRAF, RIPK3, ARAF and SRMS, or combinations thereof.

4. The method according to claim 1, wherein the irreversible BTK inhibitor has a percent inhibition of LYN that is not more than about 20-30%.

5. A method of treating, stabilizing or lessening the severity or progression of a B-cell non-Hodgkin lymphoma comprising administering to a patient in need thereof Compound 1 (N-(3-(5-fluoro-2-(4-(2-methoxyethoxyl)phenylamino)pyrimidin-4-ylamino)phenyl)acrylamide): 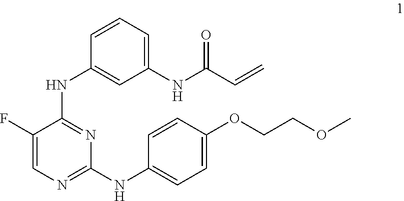 [SMILES: C=CC(=O)Nc1cccc(Nc2nc(Nc3ccc(OCCOC)cc3)ncc2F)c1]
or a pharmaceutically acceptable salt thereof, lenalidomide and an anti-CD20 antibody.

6. The method according to claim 5, wherein the B-cell non-Hodgkin lymphoma is selected from follicular lymphoma and marginal zone lymphoma.

7. The method according to claim 6, wherein marginal zone lymphoma is selected from nodal marginal zone lymphoma, extranodal marginal zone lymphoma of mucosa-associated lymphoid tissue and splenic marginal zone lymphoma.

8. The method according to claim 5, wherein the B-cell non-Hodgkin lymphoma is selected from diffuse large B-cell non-Hodgkin lymphoma and mantle cell lymphoma.

9. The method according to claim 5, wherein Compound 1 is administered twice a day.

10. The method according to claim 7, wherein Compound 1 is administered on each day of a 28-day cycle.

11. The method according to claim 10, wherein lenalidomide is administered once a day.

12. The method according to claim 5, wherein the anti-CD20 antibody is rituximab.

13. The method according to claim 12, wherein rituximab is administered once during a 28-day cycle.

14. The method according to claim 13, wherein rituximab is administered as an intravenous infusion.

15. The method according to claim 9, wherein Compound 1 is in the form of a benzenesulfonic acid salt.

16. The method of claim 15, wherein each of Compound 1 and lenalidomide is administered as an oral dosage form.

17. A method of preventing, treating, stabilizing or lessening the severity or progression of a B-cell non-Hodgkin lymphoma, the method comprising administering to a patient in need thereof therapeutically effective amounts of each of Compound 1, or a pharmaceutically acceptable salt thereof, lenalidomide and an anti-CD20 antibody, wherein the therapeutically effective amount of Compound 1 is about 750 mg to about 1000 mg per day.

18. The method according to claim 17, wherein the therapeutically effective amount of Compound 1 is about 375 mg BID.

19. The method according to claim 17, wherein the therapeutically effective amount Compound 1 is about 500 mg BID.

20. The method according to claim 17, wherein lenalidomide is administered once a day (QD).

21. The method according to claim 20, wherein the therapeutically effective amount of lenalidomide is selected from about 15 mg, about 20 mg or about 25 mg.

22. The method according to claim 17, wherein the anti-CD20 antibody is rituximab

23. A system for treating, stabilizing or lessening the severity of a B-cell non-Hodgkin lymphoma, the system comprising Compound 1, or a pharmaceutically acceptable salt thereof, lenalidomide and an anti-CD20 antibody.

24. The method according to claim 23, wherein the anti-CD20 antibody is rituximab.
